(19) United States
(12) Patent Application Publication
(10) Pub. No.: US 2025/0014750 A1
(43) Pub. Date: Jan. 9, 2025

(54) APPARATUS AND METHODS FOR A KNOWLEDGE PROCESSING SYSTEM THAT APPLIES A REASONING TECHNIQUE FOR CELL-BASED ANALYSIS TO PREDICT A CLINICAL OUTCOME

(71) Applicant: Notch Therapeutics (Canada) Inc., Vancouver (CA)

(72) Inventors: Daniel Christopher KIROUAC, Alameda, CA (US); Cole M. J. ZMURCHOK, Vancouver (CA); Jordan T. SICHERMAN, Vancouver (CA); Avisek DEYATI, Vancouver (CA)

(73) Assignee: Notch Therapeutics (Canada) Inc., Vancouver, BC (CA)

(21) Appl. No.: 18/884,204

(22) Filed: Sep. 13, 2024

Related U.S. Application Data
(63) Continuation of application No. PCT/US2023/064338, filed on Mar. 14, 2023.
(60) Provisional application No. 63/405,128, filed on Sep. 9, 2022.
(60) Provisional application No. 63/319,547, filed on Mar. 14, 2022.

(51) Int. Cl. G16H 50/20; G16B 25/10; G16B 40/20; G16H 20/10

(52) U.S. Cl. CPC G16H 50/20; G16B 25/10; G16B 40/20; G16H 20/10

(57) ABSTRACT

In some implementations, a machine learning model (e.g., a knowledge processing system) can apply a reasoning technique to knowledge representations associated with cells (e.g., using a genetic algorithm) and predict a clinical outcome (e.g., for a patient). In some implementations, a method includes producing a cell population. Gene expression data for the cell population can be received. The gene expression data can be analyzed to identify a set of gene signatures. The set of gene signatures can be provided as an input to a machine learning classifier. A predicted clinical outcome can be generated.

### CROSS-REFERENCE TO RELATED APPLICATIONS

This application is a Continuation of International Patent Application No. PCT/US2023/064338, filed Mar. 14, 2023, which claims priority to and the benefit of U.S. Provisional Application No. 63/319,547, filed Mar. 14, 2022 and titled “APPARATUS AND METHODS FOR A KNOWLEDGE PROCESSING SYSTEM THAT APPLIES A REASONING TECHNIQUE FOR CELL-BASED ANALYSIS TO PREDICT A CLINICAL OUTCOME” and U.S. Provisional Application No. 63/405,128, filed Sep. 9, 2022 and titled “APPARATUS AND METHODS FOR A KNOWLEDGE PROCESSING SYSTEM THAT APPLIES A REASONING TECHNIQUE FOR CELL-BASED ANALYSIS TO PREDICT A CLINICAL OUTCOME.” each of which is incorporated herein by reference in its entirety.

### FIELD

The present disclosure relates to an apparatus(es) and method(s) for a machine learning model (e.g., a knowledge processing system) that can apply a reasoning technique for cell-based analysis to predict a clinical outcome (e.g., for a patient).

### BACKGROUND OF THE DISCLOSURE

Chimeric Antigen Receptor T-cells (CAR-T) have shown appreciable activity in the treatment of B cell malignancies. However, T cells can bring unique challenges to therapeutic development. These so-called “living drugs” can proliferate, differentiate. actively traffic between tissues, and/or engage in two-way communication with a patient immune system while executing function. The resultant pharmacology can be different from that of small molecules or biologics in terms of, for example, the relationship between administered dose and exposure.

The pharmacokinetics (‘cellular kinetics’) of circulating CAR-Ts can be characterized by three distinct phases: initial expansion, followed by a rapid contraction then slow, long-term decay. The degree of cell expansion (Cmax) and long-term exposure (AUC) can vary between patients (e.g., ˜3 orders of magnitude) and can be predictive of efficacy (tumor size reduction) and/or toxicity. However, the product-and host-intrinsic factors mediating this pharmacology remain non-optimally defined. As such, facts and/or relationships (i.e., knowledge representations) associated with the CAR-Ts may not be optimally known, understood, and/or leveraged (e.g., by a knowledge processing system). Some known empirical, non-linear mixed effects models to quantify the pharmacokinetics of Kymriah® (tisagenlecleul, CTL019) have been provided as part of the Biologics License Application (BLA). Such a known formulation has been applied to other CAR-T therapies in a variety of indications, and has been adopted by the FDA for benchmarking. While potentially applicable for quantifying clinical data. the empirical equations do not account for the underlying biology. and thus are of lesser value in simulating the effects of alternate CAR-T designs or treatment regimens. In that context, a mathematical model capable of quantitatively describing clinical data. while based on sound biological mechanisms would be desirable in the development of novel CAR-T products, analogous to the use of mechanism-based pharmacokinetic-pharmacodynamic (PKPD) modelling (quantitative systems pharmacology) during the exploratory phases of drug development.

Some known models are derived from predator-prey equations. wherein the CAR-T cells are endowed with unlimited proliferation capacity. Such known models, however, neither address the clinical observation that CAR-T expansion capacity varies drastically between patients. nor the mechanisms underlying such clinical variability.

It is desirable to obviate or mitigate one or more of the above deficiencies.

### SUMMARY OF THE DISCLOSURE

In some implementations, a machine learning model (e.g., a knowledge processing system) can apply a reasoning technique to knowledge representations associated with cells (e.g., using a genetic algorithm) and predict a clinical outcome (e.g., for a patient). In some implementations, a method comprises receiving gene expression data for a plurality of cells. Differential gene expression analysis can be conducted based on the gene expression data to generate differential gene expression data. Per-sample gene signature enrichment for a plurality of identified biological pathways are estimated based on the differential gene expression data. Gene signatures having a differential enrichment pattern can be filtered based on a pre-determined threshold between groups of responders and non-responders to a treatment. Filtered gene signatures can be grouped into a plurality of groups based on pairwise correlations between gene signature enrichment scores. Iteratively performing a predefined number of times to define a plurality of sets of gene signatures, a gene signature from each group from the plurality of groups can be randomly selected to define a set of gene signatures from the plurality of sets of gene signatures. The plurality of sets of gene signatures can be provided as an input to a feature selection algorithm to define a feature vector including a reduced set of gene signatures identified from the plurality of sets of gene signatures. A machine learning classifier can be trained using the feature vector.

### BRIEF DESCRIPTION OF THE DRAWINGS

FIG. 1 shows an antigen toggle-switch model of T cell regulation that quantitatively describes PKPD behavior of complete responder (CR), partial responder (PR), and non-responder (NR) patient population response to Kymriah® in Chronic Lymphocytic leukemia (CLL), according to an embodiment.

FIG. 2 shows single-sample Gene Set Enrichment Analysis (ssGSEA) estimates of the activity of signaling pathways and enrichment of cell populations in CAR-Ts, separated by response, according to an embodiment.

FIG. 3 shows single cell RNA sequencing of twelve pre-infusion CAR-T products classified by response, according to an embodiment.

FIG. 4 shows Kymriah (R) response predictions using a ssGSEA-based classifier, according to an embodiment.

FIG. 5 shows clinical variability in dose, tumor burden, and CR/PR/NR pharmacological archetype for population variance in exposure to Kymriah®, and predict clinical covariates of response to Yescarta®), according to an embodiment.

FIG. 6 shows model training, analysis, and test associated with Abecma® dose response, according to an embodiment.

FIG. 7A shows a flowchart of a response classifier workflow, according to an embodiment.

FIG. 7B shows a pictorial representation of the response classifier workflow, showing example data at each step in the pipeline using a process similar to that discussed with respect to FIG. 7A, according to an embodiment.

FIG. 7C shows a flowchart of a response classifier workflow, according to an embodiment.

FIG. 7D shows a pictorial representation of the response classifier workflow, showing example data at each step in the pipeline using a process similar to that discussed with respect to FIG. 7C, according to an embodiment.

FIG. 8 shows a PKPD model workflow, according to an embodiment.

FIG. 9 shows goodness-of-fit plots for Kymriah® model fitting in FIG. 1, according to an embodiment.

FIG. 10 shows Local Parameter Sensitivity Analysis of CR/PR/NR populations, according to an embodiment.

FIG. 11 shows volcano plot of differentially expressed genes between CR vs. NR groups, according to an embodiment.

FIG. 12 shows indications of select gene sets differentially enriched between CR vs. NR groups, according to an embodiment.

FIG. 13 shows (A) Mean receiver operating characteristic (ROC) curves of the 2500 trained models, for Bai 2022 and Fraietta data, and (B) PR and PR_{TD} samples (not used in model training) classifications mixed between 0 and 1, according to an embodiment.

FIG. 14 shows PKPD response depending on initial tumor burden and CAR-T dose in CR, according to an embodiment.

FIG. 15 shows goodness-of-fit plots to Abecma®, according to an embodiment.

FIG. 16A shows a flowchart of a method for training a machine learning classifier, according to an embodiment.

FIG. 16B shows a flowchart of a method for training a machine learning classifier, according to an embodiment.

FIG. 17 shows a flowchart of a method for predicting a clinical outcome of a patient in response to a cell therapy treatment, according to an embodiment.

FIG. 18 shows a flowchart of a method for generating a predicted clinical outcome, according to an embodiment.

FIG. 19 shows a flowchart of a method for predicting a clinical outcome of a patient in response to a cell therapy, according to an embodiment.

FIG. 20 shows a flowchart of a method for administering a cell therapy to a patient, according to an embodiment.

FIG. 21 shows a flowchart of a method for producing a cell therapy product, according to an embodiment.

FIG. 22 shows a flowchart of a method for determining a patient-specific dosage of cell therapy to be administered, according to an embodiment.

FIG. 23 shows a block diagram of a system for performing one or more concepts/methods discussed herein, according to an embodiment.

FIGS. 24A-24B show a list of model parameters, units, and lower and upper bounds used in a particle swarm optimization algorithm, according to an embodiment.

FIG. 25 shows 12 CR (complete responder) example parameter sets for the parameters of FIGS. 24A-24B, according to an embodiment.

FIG. 26 shows 12 PR (partial responder) example parameter sets for the parameters of FIGS. 24A-24B, according to an embodiment.

FIG. 27 shows 12 NR (non responder) example parameter sets for the parameters of FIGS. 24A-24B, according to an embodiment.

FIG. 28 shows a depiction of the structures of original model and potential variants, according to an embodiment.

FIG. 29 shows fitting accuracy of a model against five structural variants.

FIG. 30 shows model fitting to pre-clinical CD19-CAR-T data, according to an embodiment.

FIG. 31 shows model fitting to pre-clinical BCMA-CAR-T data, according to an embodiment.

FIG. 32 shows simulated frequencies of memory (T_{M}), effector (T_{E}), and exhausted (Tx) CAR-T cells for CR, PR, and NR patient groups shown in FIG. 1, according to an embodiment.

FIG. 33 shows pairwise Pearson correlation coefficients between Thymic Atlas cell population gene signatures computed using ssGSEA scores from Fraietta et al. RNAseq data, according to an embodiment.

FIG. 34 shows single cell RNA sequencing of twelve pre-infusion CAR-T products classified by response, according to an embodiment.

FIG. 35 shows comparative pharmacokinetics of Kymriah® in B-ALL, the model described herein of Kymriah® in CLL, and Yescarta® in LCBCL, according to an embodiment.

FIG. 36 shows cmax/tumor burden vs. tumor response simulations for Abecma®, according to an embodiment.

FIG. 37 shows single cell RNA sequencing of two pre-infusion CAR-T products separate by response, according to an embodiment.

FIG. 38 shows Kymriah® response predictions in CLL using a ssGSEA-based classifier, according to an embodiment.

FIG. 39 shows (A) Cluster size and (B) mean within-cluster correlation distribution based on a variable number of separate clusters calculated for the 864 gene signatures differentially enriched between CR and NR groups, according to an embodiment.

FIG. 40 shows cell-intrinsic defects associated with non-durable response, according to an embodiment.

FIG. 41 shows CD19-CART response predictions, according to an embodiment.

FIG. 42 shows model fitting results based on the hypothesis that the only distinguishing feature between CR, PR, and NR populations is the fraction of memory T and exhausted T cells in the CAR-T infusion product, according to an embodiment.

FIG. 43 shows simulated pharmacokinetic and tumor dynamic responses to increasing cell doses of pure memory cell populations from CR, PR, and NR population models, according to an embodiment.

FIG. 44 shows T cell phenotyping of CAR-T infusion products. according to an embodiment.

FIG. 45 shows transcriptome classifier performance as compared to null and random pathway models. according to an embodiment.

### DETAILED DESCRIPTION OF THE DISCLOSURE

In some implementations. as used herein cell therapy can include any suitable cell therapy. such as adoptive cell therapy and cellular immunotherapy. Cellular immunotherapy is a form of treatment that uses the cells of our immune system to eliminate cancer cells or other unwanted cells. Some of these approaches involve directly isolating our own immune cells and simply expanding their numbers. whereas others involve genetically engineering our immune cells (via gene therapy) to enhance their cancer-fighting capabilities. Our immune system is capable of recognizing and eliminating cells that have become infected or damaged as well as those that have become cancerous. In the case of cancer, immune cells known as killer T cells can be powerful against cancer, due to their ability to bind to markers known as antigens on the surface of cancer cells. Cellular immunotherapies take advantage of this natural ability and can be deployed in different ways. Illustrative types of cell therapies include, for example, Chimeric Antigen Receptor T cell therapy (CAR-T cell therapy). Tumor-Infiltrating Lymphocyte (TIL) therapy, engineered T Cell Receptor (TCR) therapy and Natural Killer (NK) cell therapy. The apparatus and methods of the present disclosure are specifically exemplified with respect to CAR-T cell therapy. However, it would be understood that the present disclosure can also be readily applied to other cell therapies.

As used herein, a clinical outcome can include. for example. complete response. partial response. non-response. In some implementations. response category definitions for a clinical outcome are disease indication-specific, and refer to, for example, tumor burden measured at a defined time following administration of therapy.

Cmax (maximal concentration) is the maximally observed abundance of CAR-T cells in circulation following administration, sometimes occurring 1 week to 1 month post-dose. CAR-T cells may be quantified using, for example, bioluminescent imaging (BLI). PCR for the CAR transgene (counts/ug genomic DNA), or by flow cytometry for CAR expression (% circulating T-cells). AUC is the Area Under the Curve computed from the pharmacokinetic time-course.

Illustrative forms of gene expression data that can be obtained and used according to the present disclosure include, for example, RNAseq, RNA sequencing data; scRNAseq, single cell RNA sequencing data; GSEA, Gene Set Enrichment Analysis; and ssGSEA, single sample Gene Set Enrichment Analysis.

Machine learning classifiers can be, for example, a classifier used to automatically categorize data and can include, for example, logistic regression, multinomial logistic regression, decision tree, perceptron, support vector machines, K-nearest neighbor, Naïve Bayes, random forest, etc.

As used herein a pathway or biological pathway can be a set of genes known to be involved in a defined intracellular biochemical mechanism.

Pharmacokinetic/pharmacodynamic models, can, in some implementations, integrate a pharmacokinetic and pharmacodynamic model component into a set of mathematical expressions to describe the dynamics of the drug and/or physiological components in response to an administered dose of the drug.

Mathematical models of T cell-tumor interactions can be adapted to describe various aspects of CAR-T pharmacology-antigen binding. intercellular signaling cytokine release, tissue distribution and/or competition with host T cells for immune system reconstitution. Some known models, however, fail to adequately define what limits cell expansion, nor what underlies the wide variability in exposure and tumor response observed between patients. Furthermore, such known models are slow and inefficient due to the usage of large, non-optimal datasets.

Insights can be gleaned by examining the T cell dynamics in response to viral infection. Upon viral antigen encounter, antigen-specific T cells clonally expand and differentiate into cytotoxic effectors, which clear infected cells. Following elimination of the pathogen, effector cells undergo a precipitous contraction phase, and a small percentage survive to form long-term memory T cells capable of self-renewal and recall responses. However, if the infection fails to resolve, chronic antigen stimulation leads to T cell exhaustion, wherein remanent T cells lose the ability to produce cytokines, kill target cells or proliferate in response to antigen.

In an embodiment, an analogous process underlies the pharmacology of CAR-Ts. Some concepts discussed herein are related to using a mathematical model of T cell differentiation control, wherein an antigen-driven toggle switch regulates cell fate transitions between memory, effector, and exhausted T cells. The model is capable of quantitatively describing pharmacokinetic and tumor dynamic data from multiple clinical trials and deconvolutes cell- and host-intrinsic sources of inter-patient variability. These mathematical results can be confirmed via analysis of bulk and single cell RNAseq profiles of CAR-T products and find that the pre-infusion transcriptome is predictive of response. The model predicts, de novo, clinical variance in exposure, covariates of response and the underlying biological mechanisms.

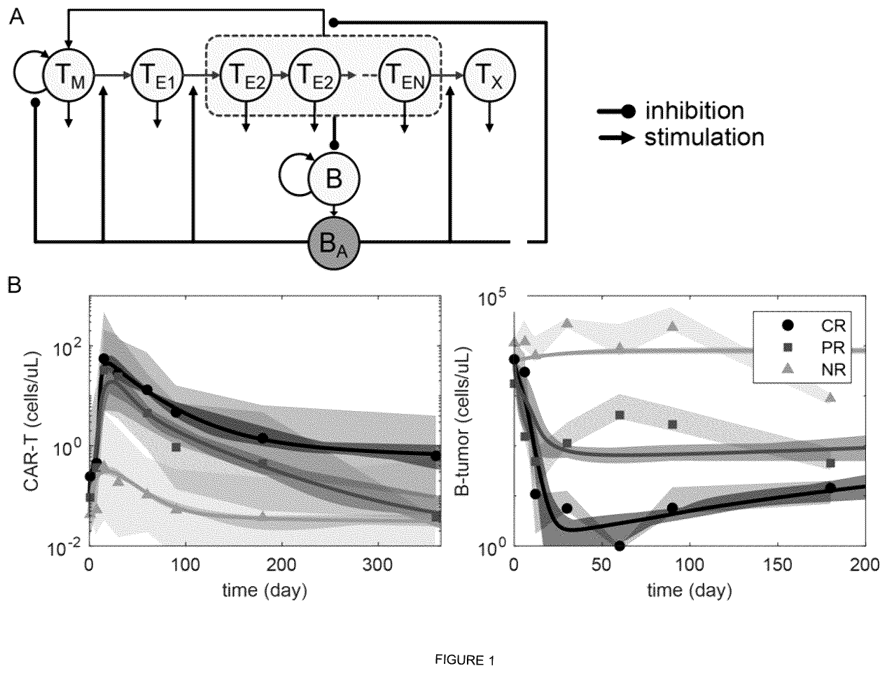

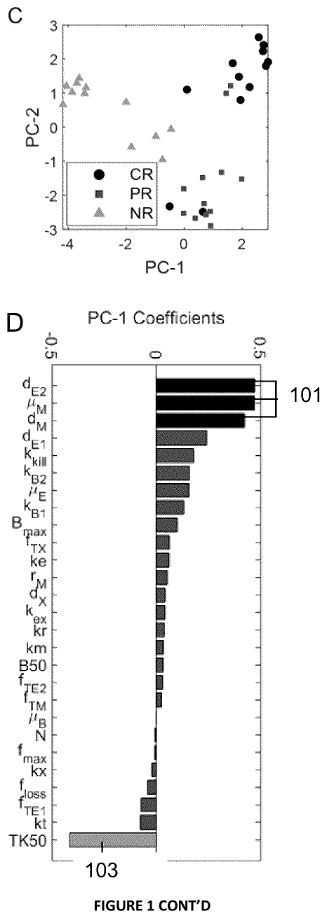

FIG. 1 shows an antigen toggle-switch model of T cell regulation that quantitatively describes PKPD behavior of complete responder (CR). partial responder (PR), and non-responder (NR) patient population response to Kymriah R in Chronic Lymphocytic leukemia (CLL). according to an embodiment. A Depiction of the model structure. comprising three populations of T cells: T memory cells (T_{M}). T effector cells (T_{E1} and T_{E2}) and exhausted T cells (Tₓ). and B cell tumors (B). Tumor cells express B cell antigen (B_{A}) which stimulates T cell proliferation and differentiation, and inhibits the formation of T memory cells. B The model structure fit to known PKPD profiles separated by response category (CR/PR/NR) using particle swarm optimization (PSO). Model fits (curves: mean of 12 parameter sets: dark shaded areas: middle 90%) agree with both CAR-T and B-cell tumor dynamics over time (dots: mean data: light shaded areas: range of data) for each of the three prototypic populations. C PCA plot of the logarithm of the best fitting parameters colored by population. Principal component one (PC-1) captures 35.3% of the variability and principal component two (PC-2) captures 21.7% of the variability. D Sorted PC-1 coefficients suggest that TK50 (bar 103) and d_{E2}. UM and d_{M} (bars 101) are the largest sources of variation between CR and NR populations. These parameters correspond to cytotoxic potency. effector cell death rate. memory cell proliferation and death rates, respectively.

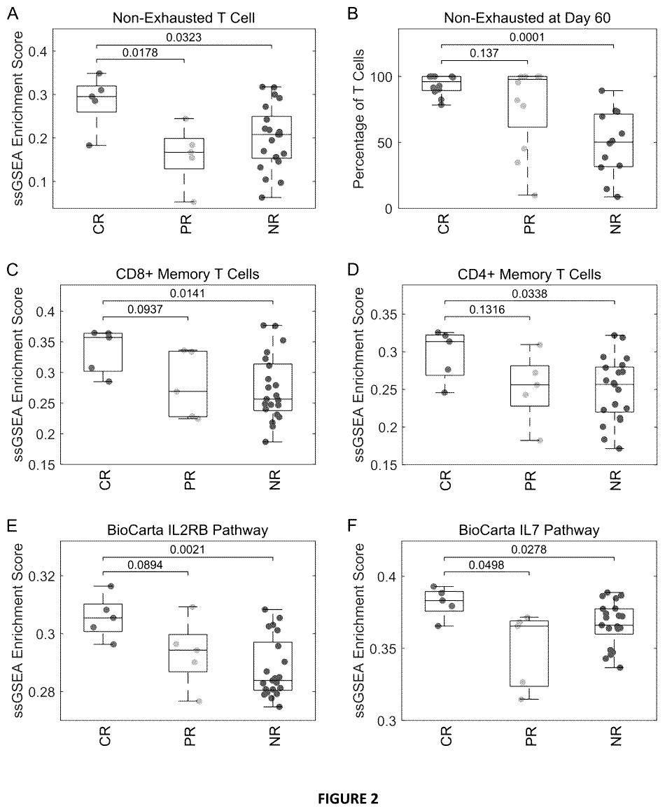

FIG. 2 shows single-sample Gene Set Enrichment Analysis (ssGSEA) estimates of the activity of signaling pathways and enrichment of cell populations in CAR-Ts, separated by response. according to an embodiment. A, C-F ssGSEA reveals differences in cell populations and signaling pathways between populations for selected cell signatures and signaling pathways (panel titles). Differences between populations were assessed using an unequal variances/-test (p-values shown). B Using the 12 best fitting parameter sets for each population and model simulations, the percentage of the T cell population at day 60 that is non-exhausted was calculated. The median non-exhausted T cell population at day 60 (over the 12 parameter sets) is near 100% for both CR and PR populations while the median is approximately 50% for the NR population. Differences between populations were assessed using an unequal variances t-test (p-values shown).

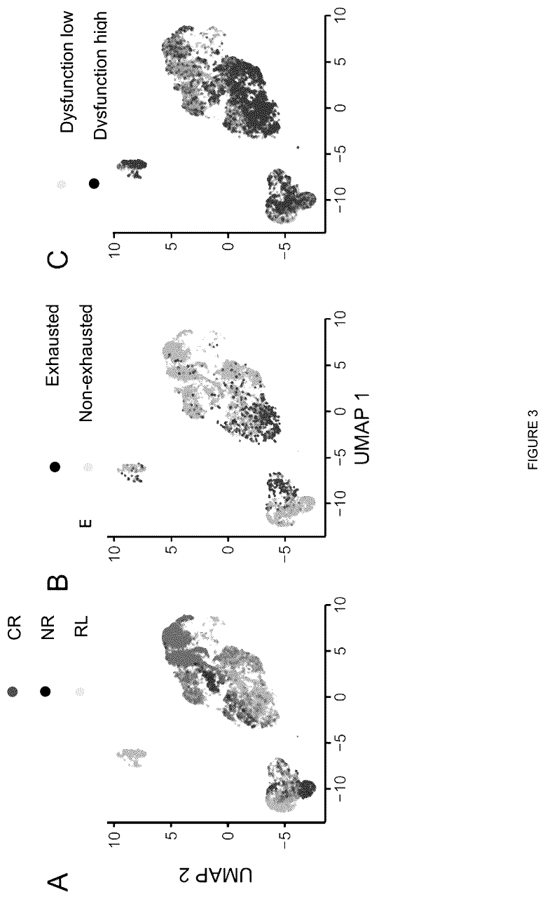

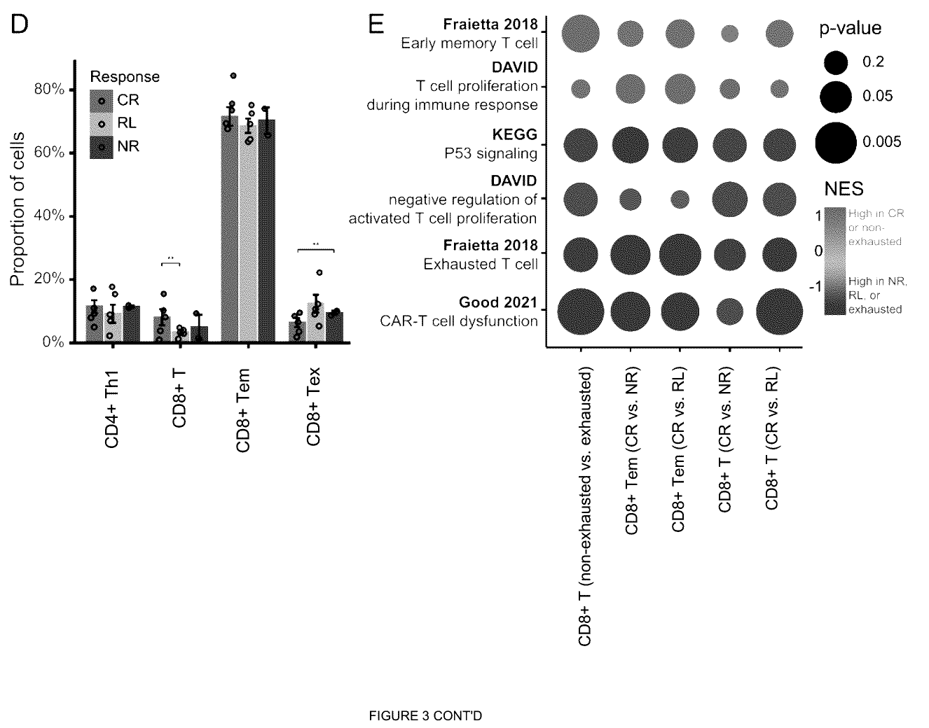

FIG. 3 shows single cell RNA sequencing of twelve pre-infusion CAR-T products classified by response, according to an embodiment. A UMAP (Uniform Manifold Approximation and Projection) projection of cells annotated by response category. CR: complete response, RL: relapsed, NR, non-response. B UMAP projection of cells annotated as exhausted using ProjectTILs (see, e.g., Andreatta, M. et al. Interpretation of T cell states from single-cell transcriptomics data using reference atlases. Nat Commun 12. 2965 (2021). the contents of which are incorporated by reference herein in its entirety) C UMAP projection of cells annotated for high (above mean) or low (below mean) CAR-T cell dysfunction signature from Good et al. (see, e.g., Good, C. R. et al. An NK-like CAR T cell transition in CAR T cell dysfunction. Cell (2021) doi:10.1016/j.cell.2021.11.016., the contents of which are incorporated by reference herein in its entirety) D Cell type frequencies derived using ProjectTILs for the CR, RL, and NR groups. Only cell types for which there is at least 5% representation in the sample are shown. Data is shown as mean+−standard error, with individual sample frequencies overlaid as dots. Significance is shown for significant comparisons between CR and NR according to a Wilcoxon rank-sum test (p<=0.05). E Per-cell type GSEA for selected pathways, which show significant enrichment (p<=0.05) in at least one cell type. A positive normalized enrichment score (NES) indicates higher enrichment in CR/non-exhausted cells. Tₑₘ, effector memory T cells: Th1. Type 1 helper T cells: Tₑₓ. exhausted T cells.

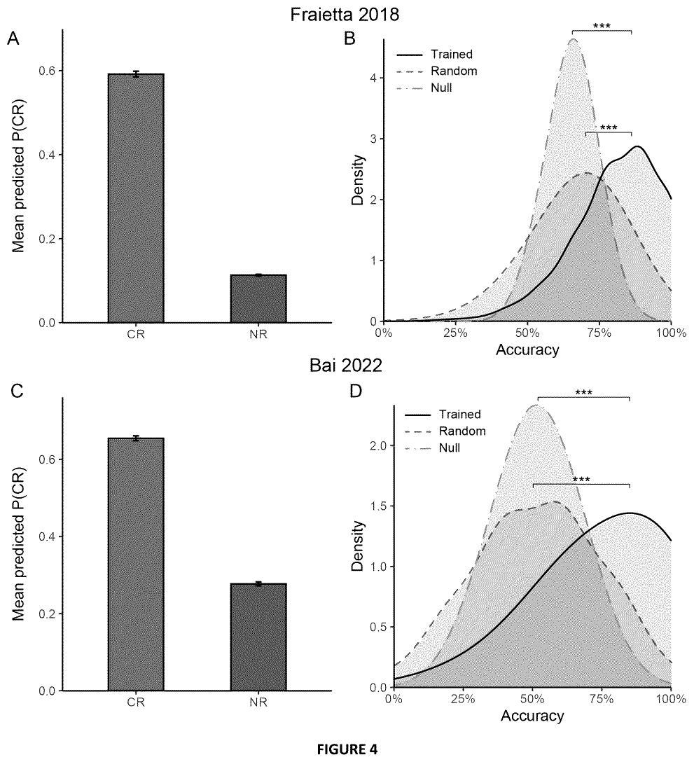

FIG. 4 shows that Kymriah R response can be accurately predicted using a ssGSEA-based classifier, according to an embodiment. A, C Trained classifier predictions for Fraietta data (see. e.g., Fraietta, J. A. et al. Determinants of response and resistance to CD19 chimeric antigen receptor (CAR) T cell therapy of chronic lymphocytic leukemia. Nat Med 24, 563-571(2018). the contents of which are incorporated by reference herein in its entirety) (A) and pseudobulked Bai 2022 data (see, e.g., Bai, Z. et al. Single-cell antigen-specific landscape of Sci Adv 8. (2022), the contents of which are incorporated by reference herein in its entirety) (C) from 2500 iterations on the test split of the data. Predicted probability of CR (p(CR)) is plotted as the mean across 2500 models±standard error. B, D Distribution of model accuracy for the trained model on Fraietta data (B) and pseudobulked Bai 2022 data (D) compared to two test cases: a null model. which predicts response or non-response according to the proportion of each label in the dataset, and a random model. which selects random pathways to use as features rather than our derived set of 30 pathways. Significance is shown for comparisons with p≤10⁻⁸, Wilcoxon rank-sum test.

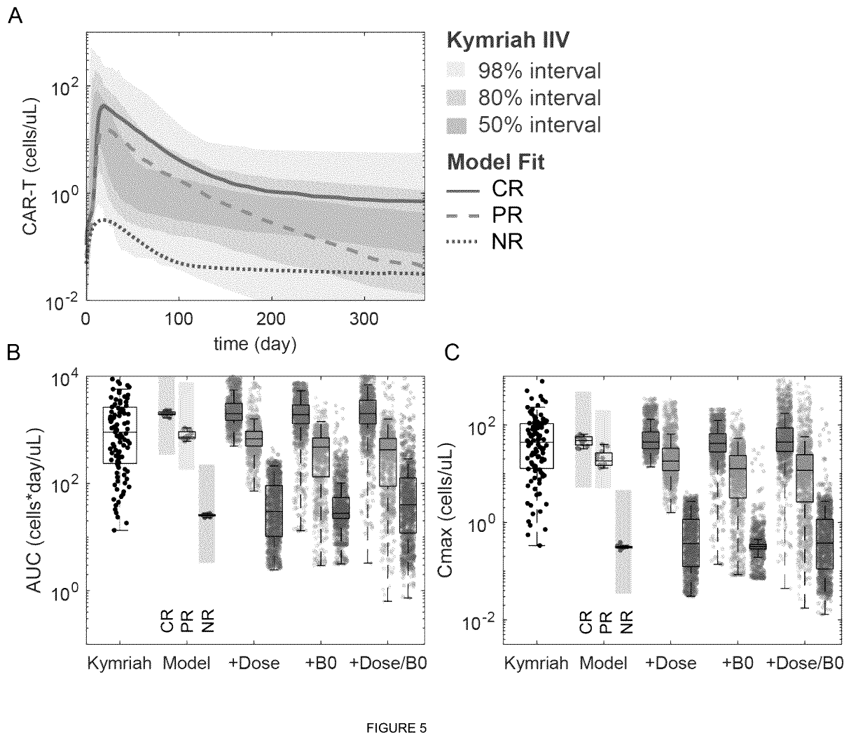

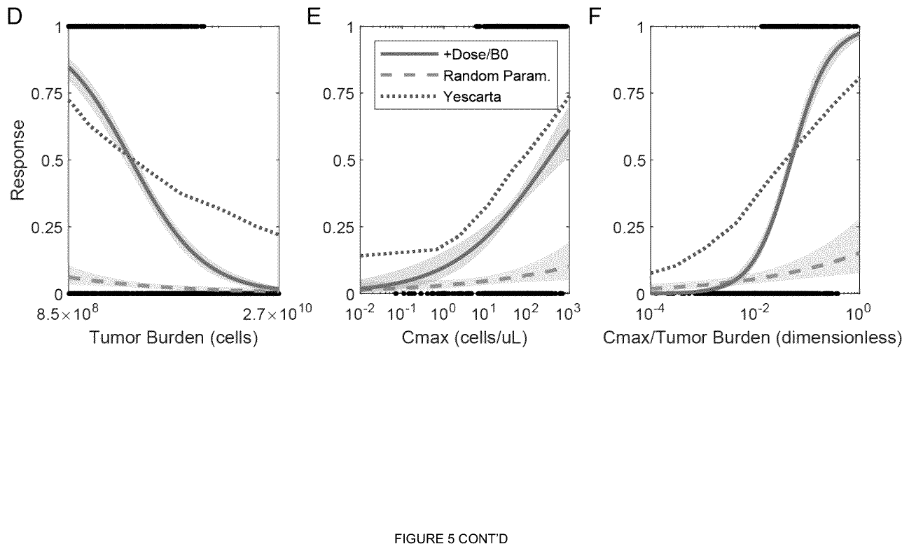

FIG. 5 shows that clinical variability in dose, tumor burden. and CR/PR/NR pharmacological archetype account for population variance in exposure to Kymriah R. and predict clinical covariates of response to Yescarta R. according to an embodiment. A Shaded areas show the clinical variability in exposure to Kymriah R with median model simulations overlaid for the CR, PR, and NR populations. B CAR-T AUC distributions. The first boxplot. labelled “Kymriah”. shows the distribution in AUC obtained from 1000 simulations of the clinical PK model (each black dot corresponds to a percentile of the AUC distribution). The group of boxplots labelled “Model” show the AUC distribution obtained for the 12 best fitting parameter sets for each population, including CR, P35 std). The group of boxplots labelled “+Dose” show the AUC distributions for each population when doses are randomized within reported ranges in the virtual population: “+B0” show the distributions when initial tumor burdens are randomized: and “+Dose/BO” show the distribution when both dose and initial tumor burdens are randomized. C Cmax distributions plotted as in A. D-F Response to treatment was defined as tumor AUC less than 10,000 cells*day/μL and evaluated whether each patient in the virtual CR population with randomized doses and tumor burdens (+Dose/B0) exhibited a response (black binary data points). Logistic regression (shown as solid lines with 95% confidence intervals and labeled “+Dose/B0”) with respect to the tumor burden (D). Cmax (E), or the quotient of Cmax and tumor burden (F), reveals how each predicts response. As a control. uniform random sampling of parameter space (1000) parameter sets) does not exhibit these response relationships (dashed line labelled “Random Param.” and confidence intervals). The clinical covariates of response calculated using the virtual population have the same trends as published covariates of response to Yescarta® (dotted lined labelled “Yescarta”). Note that the covariates of response for Yescarta R have been linearly scaled to match the ranges in the virtual population for plotting.

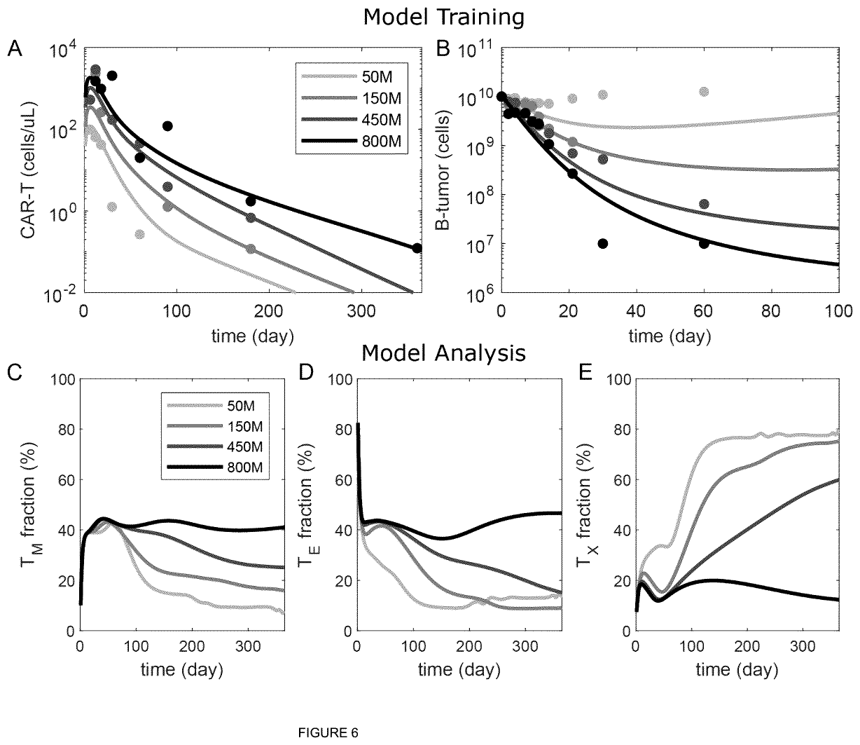

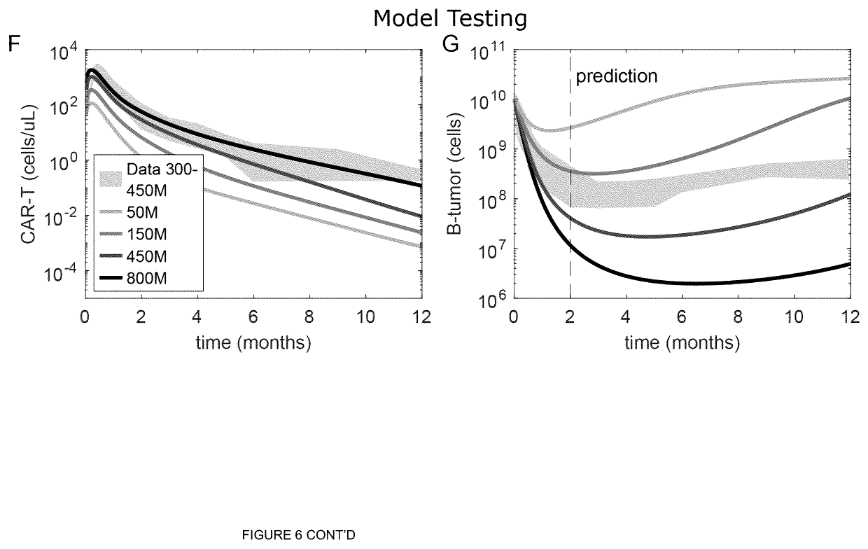

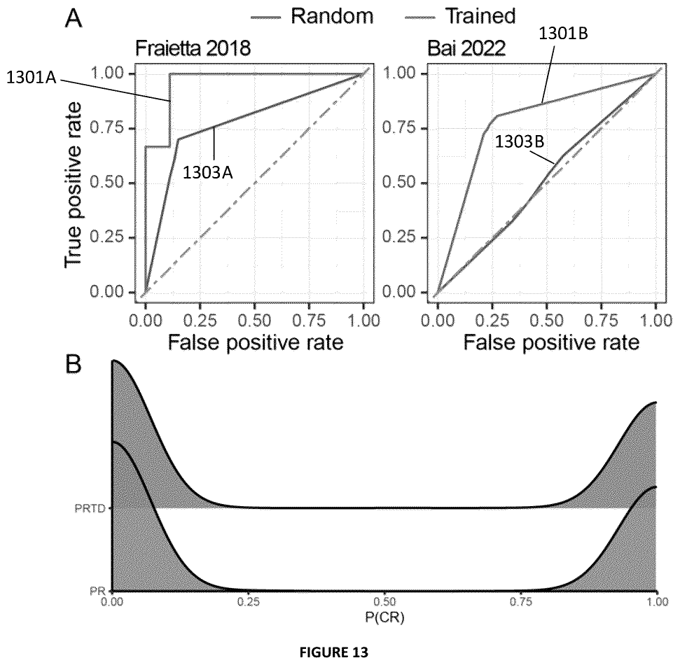

FIG. 6 shows model extension to Abecma® dose response. according to an embodiment. Model Training (A and B): the toggle-switch model fit to phase I dose-response data and observed desirable fits, with Pearson's linear correlation coefficients from the Goodness-of-Fit plots (FIG. 13) of 0.59 for the CAR-T cells and 0.74 for the tumor. Model Analysis (C, D. and E): comparison of the fraction of the total T cell population across doses in the memory. effector. and exhausted groups by plotting the mean over the best fitting parameter sets from simulations. For low doses. the T cell population becomes mostly exhausted. while for high doses. the population of memory and effector cells persists for longer. Model Testing (F and G): comparisons of predictive simulations at two doses with the data reported in known studies (150)-450M cell doses). The tumor dynamics out to one year fall within the bounds predicted for the 150-450M cell doses.

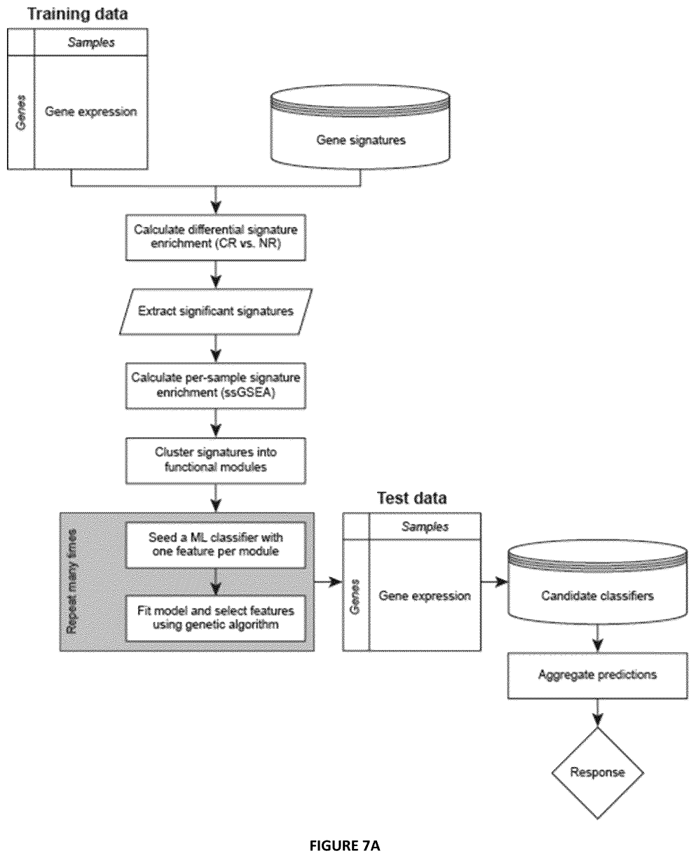

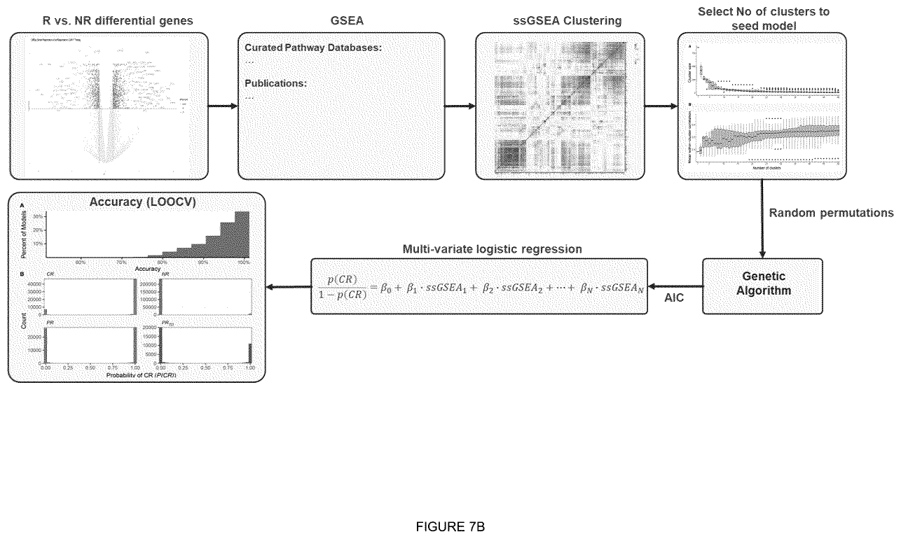

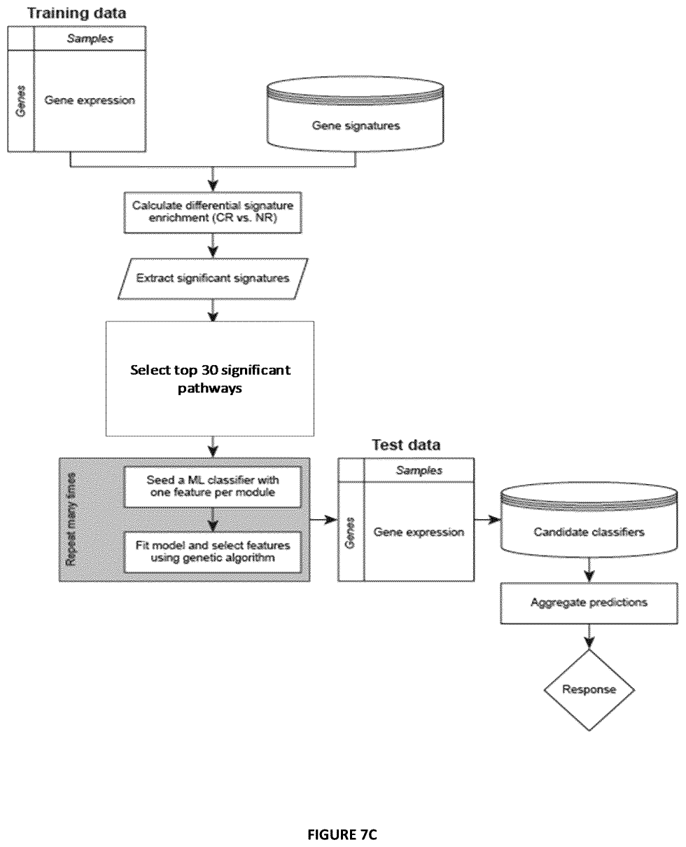

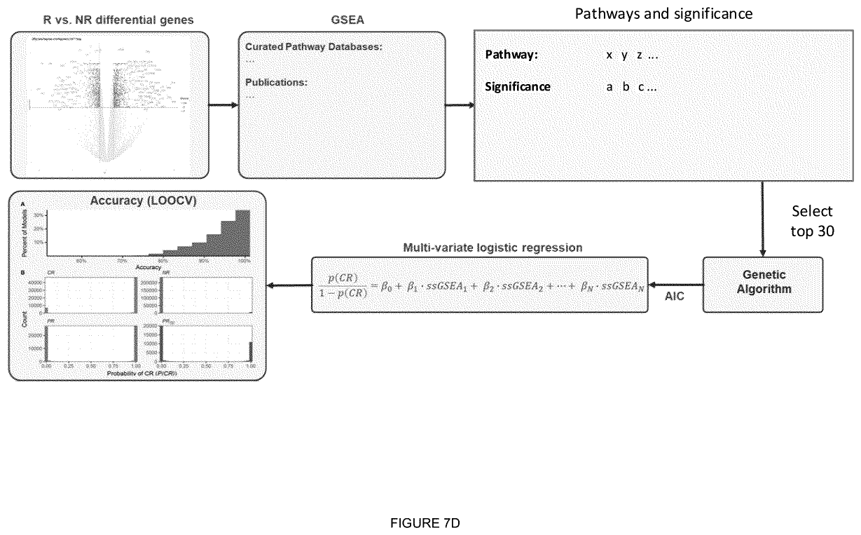

FIG. 7A shows a flowchart of response classifier workflow, according to an embodiment. Using a database of gene signatures (e.g., DAVID, PROGENy, Hallmark, etc.) and a reference gene expression dataset, GSEA can be performed to discover a subset of gene signatures which are statistically significantly enriched in either the NR or CR groups. ssGSEA scores can then be calculated for that subset of signatures. hierarchically clustered into 26 modules, and seeded into a machine learning classifier with one term per module to predict clinical response. Each classification model can be trained using a genetic algorithm until convergence, and to ensure robustness, many models were fitted by seeding with different starting terms.

FIG. 7B shows a pictorial representation of the response classifier workflow. showing example data at each step in the pipeline using a process similar to that discussed with respect to FIG. 7A. according to an embodiment. Interpretation of differential expression analysis. shown as a volcano plot. through GSEA allows for the selection of a subset of gene signatures. ssGSEA scores can then be computed for this subset, and for terms are clustered to maximize and/or increase intercluster correlation while simultaneously minimizing and/or reducing the average cluster size (pictured as a heatmap and barplots). Machine learning models can be trained and evaluated, and example results showing the cross-validated accuracy and distribution of response predictions are shown.

FIG. 7C shows a flowchart of response classifier workflow. according to an embodiment. Using a database of gene signatures (e.g., DAVID, PROGENY, Hallmark, etc.) and a reference gene expression dataset. GSEA can be performed to discover a subset of gene signatures that are statistically significantly enriched in either the NR or CR groups. The top X (e.g., 10, 20, 30, 40, etc.) significantly enriched gene signatures from the subset of gene signatures are selected/identified for use as feature vectors, and seeded into a machine learning classifier with one term per module to predict clinical response. Each classification model can be trained using a genetic algorithm until convergence, and to ensure robustness, many models were fitted by seeding with different starting terms. In some implementations, unlike FIGS. 7A and 7B. clustering is not performed. In some implementations, pathways were ranked by false discover rate (FDR)-adjusted p-value (p), based on difference between the CR vs. NR populations. For example, a p-value cut-off threshold of p<0.05 can be set. In at least one instance. a p-value cut-off threshold of p<0.05 resulted in a set of 30 top pathways. Note. however. that any suitable p-value threshold can be used (e.g., 0.01. 0.02, etc.).

FIG. 7D shows a pictorial representation of the response classifier workflow. showing example data at each step in the pipeline using a process similar to that discussed with respect to FIG. 7C. according to an embodiment. Interpretation of differential expression analysis. shown as a volcano plot, through GSEA allows for the selection of a subset of gene signatures.). The top X (e.g., 10, 20, 30, 40, etc.) significantly enriched gene signatures from the subset of gene signatures are selected/identified for use as features vectors, and input into a genetic algorithm and/or model. Feature vectors (e.g., top 30 pathways) are input to the genetic algorithm and/or model to run feature selection. This output pathways (e.g., 2-6 pathways). which are then fed into the multi-variate logistic regression for model fitting. If the genetic algorithm and/or model is stochastic, the features the genetic algorithm and/or model selects can change each time it is run (e.g., hence the 2,500 iterations to create a distribution of models). Machine learning models can be trained and evaluated, and example results showing the cross-validated accuracy and distribution of response predictions are shown.

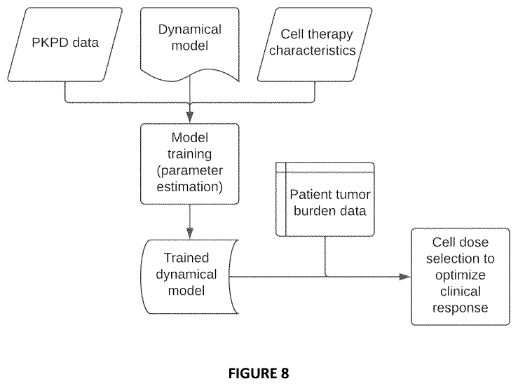

FIG. 8 shows a PKPD model workflow; according to an embodiment. A mechanism-based dynamical model integrating cell therapy characteristics is trained on PKPD data (e.g., CAR-T cells and tumor cells) to accurately predict dynamics. Given a patient tumor burden. the trained model can be used to select a dose of cell therapy that optimizes clinical response.

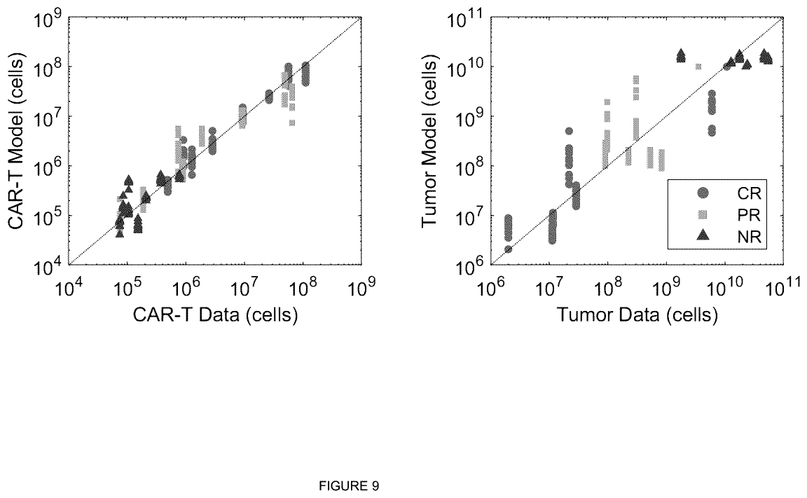

FIG. 9 shows goodness-of-fit plots for Kymriah® model fitting in FIG. 1, according to an embodiment.

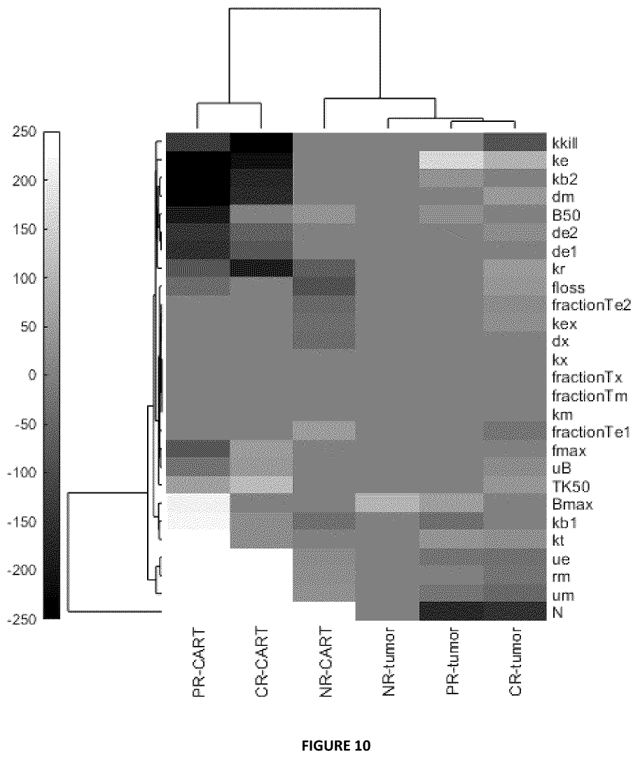

FIG. 10 shows Local Parameter Sensitivity Analysis of CR/PR/NR populations. according to an embodiment. Local Parameter Sensitivity Coefficients (LPSC) were calculated for CR, PR, and NR populations using the AUC of T cells (aucT) and AUC of B cells (aucB) as outputs. with samples and outputs organized by agglomerative hierarchical clustering.

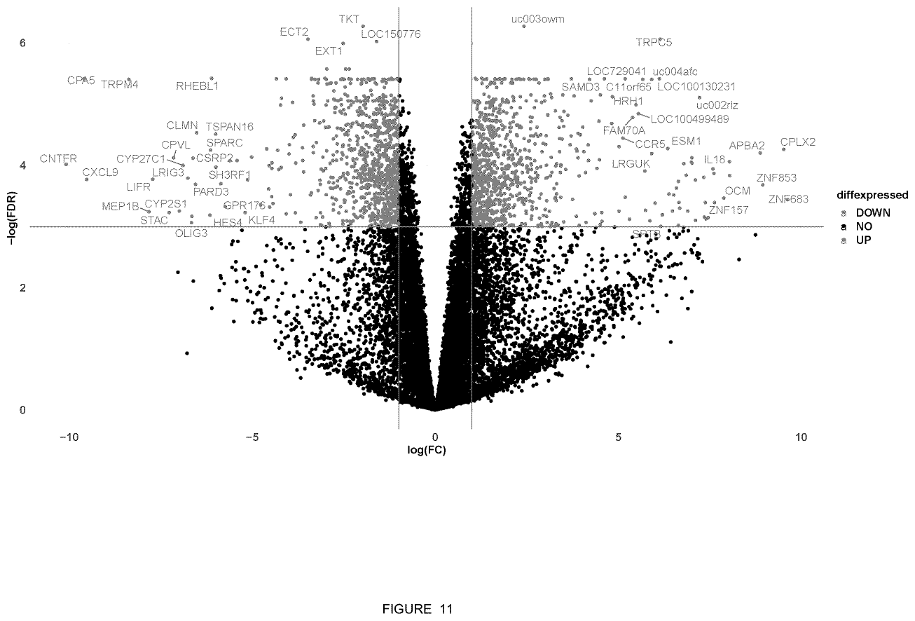

FIG. 11 shows a volcano plot of differentially expressed genes between CR vs. NR groups. according to an embodiment.

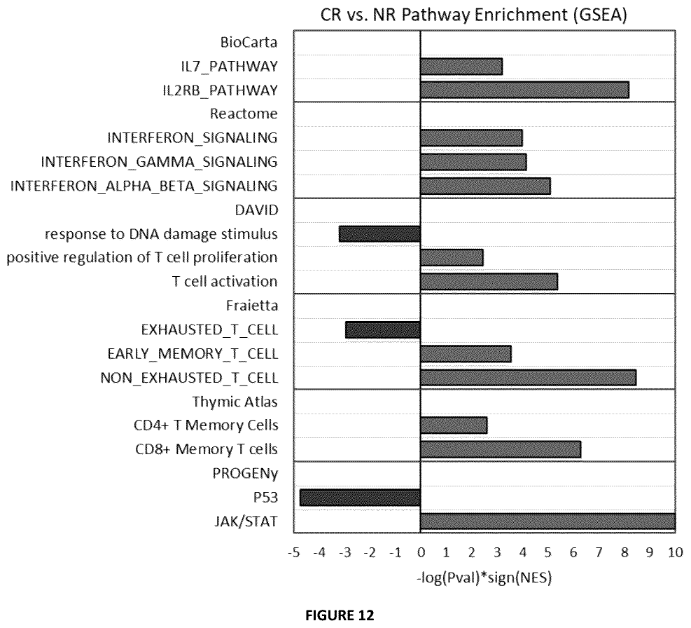

FIG. 12 shows select gene sets differentially enriched between CR vs. NR groups, according to an embodiment. Gene sets were derived from BioCarta, Reactome, DAVID, Fraitta et al., Thymic Cell Atlas and PROGENy, and represented as signed log 10(P-val).

FIG. 13 shows (A) Mean receiver operating characteristic (ROC) curves of the 2500 trained models, for Bai 2022 and Fraietta data. according to an embodiment. Lines 1301A, 1301B represent mean performance of the trained models using the selected pathways while lines 1303A, 1303B represent mean performance of the trained models using randomly selected pathways. (B) PR and PR_{TD} samples (not used in model training) classifications are mixed between 0 and 1.

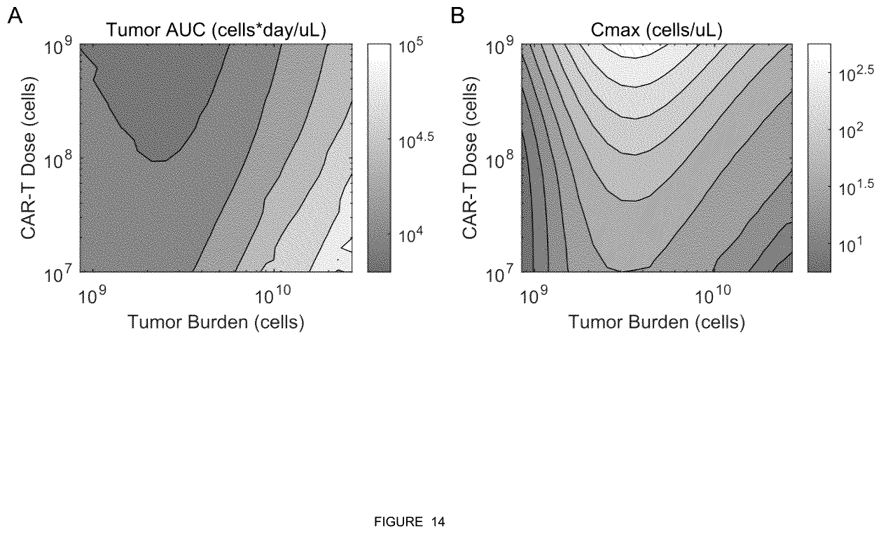

FIG. 14 shows that PKPD response depends on initial tumor burden and CAR-T dose in CR. according to an embodiment. Model simulations were performed across a grid of CAR-T dose. initial tumor burden, and parameter set in the CR population to determine the (A) average tumor AUC and (B) average CAR-T Cmax. Tumor AUC increases with initial tumor burden and decreases with initial CAR-T dose for CR parameters. Cmax exhibits a more complex relationship. peaking for intermediate tumor burdens and generally increasing with initial CAR-T dose.

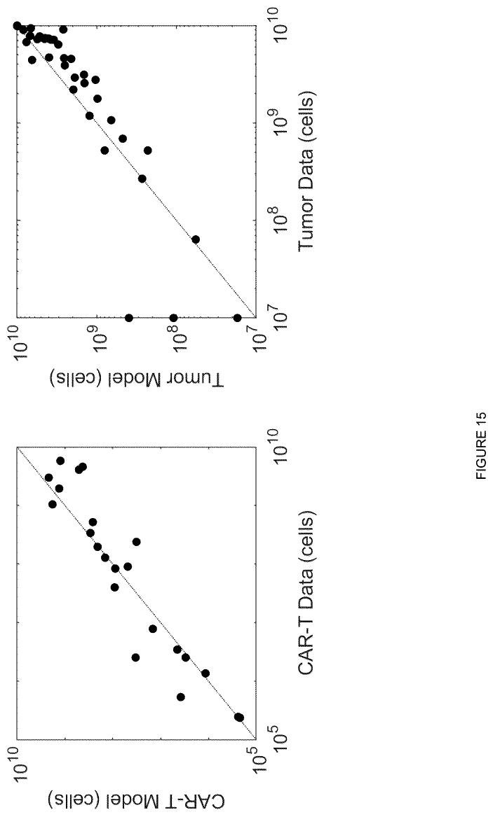

FIG. 15 shows goodness-of-fit plots to Abecma R, according to an embodiment. Data was fit simultaneously, with Pearson's linear correlation coefficient of 0.59 for CAR-T and 0.75 for tumors.

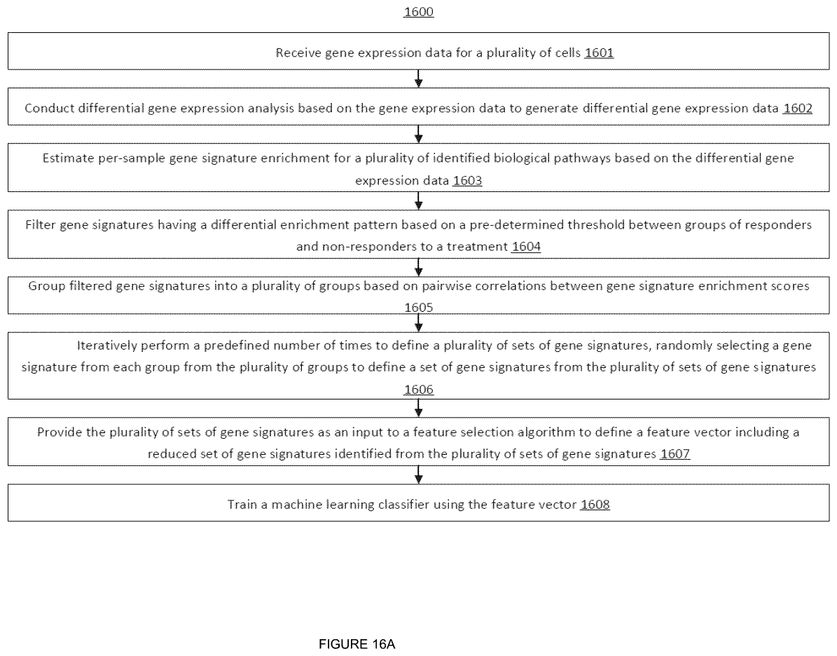

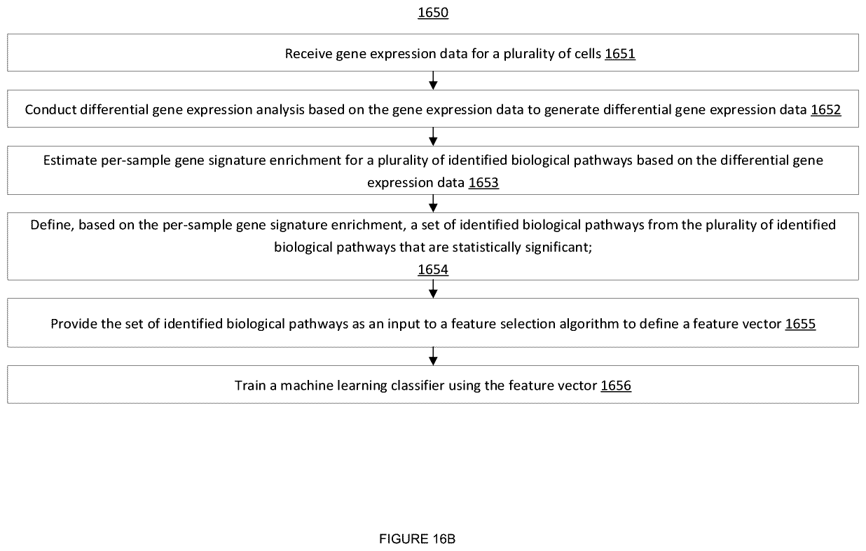

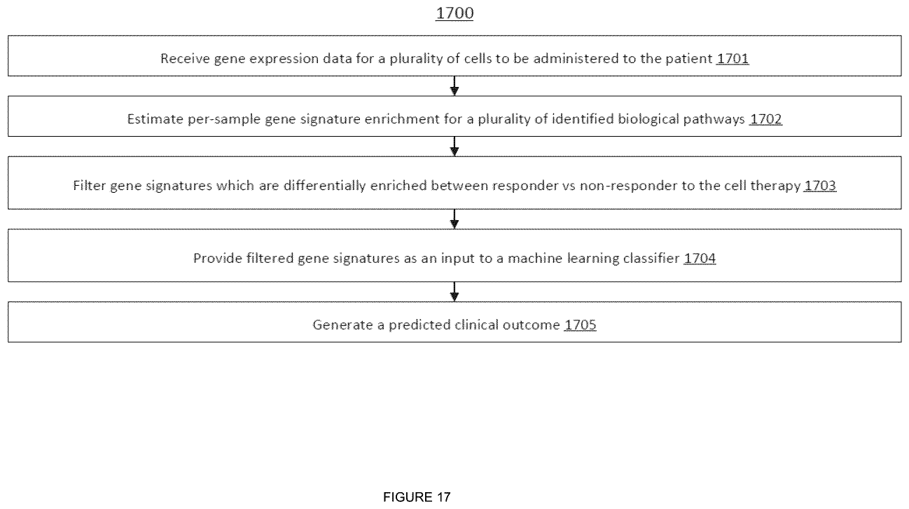

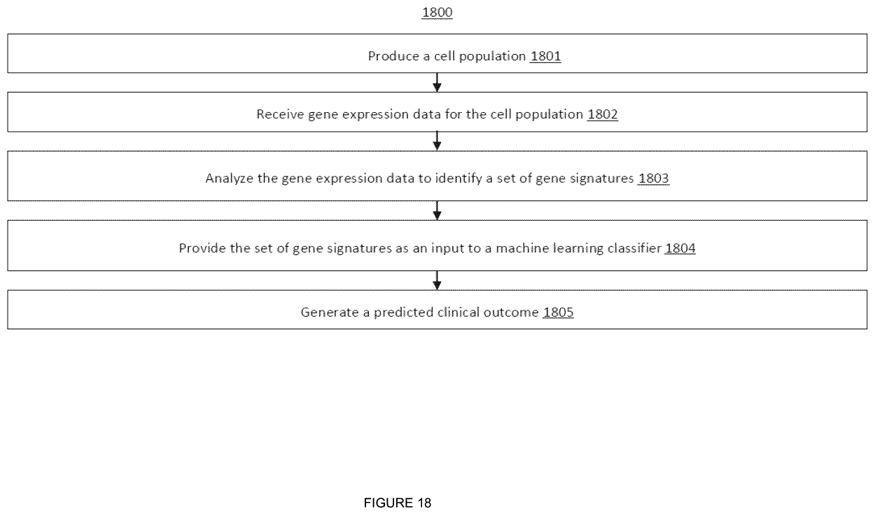

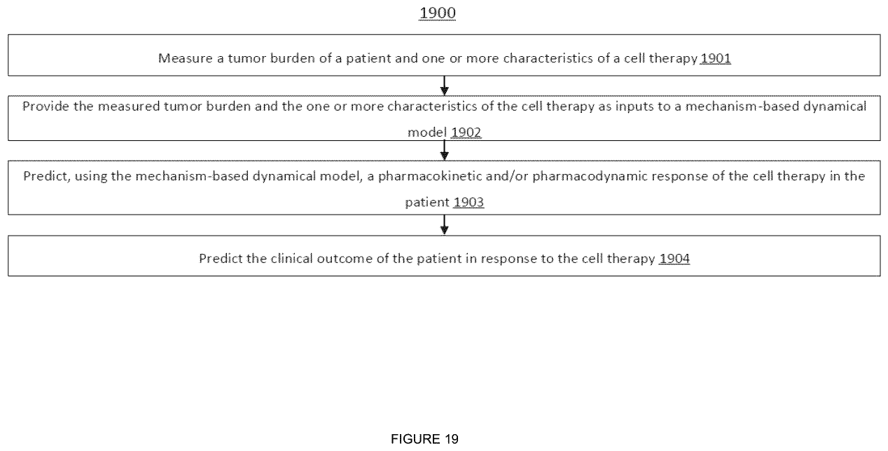

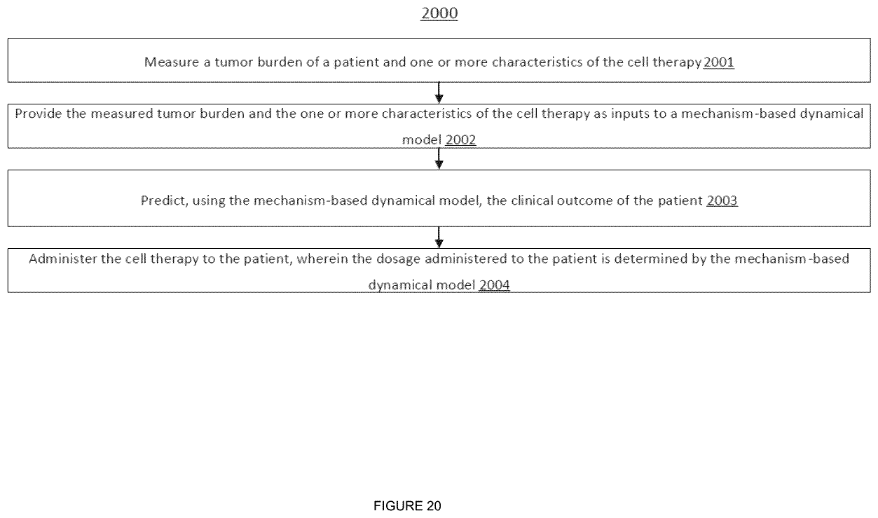

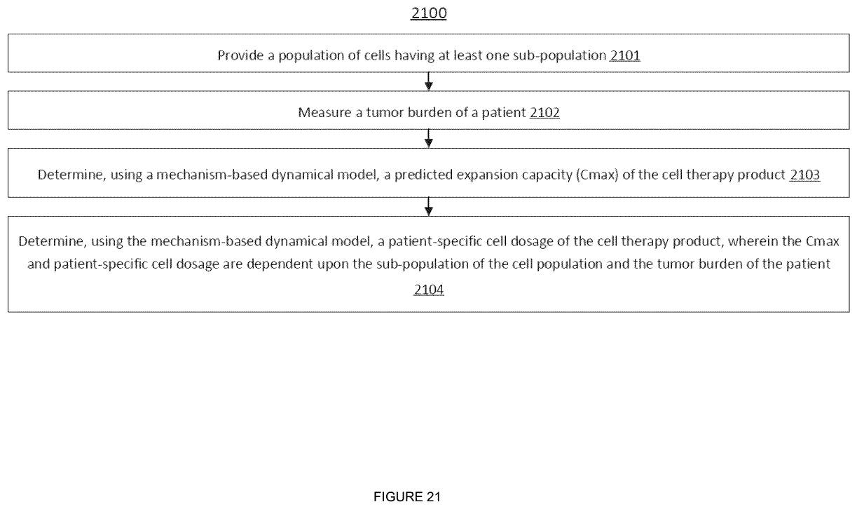

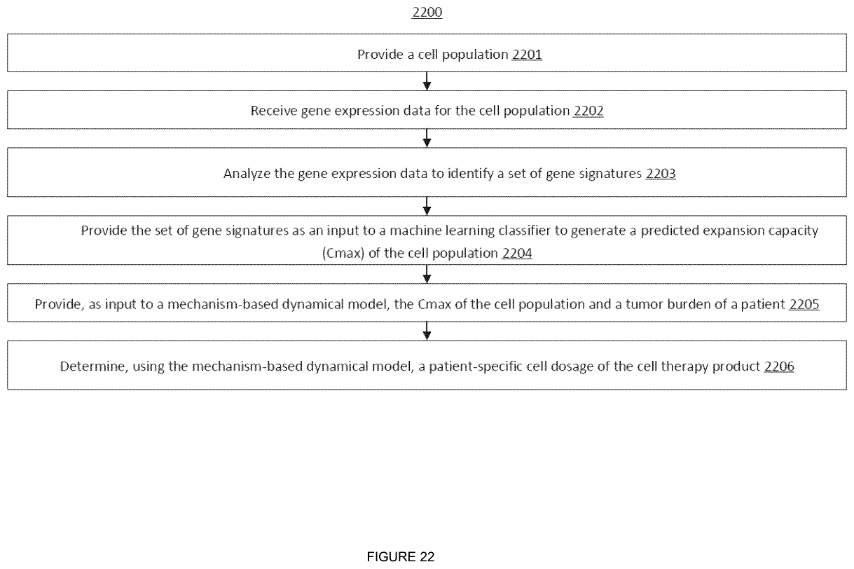

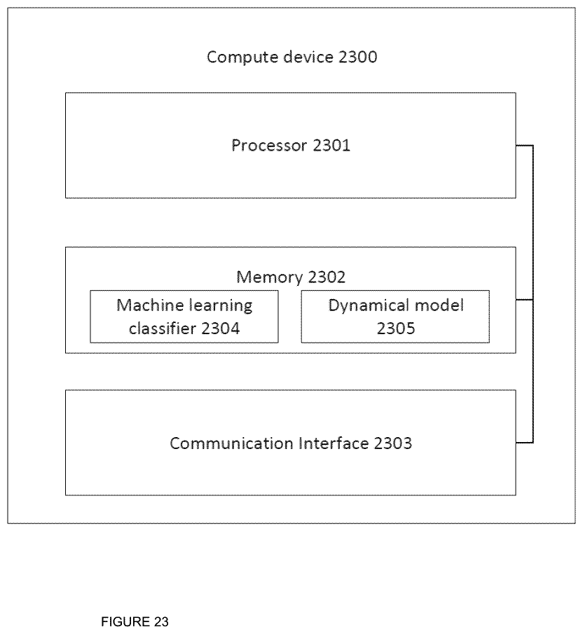

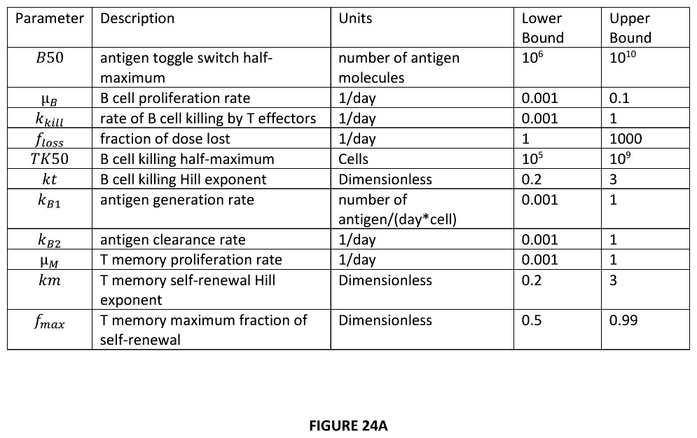

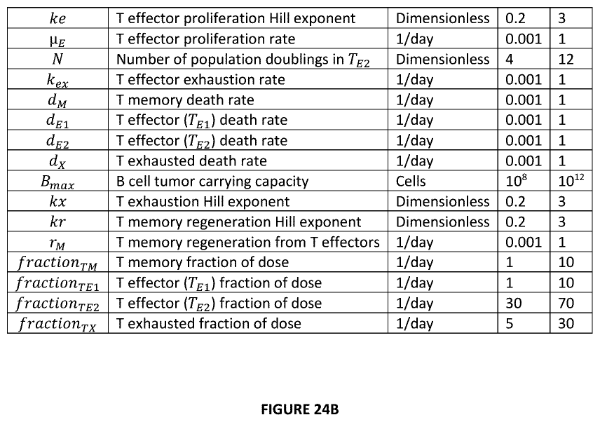

FIG. 16A shows a flowchart of a method for training a machine learning classifier. according to an embodiment. FIG. 16B shows a flowchart of a method for training a machine learning classifier, according to an embodiment. FIG. 17 shows a flowchart of a method for predicting a clinical outcome of a patient in response to a cell therapy treatment. according to an embodiment. FIG. 18 shows a flowchart of a method for generating a predicted clinical outcome. according to an embodiment. FIG. 19 shows a flowchart of a method for predicting a clinical outcome of a patient in response to a cell therapy. according to an embodiment. FIG. 20 shows a flowchart of a method for administering a cell therapy to a patient. according to an embodiment. FIG. 21 shows a flowchart of a method for producing a cell therapy product. according to an embodiment. FIG. 22 shows a flowchart of a method for determining a patient-specific dosage of cell therapy to be administered, according to an embodiment. FIG. 23 shows a block diagram of a system for performing one or more concepts/methods discussed herein, according to an embodiment. Additional details related to FIGS. 16A-23 are discussed below:

FIGS. 24A-24B show a list of model parameters, units, and lower and upper bounds used in a particle swarm optimization algorithm, according to an embodiment.

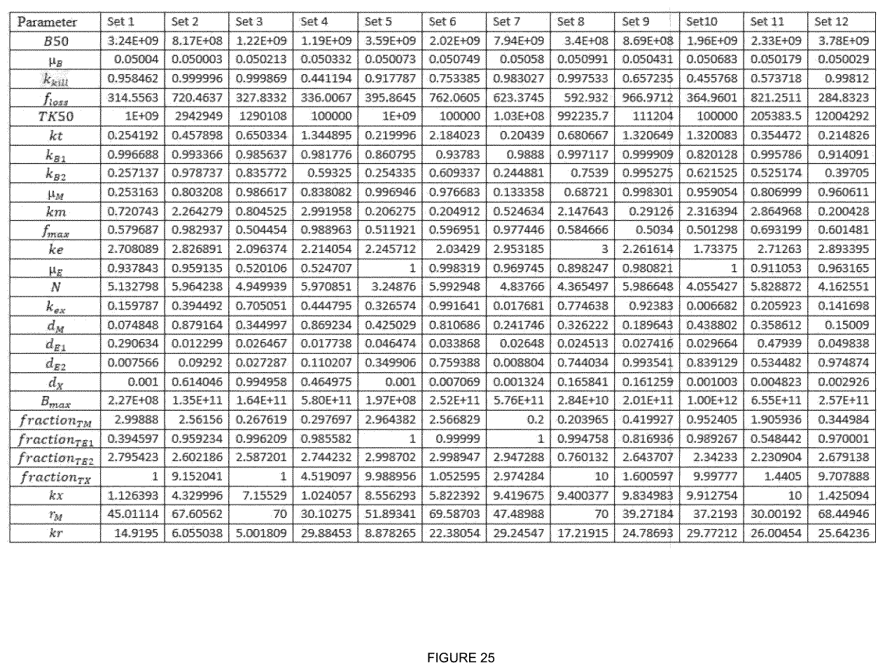

FIG. 25 shows 12 CR (complete responder) example parameter sets for the parameters of FIGS. 24A-24B, according to an embodiment.

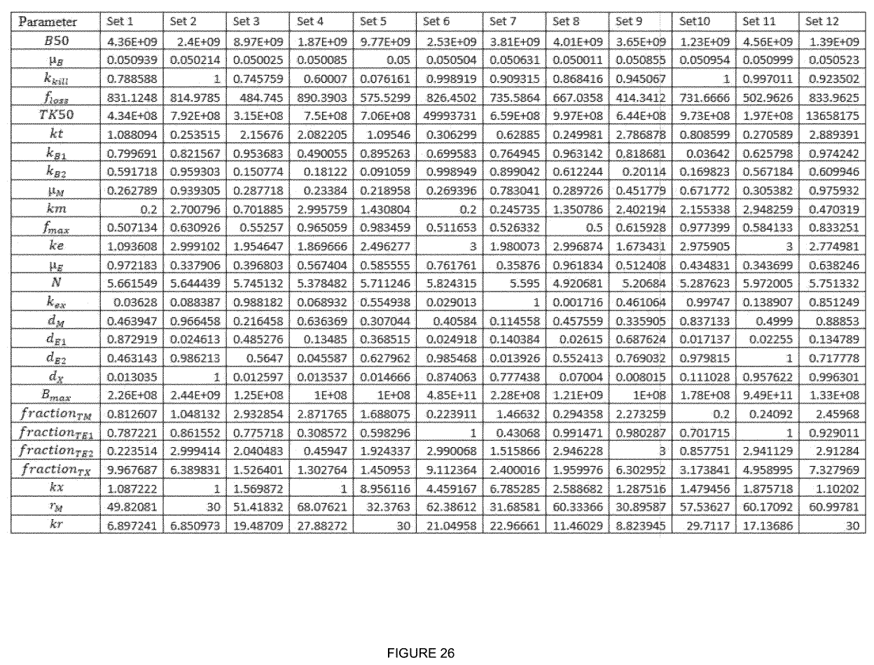

FIG. 26 shows 12 PR (partial responder) example parameter sets for the parameters of FIGS. 24A-24B, according to an embodiment.

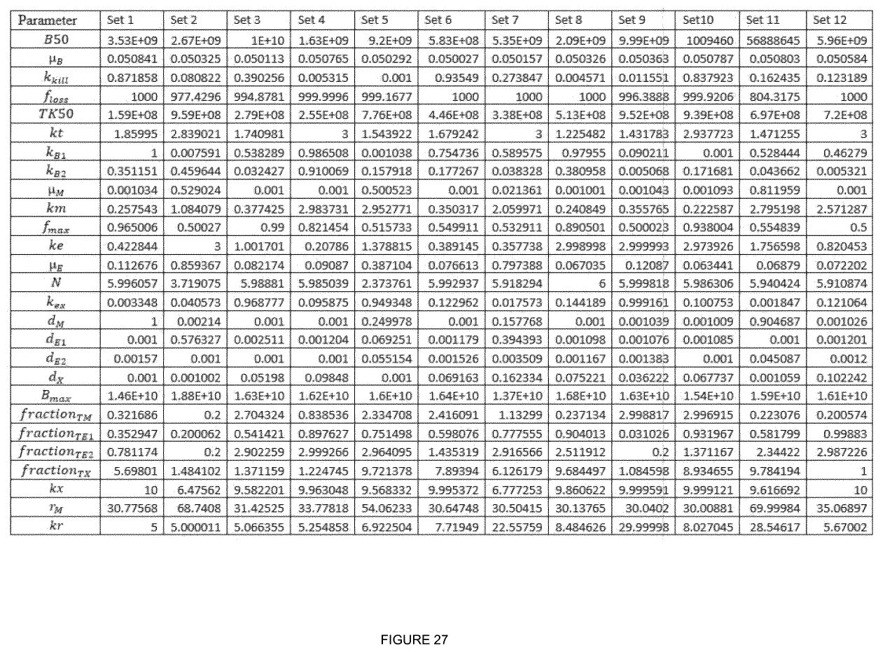

FIG. 27 shows 12 NR (non responder) example parameter sets for the parameters of FIGS. 24A-24B, according to an embodiment.

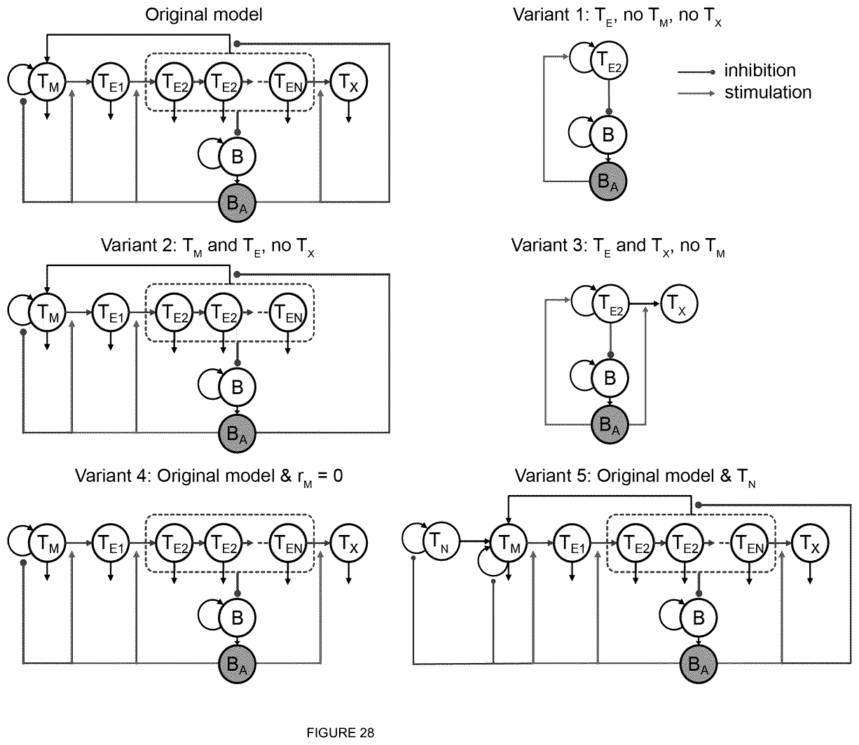

FIG. 28 shows a depiction of the structures of original model and potential variants, according to an embodiment.

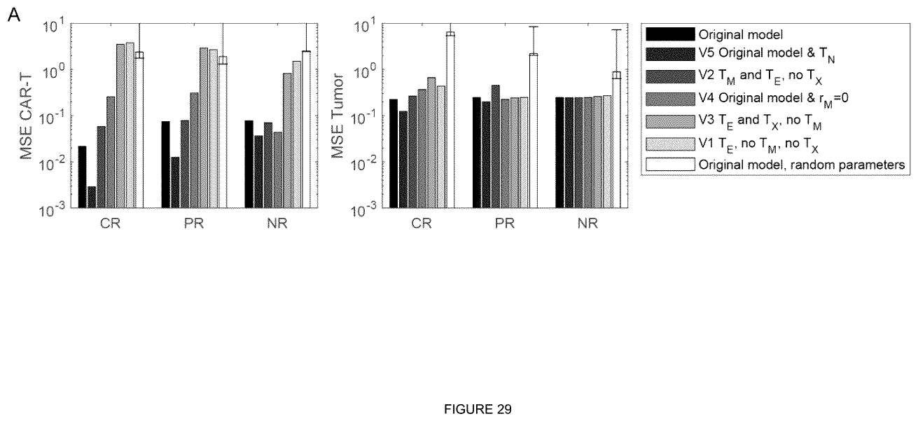

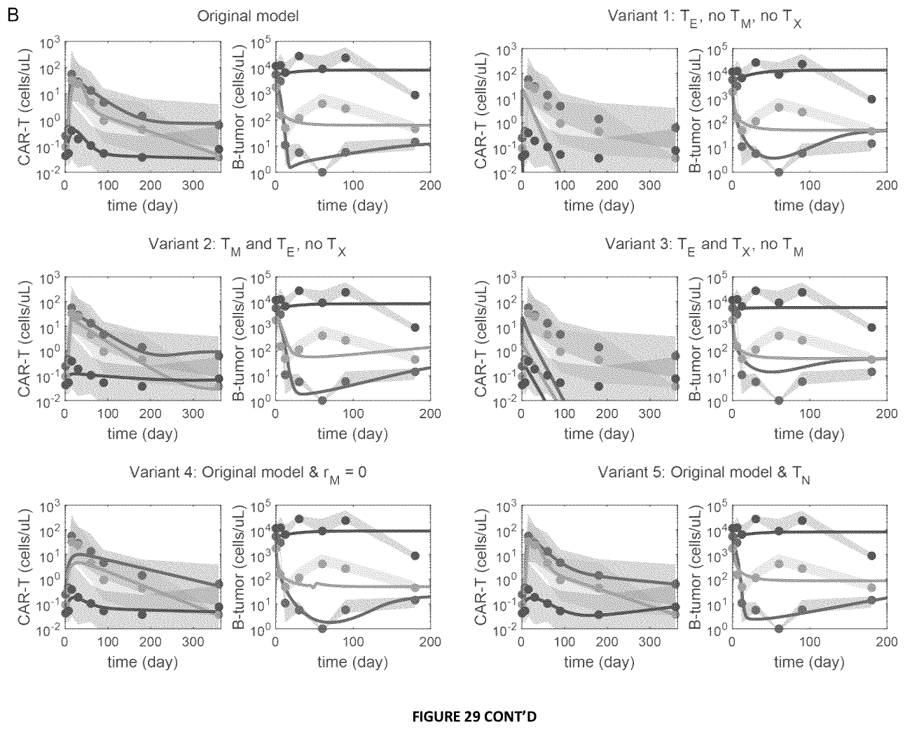

FIG. 29 shows fitting accuracy of a model against five structural variants. A Mean squared error (MSE) of the original (full) model and the five model variants fit the training data (from Fraietta), as well as random sampling of parameter search space for the original model. MSE plots are separated by fit to the pharmacokinetic and tumor dynamics, and rank ordered by overall goodness of fit. B Model simulations overlaid with training data for the original (full) model and five variants.

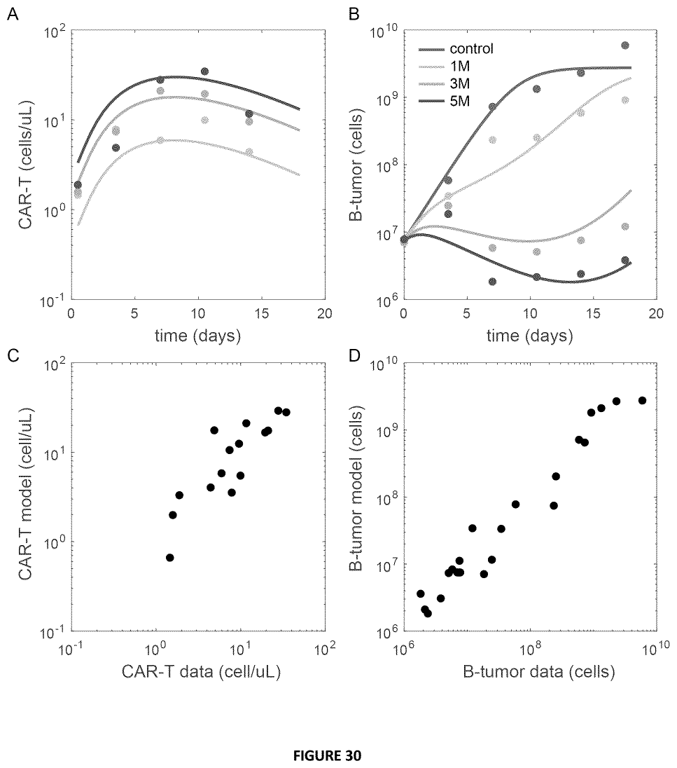

FIG. 30 shows model fitting to pre-clinical CD19-CAR-T data, according to an embodiment. NALM-6 xenograft bearing mice (injected with 106 tumor cells at day −6) were treated with increasing doses of Kymriah®, and tumor size measured by fluorescence imaging (e.g., reporter enzyme fluorescence (REF) imaging). For simplicity a 1:1 scaling relationship between photons/s and tumor cell number can be assumed. For fitting mouse as compared to clinical data, bounds on the tumor-related parameters Bmax (maximum tumor size) and TK50 (T cell EC50 driving tumor cell killing) was scaled down and the tumor growth rate (uB) was allowed to float between 0.1 and 1 per day. (A) Pharmacokinetic and (B) tumor dynamic data and model simulations for CAR-T doses of 0, 1, 3 and 5 million cells. Goodness of fit plots for the CAR-T pharmacokinetic (C) and tumor dynamics (D), with Pearson correlation coefficients of 0.88 and 0.84 respectively.

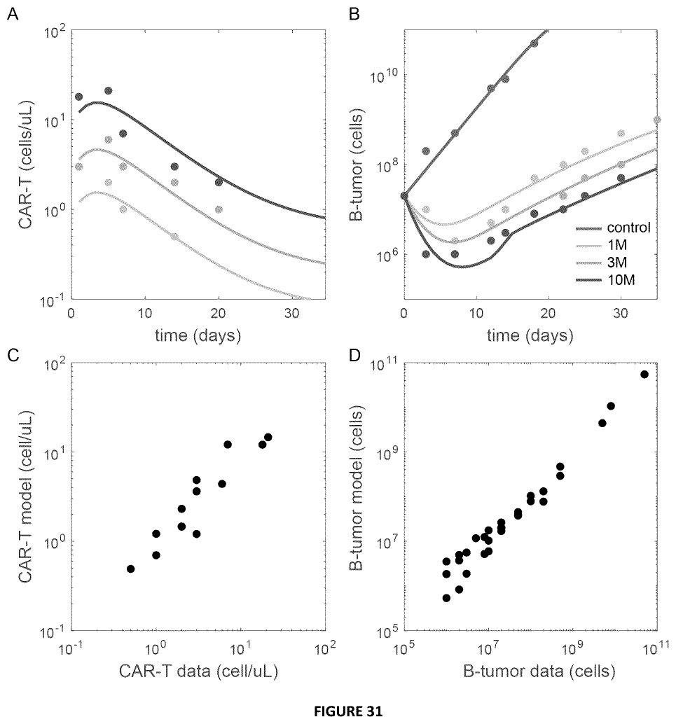

FIG. 31 shows model fitting to pre-clinical BCMA-CAR-T data. according to an embodiment. MM1.s xenograft bearing mice (injected with 5×10⁶ tumor cells at day −14 to −8) were treated with increasing doses of the research-grade CAR-T ‘BCMA-R2’. and tumor size measured by fluorescence imaging (e.g., reporter enzyme fluorescence (REF) imaging). For simplicity a 1:1 scaling relationship between photons/s and tumor cell number was assumed. For fitting mouse as compared to clinical data. bounds on the tumor-related parameters Bmax (maximum tumor size) and TK50 (T cell EC50 driving tumor cell killing) were scaled down and the tumor growth rate (uB) was allowed to float between 0.1 and 1 per day. (A) Pharmacokinetic and (B) tumor dynamic data and model simulations for CAR-T doses of 0, 1, 3 and 10 millions cells. Goodness of fit plots for the CAR-T pharmacokinetic (C) and tumor dynamics (D), with Pearson correlation coefficients of 0.92 and 0.99respectively.

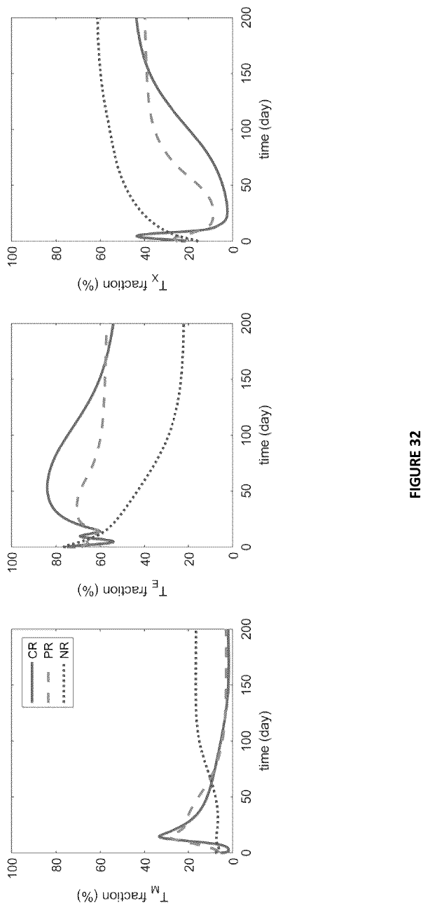

FIG. 32 shows simulated frequencies of memory (T_{M}), effector (T_{E}). and exhausted (Tₓ) CAR-T cells for CR, PR, and NR patient groups shown in FIG. 1.

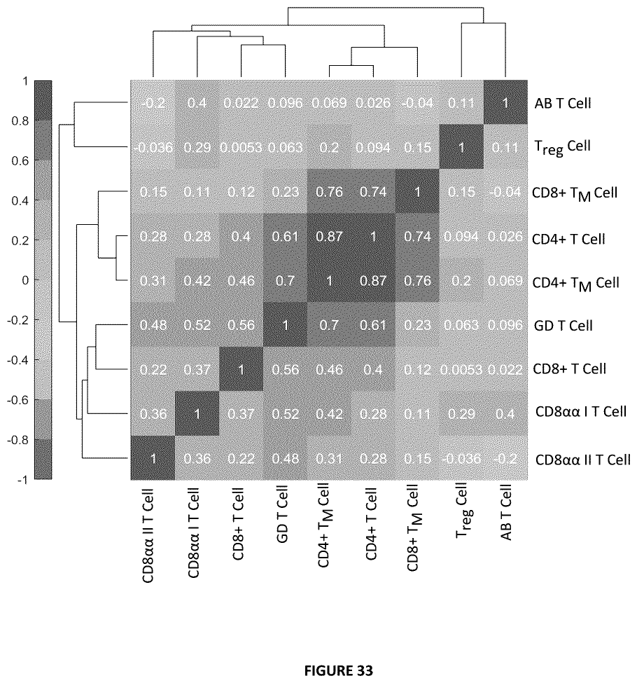

FIG. 33 shows pairwise Pearson correlation coefficients between Thymic Atlas cell population gene signatures computed using ssGSEA scores from Fraietta et al. RNAseq data. Note signatures for CD8+ T_{M}, CD4+ T_{M}, and CD4+ T cells are tightly correlated.

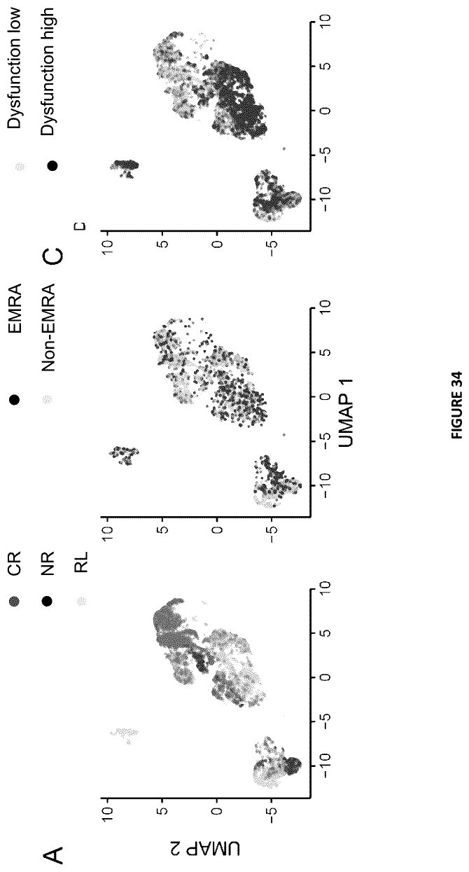

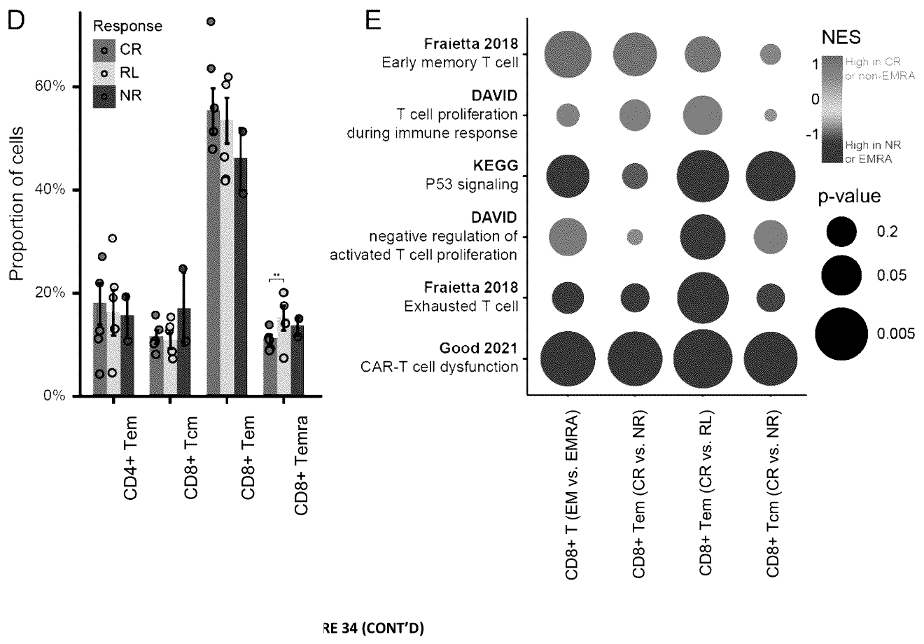

FIG. 34 shows single cell RNA sequencing of twelve pre-infusion CAR-T products classified by response. A UMAP projection of cells annotated by response category. CR: complete response. RL: relapsed. NR: non-response. B UMAP projection of cells annotated as TEMRA using SingleR58 with a reference dataset of healthy. donor-derived T cells (Bai et al., 2021). C Cell type frequencies for the CR, RL, and NR groups. Only cell types for which there is at least 5% representation in the sample are shown. Data is shown as mean+−standard error, with individual sample frequencies overlaid as dots. Significance is shown for all significant comparisons between CR and NR according to a Wilcoxon rank-sum test (p<=0.05). D Per-cell type GSEA for selected pathways which show significant enrichment (p<=0.05) in at least one cell type. A positive normalized enrichment score (NES) indicates higher enrichment in CR/non-exhausted cells.

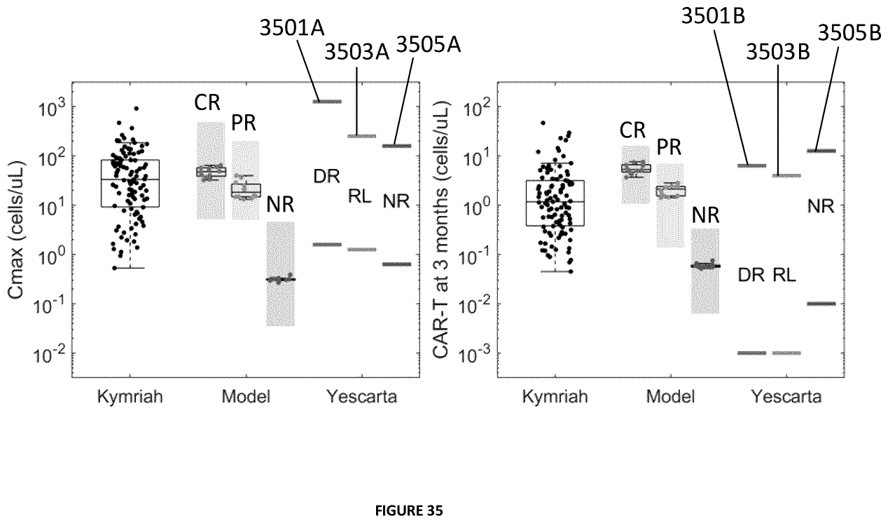

FIG. 35 shows comparative pharmacokinetics of Kymriah® in B-ALL. the model described herein of Kymriah® in CLL, and Yescarta® in LCBCL. Cmax and C(t=3 month) distributions for Kymriah® in B-ALL and CLL were obtained via simulations of the Stein et al. model (see. e.g., Stein, A. M. et al. Tisagenlecleucel Model-Based Cellular Kinetic Analysis of Chimeric Antigen Receptor-T Cells. Cpt Pharmacometrics Syst Pharmacol 8, 285-295(2019), the contents of which are incorporated by reference herein in its entirety) and the model described herein, respectively. Data for Yescarta® was digitized from Locke et al. (see, e.g., Locke, F. L. et al. Tumor burden, inflammation, and product attributes determine outcomes of axicabtagene ciloleucel in large B-cell lymphoma. Blood Adv 4. 4898-4911 (2020), the contents of which are incorporated by reference herein in its entirety). The distributions shown for Kymriah® and the model described herein are as in FIG. 5C for the left panel (Cmax). The group of boxplots labelled Model show the Cmax or C (t=3 month) for each of the three populations (CR, PR, and NR) with the background of the range of Cmax or C (t=3 month) obtained from the clinical PK data. Bar pairs 3501A, 3501B, 3503A, 3505B, 3505A, 3505B for Yescarta® show hand-estimated ranges of Cmax and C (t=3 month) while the labels (DR, durable response; RL: relapse; and NR: no response) are plotted at the hand-estimated medians.

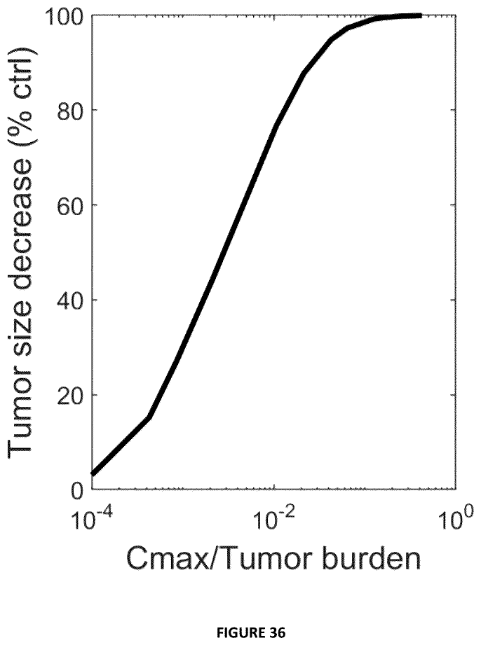

FIG. 36 shows Cmax/Tumor burden vs. tumor response simulations for Abecma®. The model fit to Abecma® phase1 data was simulated at CAR-T doses ranging from 0.1 to 1000 million cells. Tumor shrinkage, compared to untreated control, was calculated at day 60. The response covariate follows the same trend as that observed for Yescarta® in diffuse large B-cell lymphoma (DLBCL) and predicted for Kymriah®.

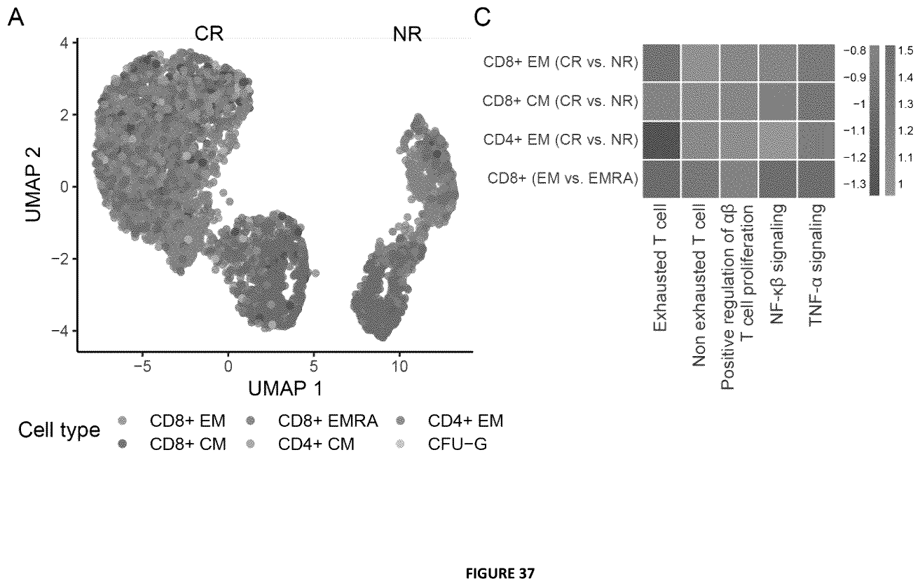

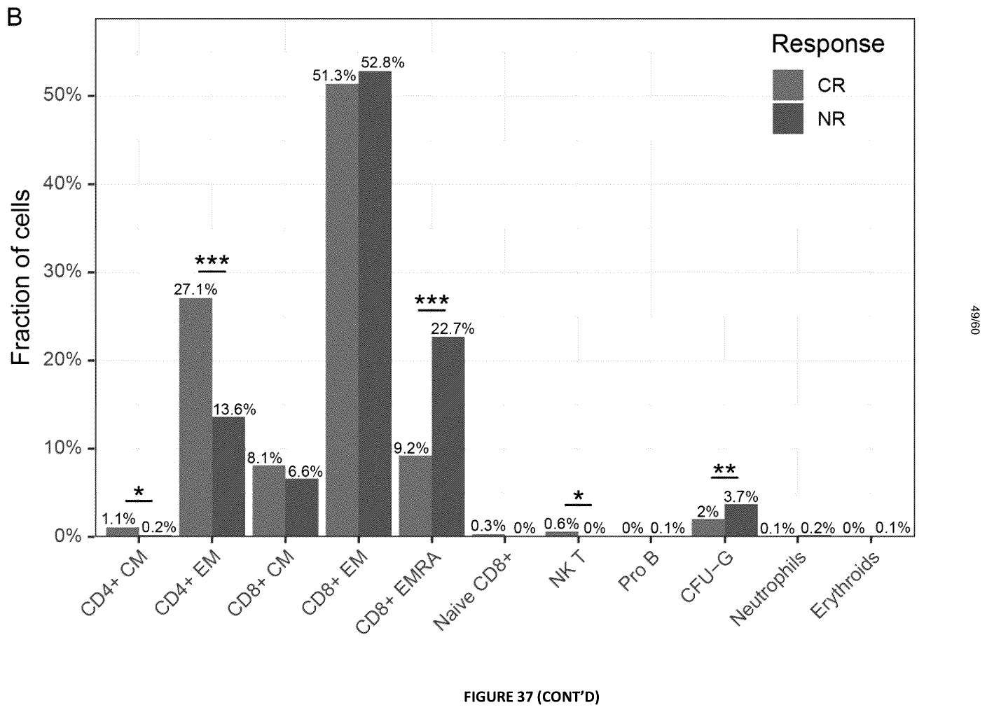

FIG. 37 shows single cell RNA sequencing of two pre-infusion CAR-T products separate by response, according to an embodiment. A Single cells were labeled using SingleR and form distinct clusters in UMAP space. B Cell type proportions in the CAR-T product separated by patient response status reveal differential frequencies of several canonical cell types. C Gene set enrichment analysis within memory T cell subtypes (for cell types with more than 1% of cells annotated as such per group). Cells are labelled according to their normalized enrichment statistic: CR indicates high in CR and NR indicates high in NR (with the exception of the final row, which indicates high in the CD8+ EMRA subtype compared to other CD8+ subtypes). EM: effector memory; CM: central memory; EMRA: effector memory re-expressing CD45RA; CFU-G: colony-forming units of granulocytes.

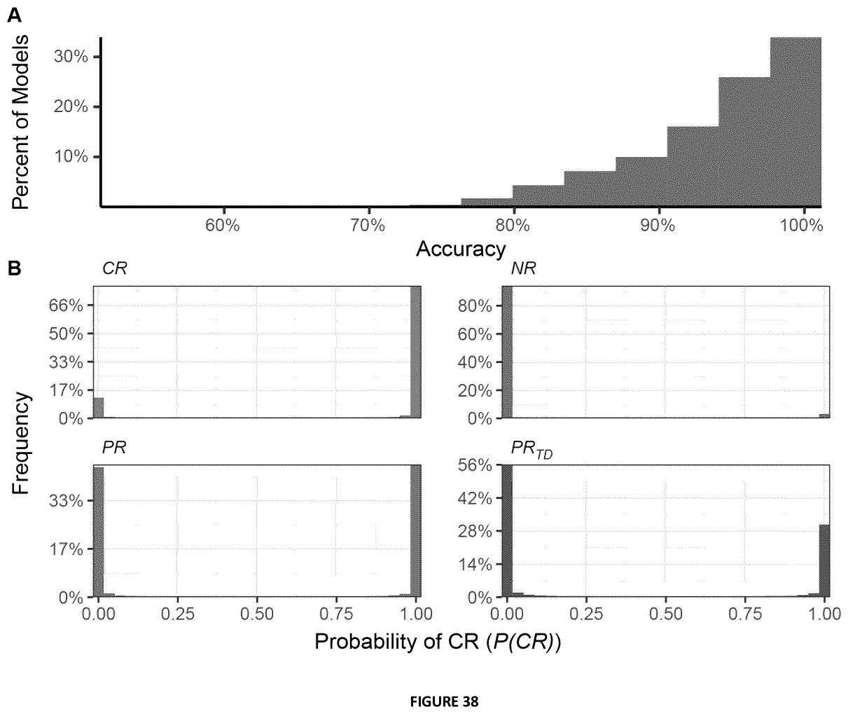

FIG. 38 shows that Kymriah® response in CLL can be accurately predicted using a ssGSEA-based classifier, according to an embodiment. A A stochastic workflow was used to parameterize a family of 12,000 multivariate logistic regression classifiers using sample ssGSEA scores as input features. Leave-one-out cross validated (LOOCV) accuracy distribution of the 12,000 parameterized classifiers, using 0.5 as a threshold. B Individual predictions separated by response category. CR and NR samples are accurately classified. while PR and PR_{ID} samples (not used in model training) classify as mid-way between CR vs. NR groups.

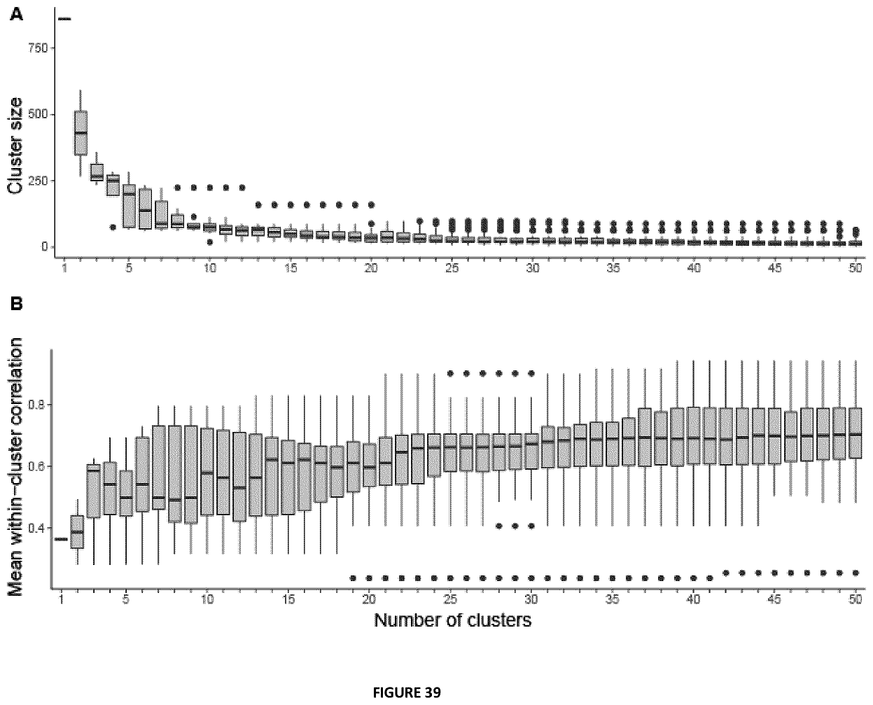

FIG. 39 shows (A) Cluster size and (B) mean within-cluster correlation distribution based on a variable number of separate clusters calculated for the 864 gene signatures differentially enriched between CR and NR groups.

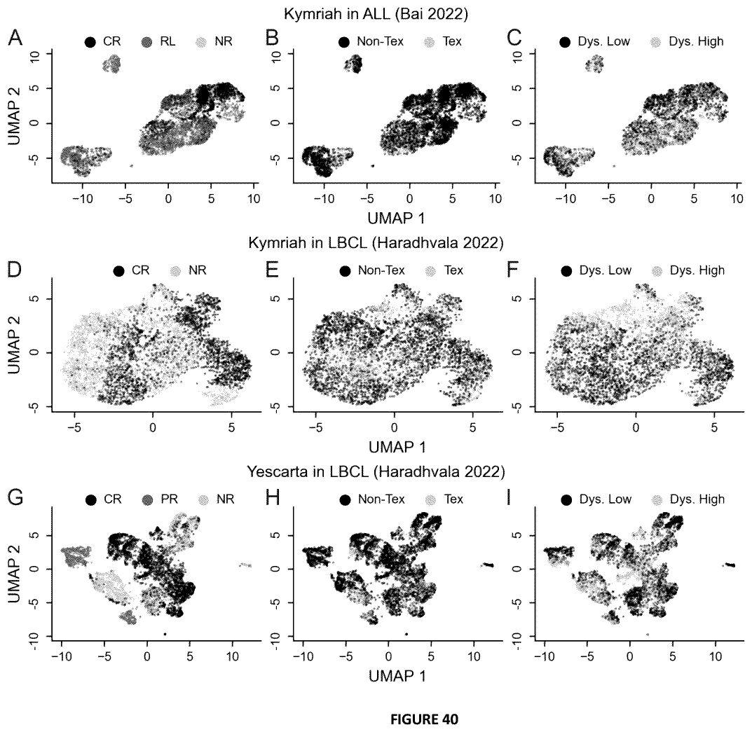

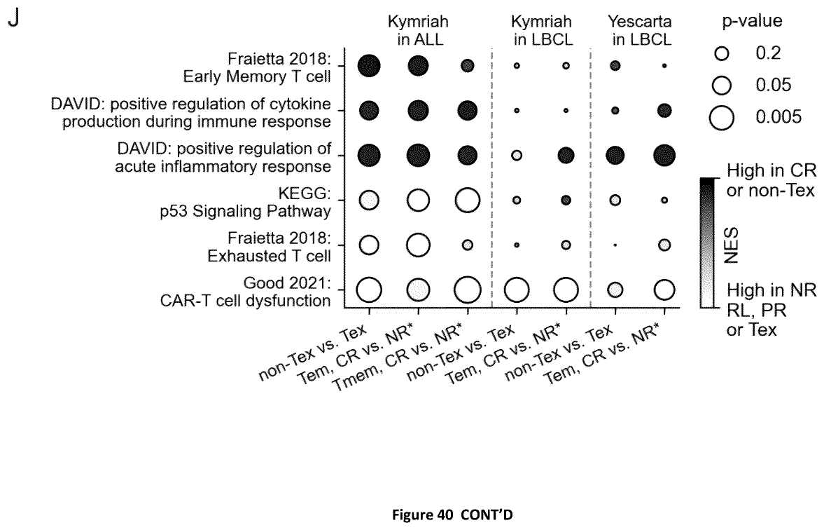

FIG. 40 shows that single cell RNA sequencing of pre-infusion CAR-T products reveals cell-intrinsic defects associated with non-durable response. according to an embodiment. Uniform manifold approximation and projection (UMAP) projections of three datasets representing Kymriah® in acute lymphoblastic leukemia (ALL) (A-C). Kymriah® in large B-cell lymphoma (LBCL) (D-F), and Yescarta® in LBCL (G-I). A,D,G UMAP projections annotated by response category. CR; complete response, RL; relapsed, PR: partial response, NR; non-response. B,C,H UMAP projections annotated as exhausted using ProjectTILs (“Tex”, exhausted T cells, “Non-Tex”, non-exhausted T cells). C,F,I UMAP projections annotated for high (above mean) (labelled “Dys. High”) or low (below mean) (labelled “Dys. Low”) CAR-T cell dysfunction signature from Good et al. E Per-cell type GSEA for select pathways, comparing both exhausted vs. non-exhausted, and CR vs. PR/RL/NR categories within cells annotated as T effector-memory (Tem via ProjecTILs) or early memory (Tmem: CD8+CD45RO-CD27+ via CITESeq). In some implementations, CITESeq refers to a sequencing-based method that simultaneously quantifies cell surface protein and transcriptomic data, e.g., mRNA, within a single cell readout. A positive normalized enrichment score (NES) indicates higher enrichment in CR/non-exhausted cells. *NR=NR/RL or NR/PR.

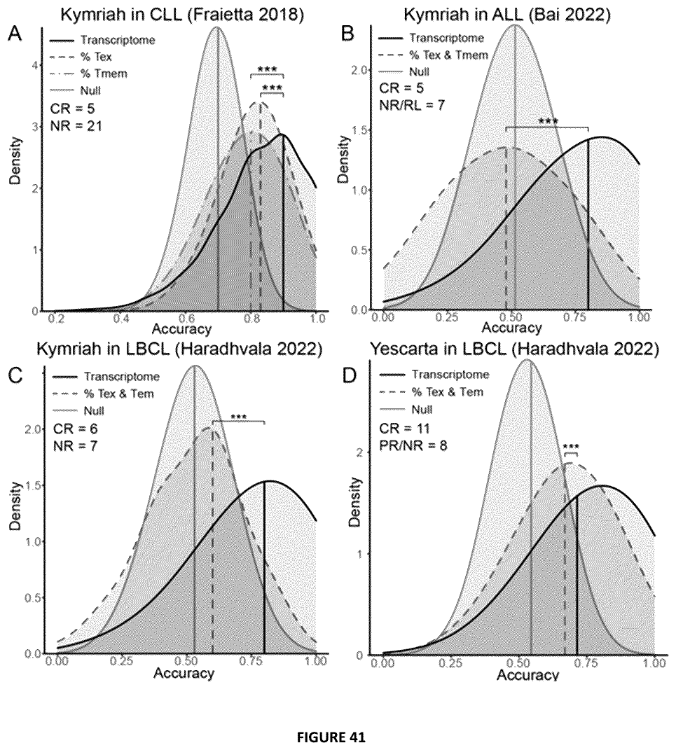

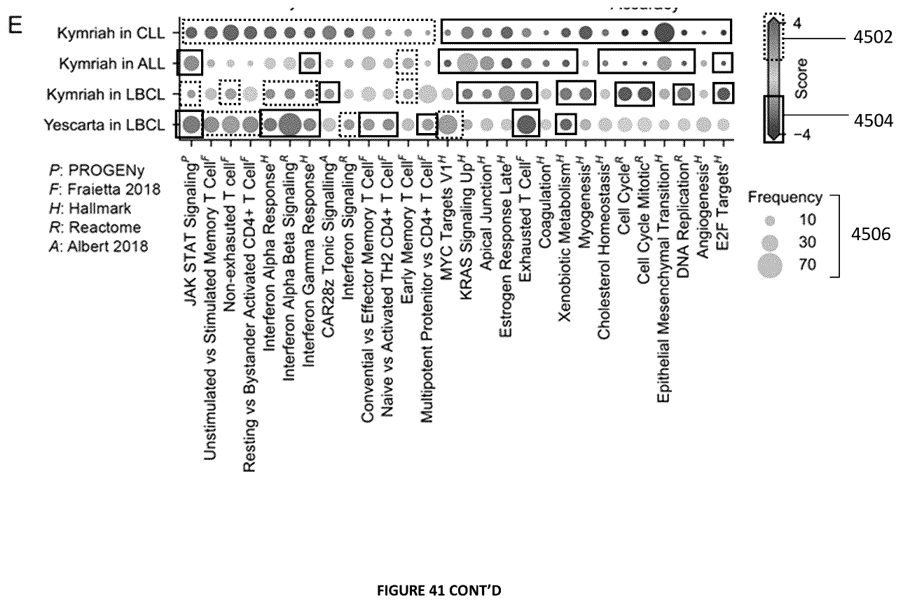

FIG. 41 shows that CD19-CART response can be predicted from infusion products using a ssGSEA-based transcriptome classifier with better accuracy than T cell immunophenotypes, according to an embodiment. Distribution of predictive accuracies are shown for 2500 iterations using 60:40 train: test split cross validation. Results from the transcriptome-based ssGSEA classifier are compared to (A) classifiers based on reported T memory (CD8+CD45RO-CD27+) and T exhausted (CD8+PD1+) cell frequencies from Fraietta et al. (B) a bi-variate classifier based on calculated T memory (CD8+CD45RO-CD27+) and T exhausted (CD8+PD1+) cell frequencies from Bai et al. 2022 (C,D) bivariate classifiers based on T effector-memory and exhausted cell frequencies from ProjecTILs annotations of Haradhvala et al. (see, e.g., Haradhvala, N. J. et al. Distinct cellular dynamics associated with response to CAR-T therapy for refractory B cell lymphoma. Nat Med 1-12(2022). the contents of which are incorporated by reference herein in its entirety). Accuracy distribution resulting from null models (random classification) are shown as controls. *** indicates p<10-15. Wilcoxon rank-sum test. E CART response scorecard. representing the 28 gene signatures provided to the transcriptome classifier. ordered by differential GSEA in Fraietta et al. Bubble size indicates frequency of inclusion in the 2,500 trained models after feature selection, shade indicates differential enrichment between response groups by dataset. based on pseudo-bulked GSEA (Score=−1×sign (NES)×log10-Pval). Reference 4506 shows three examples of bubble sizes and the frequencies that they represent; the bigger the circle, the larger the frequency. More specifically, reference 4506 shows the bubble size that represents a frequency of 10, the bubble size that represents a frequency of 30, and the bubble size that represents a frequency of 70. Scores with shading within score range 4502 are NR/PR/RL-enriched. and scores with shading within score range 4504 are CR-enriched: in other words, circles within solid lined box=CR-enriched, and circles within dashed lined box, =NR/PR/RL-enriched. Gene signatures are annotated by source.

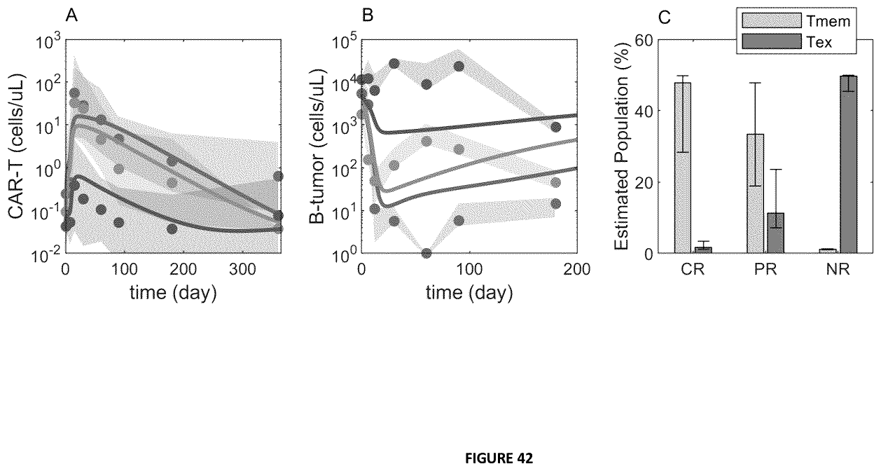

FIG. 42 shows model fitting results based on the hypothesis that the only distinguishing feature between CR, PR, and NR populations is the fraction of memory T and exhausted T cells in the CAR-T infusion product. Memory cell fraction (f Tm) and exhausted cell fraction (f_Tx) can be estimated as between 1-50% independently for the CR, PR, and NR populations while other model parameters can be estimated simultaneously using a single vector for the CR, PR, and NR populations. Simulations of best fit model (e.g., estimated by MSE minimization) from 12 optimization runs for (A) CAR-T pharmacokinetics and (B) tumor dynamics. (C) Estimated fraction of memory and exhausted cells (f_Tm, f_Tx) in CAR-T infusion products for CR, PR, and NR populations. Bars represent medians±25 percentile intervals from n=12 model fits.

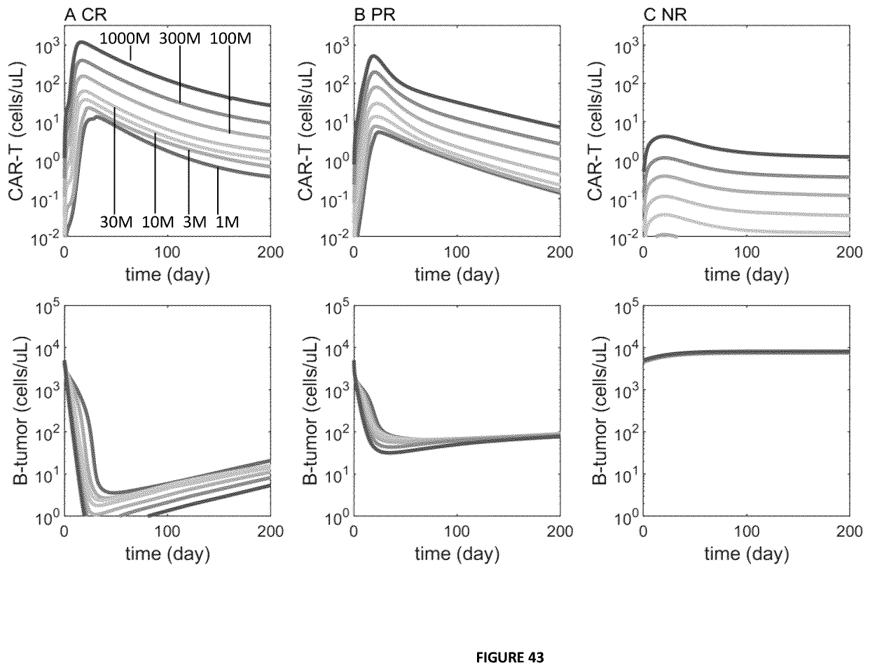

FIG. 43 shows simulated pharmacokinetic and tumor dynamic responses to increasing cell doses of pure memory cell populations from CR, PR, and NR population models. Simulations were run at doses of 1 (labelled “1M”). 3 (labelled “3M”), 10 (labelled “10M”), 30 (labelled “30M”), 100 (labelled “100M”), 300 (labelled “300M”) and 1000(labelled “1000M”) million cells using parameter sets estimated for CR (A), PR (B), and NR (C) populations. For direct comparison, the memory cell fraction was set to 100% for each.

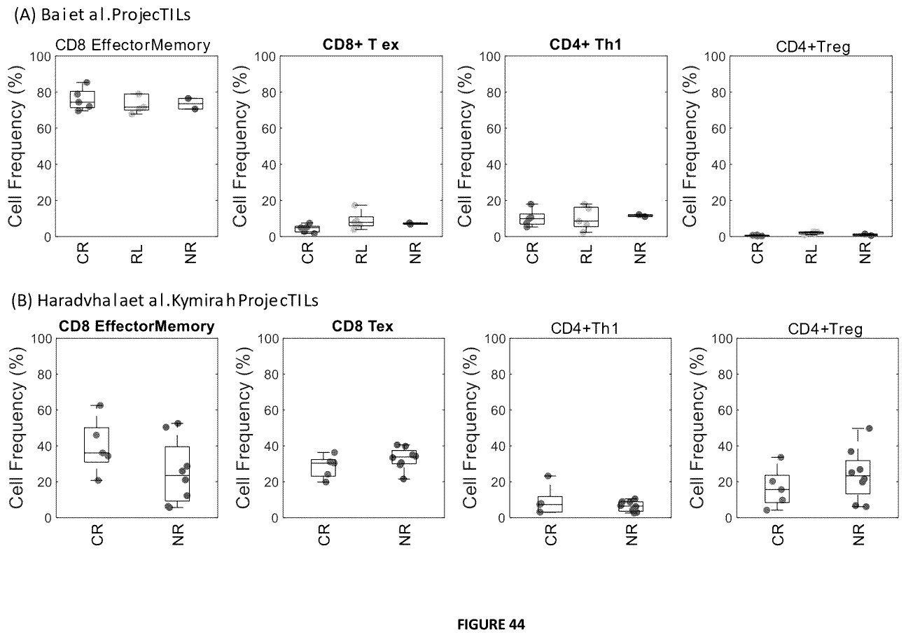

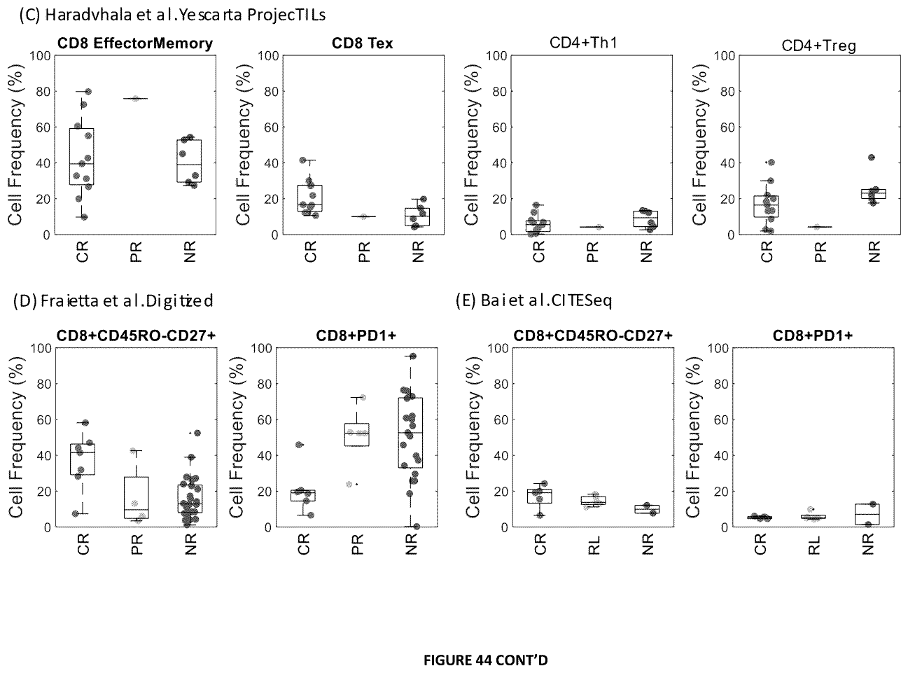

FIG. 44 shows T cell phenotyping of CAR-T infusion products. ProjecTILs-annotated cell frequencies by response category for (A) Kymriah® in ALL, digitized from Bai et al. 2022, n=12: (B) Kyrmiah® in LBCL, digitized from Haradhvala et al., n=11: (C) Yescarta® in LBCL, digitized from Haradhvala et al., n=19. Cell types annotated at frequencies of less than 5% are excluded; CD4+ Naïve, CD8+Naïve, CD8+ Tprecursor-exhausted (Tpex) and CD4+ follicular-helper (Tfh). D Immunophenotype-defined T early memory (CD8+CD45RO-CD27+) and exhausted (CD8+PD1+) cell frequencies by response category for Kymriah® in CLL, digitized from Fraietta et al., n=38. B Immunophenotype-defined Early memory and exhausted cell frequencies by response category in for Kymriah® in ALL, calculated from Bai et al. 2022. CITEseq antibody tags, n=12. Boxplots represent median±25 percentiles, and whiskers the min/max value or an additional 1.5-fold quartile distance.

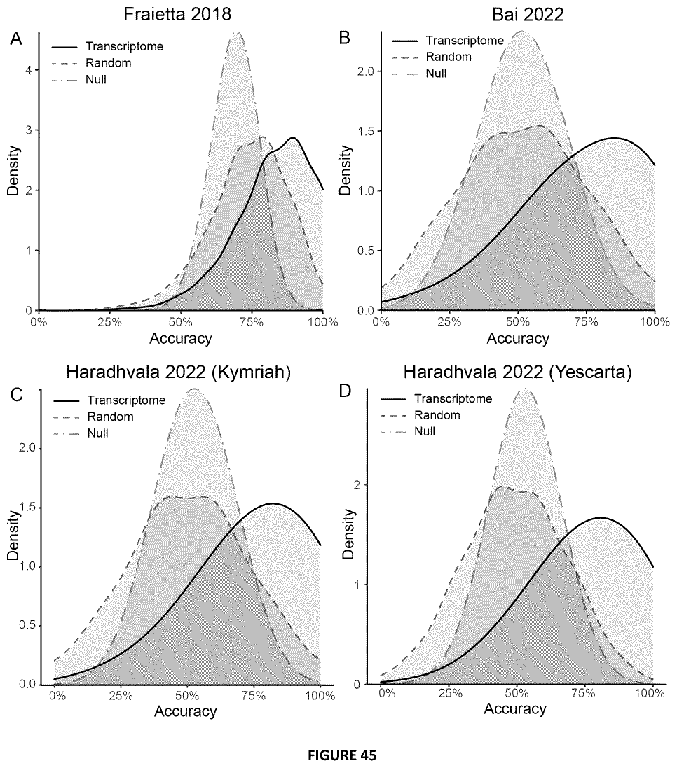

FIG. 45 shows transcriptome classifier performance as compared to null and random pathway models. Distribution of predictive accuracies are shown for 2500 iterations using 60:40 train:test split cross validation. Results from the 28-signature transcriptome-based ssGSEA classifier (“Transcriptome”) are compared null models (random classification; “null”) and an ssGSEA classifier trained on a randomized selection of pathways from the compendium (“Random”).

### EXAMPLE 1: Methods

### Clinical Data: Kymriah®(tisagenlecleucel, CTL019)

Mean pharmacokinetic and tumor dynamic profiles were digitized from a clinical study of Chronic Lymphocytic leukemia (CLL) patients treated with Kymriah® (tisagenlecleucel, CTL019), separated into Complete Responders (CR, n=8), Partial Responders (PR, n=5) and Non-responders (NR, n=25). Samples annotated as PRTD (late relapse into B-cell lymphoma) were excluded. Patients were treated with CAR-T doses ranging from 0.14 to 11×10⁸ cells. For parameter estimation a fixed dose of 108 cells was assumed. consistent with median dose used in this study and other clinical trials of Kymriah®. Tumor size data was reported as B cells/μL and was hence used directly in model fitting (assuming an initial tumor burden of 10¹⁰ total cells). Pharmacokinetics were reported as CD19-CAR transgene copies in peripheral blood (copies/μg genomic DNA) and were converted to cell numbers for mechanistic modelling (see below).

The non-linear mixed effects model of Kymriah® cellular kinetics was used to simulate population pharmacokinetics in refractory B cell acute Lymphoblastic leukemia (B-ALL). The model was parameterized using data compiled from two clinical studies, treated with a median dose of 108 cells (n=91). In some instances, pharmacokinetic profiles of Kymriah® in CLL patients do not to differ substantially from B-ALL patients. To compute distributions of exposure (AUC and Cmax), PK profiles for 1000 virtual patients were simulated. At each time step (0.1 days for 1 year). 1-99 percentiles were computed. and AUC and Cmax calculated from these percentiles.

### Clinical Data: Abecma® (idecabtagene vicleucel, BB2121)

Mean pharmacokinetic and tumor dynamic profiles were digitized from a Phase 1 dose escalation study (Locke et al.) of refractory Multiple Myeloma (MM) patients treated with Abecma R (n=33). separated by dose group (50, 150, 450 and 800×10⁶ cells). Tumor size data was reported as % change in serum BCMA levels. For model fitting initial tumor burden was assumed as 10¹⁰ cells and linear scaling between tumor burden and reported soluble BCMA. PK data was reported as transgene copies/μg DNA, and the same scaling factor as above was applied to convert to CAR-T cell counts. Mean PK and tumor dynamic profiles+/−standard deviation were digitized from a Phase 2 study in the same patient population (N=128). treated with 150 and 450×10⁶ cell doses (data not separated by dose) (see. e.g., Munshi. N. C. et al. Idecabtagene Vicleucel in Relapsed and Refractory Multiple Myeloma. New Engl J Med 384. 705-716 (2021), the contents of which are incorporated by reference herein in its entirety). Tumor dynamic data in this study were reported as serum BCMA (ng/ml). Data was converted to % change from baseline, again assuming initial tumor burden of 10¹⁰ cells for comparison to model simulations.

### Scaling Factors and Virtual Population

To estimate a scaling factor between transgene counts and cell numbers, data from Kalos et al. (see, e.g., Kalos, M. et al. T Cells with Chimeric Antigen Receptors Have, Potent Antitumor Effects and Can Establish Memory in Patients with Advanced Leukemia. Sci Transl Med 3. 95ra73-95ra73 (2011), the contents of which are incorporated by reference herein in its entirety) was used wherein both counts/ug and total circulating CD19+ cells were reported. estimated as ˜10⁴. For conversions between total cell numbers and cells/μL for plotting a total blood volume of 2 μL in humans and 2 μL in mice was assumed.

### Model Structure and Assumptions

Three functionally distinct T cell populations can be encoded: T memory cells (T_{M}), capable of long-term regenerative capacity (self-renewal) and differentiation: T effector cells (T_{E}) which arise from memory population and are responsible for direct killing of tumor cells: and T exhausted cells (Tₓ) that lack effector function and proliferative capacity. T effectors can expand through N population doublings but can lack the capacity for self-renewal. One aspect of the mechanism-based description of T cell differentiation control is a toggle-switch sensor of tumor antigen, encoded as a Hill equation. This toggle-switch regulates: the rate of T memory cell self-renewal vs. differentiation; proliferation rate of T effectors; exhaustion rate of T effectors; and regeneration of T memory cells from T effectors.

This control of T-cell fates can be described via a system of nonlinear ordinary differential equations (ODEs), for example:

$$\frac{d {T}_{M}}{d t} = 2 \cdot {μ}_{M} \cdot {f}_{max} \cdot ( 1 - \frac{{B}_{A}^{k m}}{B 5 {0}^{k m} + {B}_{A}^{k m}} ) \cdot {T}_{M} + {r}_{M} \cdot ( 1 - \frac{{B}_{A}^{k r}}{B 5 {0}^{k r} + {B}_{A}^{k r}} ) \cdot {T}_{E 2} - {d}_{M} \cdot {T}_{M} ,$$

$$\frac{d {T}_{E 1}}{d t} = 2 \cdot {μ}_{M} \cdot ( 1 - {f}_{max} \cdot ( 1 - \frac{{B}_{A}^{k m}}{B 5 {0}^{k m} + {B}_{A}^{k m}} ) ) \cdot {T}_{M} - {μ}_{E} \cdot ( \frac{{B}_{A}^{k e}}{B 5 {0}^{k e} + {B}_{A}^{k e}} ) \cdot {T}_{E 1} - {d}_{E 1} \cdot {T}_{E 1}$$

$$\frac{d {T}_{E 2}}{d t} = {μ}_{E} \cdot {2}^{N} ( \frac{{B}_{A}^{k m}}{B 5 {0}^{k m} + {B}_{A}^{k m}} ) \cdot {T}_{E 1} - {k}_{e x} ( \frac{{B}_{A}^{k x}}{B 5 {0}^{k x} + {B}_{A}^{k x}} ) \cdot {T}_{E 2} - {d}_{E 2} \cdot {T}_{E 2} ,$$

$$\frac{d {T}_{X}}{d t} = {k}_{e x} ( \frac{{B}_{A}^{k x}}{B 5 {0}^{k x} + {B}_{A}^{k x}} ) \cdot {T}_{E 2} - {d}_{X} \cdot {T}_{X} .$$

Here, the self-renewal and differentiation of memory cells occurs at rate um and is regulated through Hill equation switches that depend on the B cell antigen B_{A}. The parameter fₘₐₓ describes the fraction of memory cells that self-renew versus differentiate to become effector cells. Memory cells are regenerated (with rate parameter T_{M}) from the T_{E2} population. The effector populations were divided into two subgroups: T_{E1} and T_{E2} that describe the non-tumor killing and tumor killing effector populations, respectively. This division can be for mathematical simplicity: the non-tumor killing subgroup differentiates from the memory cells and forms the initial pool of effector cells that further differentiates (with rate parameter μ_{g}) to cytotoxic effector cells (T_{E2}). For parameter estimation routines N population doublings can be encoded in a single source term in the T_{E2} equation instead of using a hierarchy of ODEs, each tracking the number of cells that have undergone n divisions. T effector cells become exhausted with rate parameter kₑₓ, and T cell populations are removed with corresponding rate parameters d_{M}, d_{E1}, d_{E2}, dₓ. Note that the toggle switch, encoded as a Hill function in B cell antigen BA, has the same half-maximum parameter B50 across all T cell populations, but different exponents (km, kr, km, ke, and kx) to account for presumed differential dose-response relationships.

The dynamics of B cell tumors can be modeled with logistic growth with rate μ_{B} and carrying capacity Bₘₐₓ, and non-linear tumor killing through effectors with rate kₖᵢₗₗ as well as the production and decay of B cell antigen B_{A}, for example:

$$\frac{d B}{d t} = {μ}_{B} \cdot ( 1 - \frac{B}{{B}_{max}} ) \cdot B - {k}_{k i l l} \cdot ( \frac{{T}_{E 2}^{k t}}{T K 5 {0}^{k t} + {T}_{E 2}^{k t}} ) \cdot B ,$$

$$\frac{d {B}_{A}}{d t} = {k}_{B 1} \cdot B - {k}_{B 2} \cdot {B}_{A} .$$

By encoding proliferation/differentiation as a driven by tumor antigen (B_{A}) rather than tumor cell number (B), the production degradation rates (k_{B1}, k_{B2}) creates a surrogate transient compartment. This allows for a time delay between changes in tumor burden and responsiveness of T cell fates. Transient compartments can be employed in PK/PD modelling to connect drug concentration to measured pharmacodynamic response.

To map cell dosing to initial condition, two empirical, rapid reactions can be implemented. First, a proportion of the infused cell dose is rapidly lost to account for discrepancy between cell dose and the initial conditions observed both clinically and in pre-clinical models when cells/μL are reported. Second, the initial cell dose rapidly converts into the four T cell subpopulations such that the composition does not need to be pre-specified and can be determined through parameter estimation. This reaction accounts for the fact that CAR-T products are comprised of mixed populations of T cells (memory, effector and exhausted states), this composition may vary and may not be specified in clinical data. Rather than pre-specifying the composition via initial conditions, the rapid conversion reaction allows the fractions to be estimated as model parameters. This can be achieved via the following set of equations where Dose is the CAR-T Dose administered and DoseX is the remaining dose that is fractionated into the T cell subpopulations, for example:

$$\frac{d D o s e}{d t} = - ( 1 + {f}_{l o s s} ) \cdot Dose ,$$

$$\frac{d D o s e X}{d t} = Dose - ( {fraction}_{T M} + {fraction}_{T E 1} + {fraction}_{T E 2} + {fraction}_{TX} ) \cdot DoseX ,$$

$$\frac{d {T}_{M}}{d t} = {fraction}_{T M} \cdot DoseX ,$$

$$\frac{d {T}_{E 1}}{d t} = {fraction}_{T E 1} \cdot DoseX ,$$

$$\frac{d {T}_{E 2}}{d t} = {fraction}_{T E 2} \cdot DoseX ,$$

$$\frac{d {T}_{X}}{d t} = {fraction}_{T X} \cdot DoseX .$$

Zero-limits can be applied to cell populations to limit artificial regrowth. In some implementations, that is, if any cell population had a fractional number (<1), that cell population can be set to 0. Particle swarm optimization (PSO) can be used to estimate the model parameters based on minimization of the log-mean squared error between model simulations and data. The model structure can be encoded in Matlab® SimBiology® (R2021a) and particle swarm optimization (PSO) can be used to estimate the model parameters based on minimization of the log-mean squared error (MSE) between model simulations and data, using the particleswarm function with 100 particles×100 iterations, and the LLQ set at 10⁶ total CAR-T cells. The model can be fit separately to the CR, PR, and NR populations by applying the PSO algorithm 12 times for each population, generating a total of 36 parameter sets for analysis. See FIGS. 24A-24B for a list of model parameters, units, and lower and upper bounds used in the PSO algorithm. See FIG. 25 for the 12 CR parameter sets, FIG. 26 for the 12 PR parameter sets, FIG. 27 for the 12 NR parameter sets.

Model variants based on alternate T cell population structures were also assessed for the ability to fit the data, however none outperformed the above formulation (see below and FIG. 28, FIG. 29).

### Local Parameter Sensitivity Analysis

Local parameter sensitivity coefficients (LPSC) can be computed by simulating the model and computing the CAR-T AUC and tumor AUC in response to a 10% increase in estimated parameter values across the 36 parameter sets characterizing CR/PR/NR populations.

Coefficients can be calculated based on the median change in AUC for each population according to the formula:

$$L P S {C}_{Y \vert X} = \frac{Δ Y / Y}{Δ X / X} \cdot 100$$

wherein Y is the specified model output (CAR-T or tumor AUC) and X the specified parameter.

Virtual populations

Virtual populations can be created from the CR/PR/NR population fits by Monte Carlo sampling underlying parameter sets while varying CAR-T dose (10⁷-10⁹ cells) and initial tumor burden (8.5×10⁸-2.7×10₁₀ cells) within reported ranges by log-uniform sampling.

### Modelling Workflow

One strategy for model-based integration of the disparate datasets is to 1. Fit the PKPD model independently to the Fraietta et al. CR, PR, and NR profiles. 2. Create virtual populations from this model and compare the predicted population PK variance against Kymriah (R) data from Stein et al. and covariates of response against Yescarta® data from Locke et al. 3. Fit the PKPD model to Abecma® dose-response data from Raje et al. to understand mechanisms underlying the response covariates.

### RNASeq Analysis

Analysis of bulk RNASeq data was implemented within R version 4.1.1. Read count data was downloaded from supplement provided by Fraietta et al. (see, e.g., Fraietta, J. A. et al. Determinants of response and resistance to CD19 chimeric antigen receptor (CAR) T cell therapy of chronic lymphocytic leukemia. Nat Med 24, 563-571 (2018), the contents of which are incorporated by reference herein in its entirety), TMM normalized and converted to log (Counts per million) by applying Voom transformation. Differential gene expression was implemented with limma (see, e.g., Ritchie, M. E. et al. limma powers differential expression analyses for RNA-sequencing and microarray studies. Nucleic Acids Res 43, e47-e47 (2015), the contents of which are incorporated by reference herein in its entirety) and gene signature enrichment estimated with single sample GSEA (see, e.g., Barbie, D. A. et al. Systematic RNA interference reveals that oncogenic KRAS-driven cancers require TBK1. Nature 462, 108-112(2009), the contents of which are incorporated by reference herein in its entirety). Normalized ssGSEA scores for each pair of sample and gene signature can be calculated as, for example:

$$N i j = \frac{A i j - MIN ( A )}{MAX ( A ) - MIN ( A )}$$

Wherein A is the matrix of ssGSEA signature scores (i) x samples (j).

Gene signatures for cell signaling pathways were compiled from PROGENy (10 gene sets) (see, e.g., Schubert, M. et al. Perturbation-response genes reveal signaling footprints in cancer gene expression. Nat Commun 9, 20 (2018), the contents of which are incorporated by reference herein in its entirety), BioCarta (217 gene sets) (see, e.g., BioCarta Pathways. https://maayanlab.cloud/Harmonizome/dataset/Biocarta+Pathways, the contents of which are incorporated by reference herein in its entirety, Reactome (674 gene sets) (see e.g. Gillespie, M. eta al. The reactome pathway knowledgebasse 2022. Nucleic Acids Res 50, gkab1028-(2021), the contents of which are incorporated by reference herein in its entirety), Hallmark (50 gene sets) (see, e.g., Liberzon A, Birger C, Thorvaldsdóttir H, et al (2015) The Molecular Signatures Database Hallmark Gene Set Collection. Cell Syst 1:417-425.https://doi.org/10.1016/j.cels.2015.12.004, the contents of which are incorporated by reference herein in its entirety) and DAVID (6577 gene sets) (see, e.g., Huang, D. W., Sherman, B. T. & Lempicki, R. A. Systematic and integrative analysis of large gene lists using DAVID bioinformatics resources. Nat Protoc 4, 44-57 (2009), the contents of which are incorporated by reference herein in its entirety), cell population signatures from those published in Fraietta et al. (7 sets of cell population signatures) as well as a single-cell atlas of thymic development (13 sets of cell population gene signatures) (see, e.g., Park, J.-E. et al. A cell atlas of human thymic development defines T cell repertoire formation. Science 367, eaay3224 (2020), the contents of which are incorporated by reference herein in its entirety) and individual signatures for CAR-T dysfunction and CD28z tonic signaling (Tables 1, 2).

TABLE 1
Signatures for CAR-T Dysfunction

| | | | | | |
|---|---|---|---|---|---|
| HAVCR2 | CSF1 | SOLE | CCL3 | KLRC2 | IL2RA |
| RGS16 | TNFRSF9 | ID3 | CCL4 | CDK6 | AHI1 |
| LAYN | LYST | SOX4 | KLRC1 | PLS3 | RBPJ |
| SRGAP3 | TNFRSF18 | CD9 | KLRD1 | AFAP1L2 | GZMB |
| DUSP4 | NOFIP2 | PHLDA1 | KLRB1 | CTSW | GNLY |

TABLE 2
Signatures for CD28z Tonic Signaling

| | | | | | |
|---|---|---|---|---|---|
| TNFSF10 | CD95 | CD8A | CD69 | RUNX2 | CD127 |
| IFNG | PRF | BCL6 | TBET | CCR7 | IL18RAP |
| CD3E | CTLA4 | FOXO1 | CD154 | CMAF | AHR |
| CD27 | ICOS | JAK3 | RNX1 | TMEM2 | CCR6 |
| SRP14 | HMGB1 | IFNAR1 | CD247 | IFNA1 | FOSL |
| CD25 | HSAFX | STAT3 | CCR4 | HELIOS | |
| RANTES | TGFBR2 | RGS16 | CCR5 | AKURA | |

Additionally, two supplemental datasets are submitted herewith (supplemental datasets A, B). Supplemental dataset A shows the top 30 pathways (“model counts”), scored as to how many times they are included in the final models (both for the Fraietta and Bai versions). Supplemental dataset B shows the top 30 pathways (“model counts”), scored as to how many times they are included in the final models (for the Fraietta, Bai, Haradhvala (Yescarta®) and Haradhvala (Kymriah®) versions).

### ssGSEA-Based Response Classifier

Single sample GSEA scores corresponding to gene signatures that were differentially enriched between CR and NR groups (30, based on an FDR-adjusted p-value of 0.05) can be used to build a logistic regression-based classifier of response status:

$$log ( \frac{p ( C R )}{1 - p ( C R )} ) = {β}_{0} + {β}_{1} \cdot {ssGSEA}_{1} + {β}_{2} \cdot {ssGSEA}_{2} + … + {β}_{N} \cdot {ssGSEA}_{N}$$

Wherein p(CR) is probability of complete response (vs. non-response), and βᵢ are regression coefficients. A genetic algorithm, implemented in R with the glmulti package (1.0.8) was used for feature selection on the 60% training split of the data, using Akaike Information Criterion (AIC) as the objective function. For example, a population size of 100 can be used, with a mutation rate of 0.001, immigration rate of 0.3 and reproduction rate of 0.1.Due to the stochastic nature of genetic algorithms, this was repeated 12,000 times. In other implementations, other population sizes, mutation rates, immigration rates and/or reproduction rates can be used. Moreover, in other implementations, the genetic algorithm can be repeated any number of times. Such processing (e.g., grouping gene signatures) can reduce computing requirements and allow for a more effective selection of a feature vector. In turn, a compute device can select a feature vector(s), train a classifier using the feature vector(s), and/or produce an output using the classifier and/or feature vector(s) much quicker and/or with reduced computing requirements. An example of this kind of workflow is depicted as a workflow (FIGS. 7C and 7D). For randomized-control models (used as comparisons), 2-6 pathways were randomly selected from the pathway compendium as input features (though in other implementations, any other number of pathways can be randomly selected), based on feature frequencies observed in the trained models.

In an alternate embodiment, single sample GSEA scores corresponding to gene signatures that were differentially enriched between CR and NR groups (860, based on an FDR-adjusted p-value of 0.05) can be used to build a logistic regression-based classifier of response status, for example:

$$log ( \frac{p ( C R )}{1 - p ( C R )} ) = {β}_{0} + {β}_{1} \cdot {ssGSEA}_{1} + {β}_{2} \cdot {ssGSEA}_{2} + … + {β}_{N} \cdot {ssGSEA}_{N}$$

Wherein p(CR) is probability of complete response (vs. non-response), and Bi are regression coefficients. To limit the number of input features, Pearson correlation-based hierarchical clustering can be first used to group gene signatures into 25 clusters. This number can be selected to simultaneously maximize and/or improve the inter-cluster correlation and cluster size (FIG. 39). In other implementations, other numbers of clusters can be used. Models can then be randomly seeded with 25 signatures by sampling one per cluster. A genetic algorithm, implemented in R with the glmulti package was then used for feature selection, using Akaike Information Criterion (AIC) with model accuracy as the objective function. Model accuracy can be defined as, for example:

$$Accuracy = ( T P + T N ) / ( T P + T N + F P + F N )$$

Wherein TP, TN, FP, FN refer to true-positive, true-negative, false-positive and false-negatives, respectively. For example, a population size of 100 can be used, with a mutation rate of 0.001, immigration rate of 0.3 and reproduction rate of 0.1. Due to the stochastic nature of genetic algorithms, this was repeated 12,000 times. In other implementations, other population sizes, mutation rates, immigration rates and/or reproduction rates can be used. Moreover, in other implementations, the genetic algorithm can be repeated any number of times. Such processing (e.g., grouping gene signatures) can reduce computing requirements and allow for a more effective selection of a feature vector. In turn, a compute device can select a feature vector(s), train a classifier using the feature vector(s), and/or produce an output using the classifier and/or feature vector(s) much quicker and/or with reduced computing requirements. An example of this kind of workflow is depicted as a workflow (FIG. 7A). FIG. 7A is similar to FIG. 7C. though FIG. 7C includes selecting the top 30 most significant pathways from the extracted significant signatures for seeding a machine learning classifier (rather than calculating per-sample signature enrichment from the extracted significant signatures and clustering signatures into functional modules). FIG. 7B is similar to FIG. 7D. though FIG. 7D includes selecting the top 30 pathways based on significance for input into the genetic algorithm (rather than performing per-sample signature enrichment clustering and selecting clusters to seed a machine learning classifier).

In some implementations. predictive accuracy is assessed using the 40% test split of the data. and model accuracy distributions is compared via Wilcoxon rank-sum tests and visualized as kernel density estimates with manually chosen bandwidths. Immunophenotype classifiers using the same workflow excluding feature selection, with input features being either reported cell frequencies from Fraietta et al., computed cell frequencies from Bai et al. 2022 CITESeq data, or computed cell frequencies from ProjecTILs annotation of Haradhvala et al. data can be developed and/or used.

Binomial tests can be used to assess GSEA overlap in CR vs. NR/PR/RL comparisons between datasets. Starting with the top 28 gene signatures identified as differentially expressed in Fraietta et al. and used to seed the transcriptome classifier, 13/28, 13/28 and 15/28 are significant at a level of p<0.05 in the Bai et al., Haradhvala et al.—Kymriah® and—Yescarta® datasets, respectively. Of the 7548 signatures in the compendium. 1123, 742, and 751 met this level of significance, corresponding to p-values of 6×10⁻⁵, 7×10⁻⁷ and 10⁻⁸.

### scRNAseq (Single Cell RNA Sequencing) Analysis and CITEseq Analysis

Single cell RNA sequencing count data (and associated metadata) was provided by Bai and colleagues (see, e.g., Bai, Z. et al. Single-cell multiomics dissection of basal and antigen-specific activation states of CD19-targeted CAR T cells. J Immunother Cancer 9, e002328 (2021). the contents of which are incorporated by reference herein in its entirety). Single cell RNA sequencing counts and associated metadata for Bai et al 2022 and Haradhvala et al. were retrieved from GEO (GSE197215 and GSE197268, respectively). Gene counts were normalized using Seurat (i.e., software package for quality control, analysis, and exploration of single-cell RNA-seq data: Seurat can enable users to identify and interpret sources of heterogeneity from single-cell transcriptomic measurements, and to integrate diverse types of single-cell data), and cell type labels were subsequently assigned by comparison to flow sorted reference data with SingleR (see, e.g., Aran, D. et al. Reference-based analysis of lung single-cell sequencing reveals a transitional profibrotic macrophage. Nat Immunol 20, 163-172 (2019), the contents of which are incorporated by reference herein in its entirety). While multiple reference datasets were used to confirm the robustness of cell labels, sub-populations were used as defined by Novershtern et al (see, e.g., Novershtern, N. et al. Densely Interconnected Transcriptional Circuits Control Cell States in Human Hematopoiesis. Cell 144, 296-309 (2011), the contents of which are incorporated by reference herein in its entirety) for final analyses. Differential expression analysis was implemented with Seurat using a Wilcoxon rank sum test for each cell type separately, and GSEA performed on the differentially expressed genes using a p-value threshold of 0.05. It is also contemplated that the p-value threshold may be adjusted, and GSEA performed on more or fewer differentially expressed genes (e.g. p-value threshold of 0.1, 0. 01, 0.001, etc.). Single sample GSEA scores were calculated using GSVA (1.40.1) and used without normalization as input features to the classifier.

In an alternate embodiment, the differential expression analysis may employ a median difference test.

For CITEseq-based immunophenotyping, each cell was called as positive/negative based on reference to the associated control antibody tag (Bai et al. 2022).

### Software

Model simulations and analysis were performed using Matlab® R2021a and the SimBiology® toolbox (6.1). Bioinformatics analysis was done on Ubuntu 20.04.3 LTS running R4.1.1 (“Kick Things”). Some packages used in this work involve GSVA (1.40.1) for ssGSEA, fgsea (1.21.2) for GSEA, celldex (1.2.0) for obtaining reference datasets for SingleR (1.6.1) and Seurat (4.1.0), data.table (1.14.2), limma (3.50.3), edgeR (3.34.1), Matrix (1.4.3) and ggplot2 (3.3.6) for data wrangling and visualization.

### EXAMPLE 2

### Model Structure

In this example, T cells (and CAR-T products) to be comprised of 3 functionally distinct cell populations were considered. T memory cells (T_{M}) are capable of long-term self-renewal and immunological memory. T effectors (T_{g}) are responsible for target-mediated cell killing and capable of expansion via undergoing N cell doublings but lack the capacity to self-renew. T effectors can regenerate T memory cells following antigen clearance. After executing N cell divisions, T effectors differentiate to exhausted T cells (Tₓ). lacking both killing potential and proliferative capacity. An antigen sensing switch coordinately regulates the decision of memory cells to self-renew vs. differentiate. the rate of effector proliferation. exhaustion, and the rate of memory cell regeneration from effectors (see Example 1). This represents a biologically sound description of T cell function and regulatory control in response to immunological need. as determined by systemic antigen burden (FIG. 1A).

### Model Parameterization: CLL Patients Treated with Kymriah® and Grouped by Response

Whether the mathematical description of T cell regulatory control could quantitatively capture characteristic CAR-T pharmacokinetic and tumor dynamic profiles. and whether parameter estimates reveal anything about biological underpinnings of clinical variability, was analyzed. Known studies have reported mean PK and tumor dynamic profiles of Chronic Lymphocytic Lymphoma (CLL) patients treated with the Kymriah® (CTL019; a CD19-targeted CAR-T). grouped by Complete Responders (CR). Partial Responders (PR) and Non-Responders (NR). The data (mean±std) was digitized and particle swarm optimization was used to estimate model parameters characterizing the three population archetypes (FIG. 1B). Parameters can be estimated 12 times per patient group. While parameters are non-identifiable (see Tables 25-27), the clinical data was captured with good accuracy (FIG. 9). Accuracy was quantified by computing Pearson correlations of the goodness-of-fits (log-simulations vs. log-data), separated by CAR-T and tumor kinetics for the CR, PR, and NR populations. For the CAR-T pharmacokinetics, median correlation coefficients for the CR, PR, and NR populations are 0.89, 0.88, and 0.76, respectively. The model therefore captures the majority of variance in the PK data for all three groups. For tumor dynamics, the correlations are 0.78. 0.77, and −0.23, respectively. The model accurately captures the CR tumor kinetics. For the PR group, the model describes the dynamics of initial tumor size reduction. For the NR group, the model captured the lack of tumor response. Accuracy was further quantified by comparing the mean squared errors resulting from the set of estimated model parameters to that obtained by random sampling of parameter search space (n=100). P-values for the three groups were all <10-7 (rank-sum test), indicating the improved parameters represent a small segment of parameter space. In some implementations, the tumor growth portion of the model can be minimal (logistic equation) and can capture the overall trends and differences between populations while not capturing aspects of the dynamics. Specifically, in some implementations, both the PR and NR tumor dynamics appear to oscillate up to day -100. then decline.

### Biological Mechanisms Differentiating CR, PR, and NR Populations

To decipher the biological mechanisms underlying the differing patient response profiles. parameter estimates from the three patient populations can be first decomposed into principal components (FIG. 1C). Note the three populations form relatively distinct clusters in parameter space, wherein the X axis depicting PCI (accounting for 35.3% of the variance) separates virtual patients by response, and the Y axis depicting PC2 (accounting for 21.7% of the variance) separates CR and NR groups from PRs. Examining the coefficients of principal component 1 (FIG. 1D). the lowest value (associated with NR) is TK50 (cytotoxic potency of effectors) and the largest positive contributions (associated with CR) is memory and effector cell turnover (proliferation and death rates: μ_{M}, d_{M}, and d_{E2}). That is. in responding patients. CAR-T effectors lyse target tumor cells much more efficiently. and both memory and effector cells cycle at a higher rate. These findings were confirmed by local parameter sensitivity analysis (FIG. 10).

Frequency of memory cells in CAR-T infusion products, as assessed by standard T cell immunophenotyping. can be predictive of clinical response. (see, e.g., (1) Locke et al., (2) Xu et al. Closely related T-memory stem cells correlate with in vivo expansion of CAR.CD19-T cells and are preserved by IL-7 and IL-15. Blood 123, 3750-3759 (2014), the contents of which are incorporated by reference herein in its entirety). This was one conclusion of Fraietta et. al. However, the PCI loadings (FIG. 1D) suggest that cell-intrinsic differences in memory cell function (μ_{M}, d_{M}) rather than frequency (fTm) can sometimes be more important determinants of response. To discern the importance of memory cell frequency vs. function, two experiments were performed. First, the data was fit under the hypothesis that the only difference between CR/PR/NR populations was the composition of the product (frequency of Tm, Te, and Tx cells), while the cell-intrinsic kinetic parameters are conserved (FIG. 42). The model does capture differences in pharmacokinetics and tumor dynamics between the populations, and the inferred CAR-T product composition is consistent with that reported by Fraietta et al. However, the magnitude of differences between the populations cannot be fully explained by Fraietta's hypothesis. That is, CAR-T cell composition as defined by memory and exhausted cell frequencies alone, are sometimes insufficient to explain the variance in clinical activity.

To directly compare the inferred differences in memory cell function between CR/PR/NR groups, dose-ranging study was simulated using purified memory cell populations from CR/PR/NR archetypes (FIG. 43). The CR-memory cells produced robust and dose-dependent CAR-T expansion, persistence, and tumor reduction. while the NR-cells showed very little expansion or anti-tumor activity. and the PR-memory cells display somewhat intermediate function. These results imply that while memory cell frequency in CAR-T infusion products contributes to exposure and response. cell-intrinsic features such as proliferative capacity can sometimes be used to account for the variance clinical outcomes.

### Molecular and Cellular Features Differentiating CR, PR, and NR Populations

To examine the molecular and cellular features underlying these differences in kinetic parameters, RNA sequencing data can be used, wherein pre-infusion CAR-T products can be sequenced and annotated by response category. Differential expression analysis on the CR vs. NR populations revealed biological features (gene signatures) consistent with model predictions (FIG. 11, FIG. 12). Findings from the original report can be confirmed, and additionally find that the CR population is enriched in CD4+ and CD8+ memory cell gene signatures (defined by single-cell sequencing of thymic tissue), and display heightened expression of signatures characterizing T cell proliferation, effector cytokine (interferon) signaling, IL2RB, IL7 and JAK/STAT signaling (as defined by curated pathway databases (see, e.g., (1) BioCarta Pathways. https://maayanlab.cloud/Harmonizome/dataset/Biocarta+Pathways, (2) Gillespie, M. et al. The reactome pathway knowledgebase 2022, Nucleic Acids Res 50. gkab1028-(2021), and (3) Liberzon, A. et al. The Molecular Signatures Database Hallmark Gene Set Collection. Cell Syst 1, 417-425 (2015), the contents of each incorporated by reference herein in its entirety). CAR-T cells from NR patients show heightened p53 and DNA-damage signaling, pathways that may underly the proliferative deficit.

Single-sample Gene Set Enrichment Analysis (ssGSEA) was subsequently used to examine distribution of the pathway and cell signature in individual samples, separated by response. The CR population is significantly enriched in the ‘non-exhausted T cell’ signature (FIG. 2A). This is broadly consistent with model predictions, as exhausted T-cells, by definition, exhibit reduced cytolytic and proliferative capacity. Moreover, the simulated fraction of non-exhausted cells at day-60 (peak of anti-tumor effects) is significantly higher in the CR group (FIG. 2B), while the CAR-Ts from the NR patients rapidly progress to exhaustion (FIG. 32). The simulations also align with other clinical analyses reporting that CAR-T products which fail to expand in vivo show heightened expression of exhaustion markers LAG3 and PD1.

CRs are differentially enriched in both CD8+ and CD4+ memory T cell signatures (FIG. 2C, D). The CR population also shows heightened IL2RB and IL7R signaling (FIG. 2E, F). These pathways converge on canonical JAK/STAT cascade, indicating the CR cell products may show heightened sensitivity to these critical cytokines. IL2 and IL7 are known components of CAR-T expansion media, and peak serum IL-7 concentration may be predictive of CD19 CAR-T exposure and progression-free survival (PFS). While the results shown in FIG. 2 are statistically significant. the ssGSEA distributions overlap between response categories. Thus, in some implementations, none of the gene signatures assessed are used as univariate classifiers of patient response. Also note that gene set enrichment on bulk-sequencing data may not always resolve cell population frequencies or discern between transcriptionally similar vs. covarying cell types (FIG. 33). For example, CR products may have higher frequencies of CD4+ and CD8+ memory cells. or may contain equivalent cell frequencies, but with more memory-like' transcriptomes.

To assess model predictions at deeper resolution, a known single cell RNA sequencing study of pre-infusion autologous CD19 CAR-T products (bearing the 4-1BB costimulatory domain, analogous to Kymriah®) from 12 patients with acute lymphoblastic leukemia, five complete responders (CR), two non-responders (NR) and five patients who relapsed (RL), can be used. Cell type labels can be assigned by mapping expression profiles of the 31,105 individual cells to T-cell sub-populations from tumor-infiltrating lymphocytes and from healthy donors using two complementary methods (FIG. 3A and FIG. 34A,B). Cells from the two products form clearly distinct clusters in UMAP (Uniform Manifold Approximation and Projection) space. UMAP is a dimensionality reduction algorithm based on manifold learning techniques and topological properties of data that can be used in some implementations to construct a high-dimensional graph representation of data, then optimize a low-dimensional graph to be as structurally similar as possible and create the UMAP space (the two-dimensional space between UMAP_1 (x-axis) and UMAP_2 (y-axis)). Consistent with the bulk sequencing data. frequencies of both CD8+ and CD4+ central memory cells (the most primitive annotated cell type) can be enriched in the CR compared to the NR patient (FIG. 3B). While the majority of cells are annotated as T_{EM} (effector-memory), the NR and RL patients showed a significant enrichment (p=0.04,one-sided t-test) for exhausted T cells (FIG. 3D), as well as CD8+ EMRA cells (effector memory cells which re-express CD45RA: FIG. 34C). Thus, regardless of cell annotation method, CAR-T products leading to non-or non-durable-responses show higher frequencies of terminally differentiated CD8+ effector sub-populations. Cells using a CART dysfunction' signature, characteristic of functionally exhausted CAR-T cells were annotated with reduced proliferative and cytotoxic capacity. Visually, the dysfunction signature segregates into the NR/RL categories, consistent with model-predicted functional differences (FIG. 3C).

To interrogate cell-intrinsic differences underlying response, differential gene expression analysis on T sub-cell populations was performed, followed by pathway enrichment for select functional gene signatures (FIG. 3E). As a control, differences between cells annotated as exhausted vs. non-exhausted were first assessed. Exhausted cells are enriched in ‘exhausted T cell’ and ‘CART dysfunction’ signatures, as well as ‘P53 signaling’ and ‘negative regulation of T cell proliferation’. Conversely, non-exhausted cells are enriched for ‘T cell proliferation’ and ‘early memory’ signatures. The CR vs. NR/RL population analyses reveal a similar, consistent pattern. Focusing either on effector-memory or non-specific CD8+ T cells. the NR/RL populations display characteristic features of exhaustion; heightened CART dysfunction and P53 signaling, and reduced T proliferation and early memory signatures. Similar results were produced from the equivalent analysis performed using cell annotations derived from healthy donors (FIG. 34D).

In sum, the single cell data confirms predictions from the model in a different indication (ALL): infusion products associated with non-durable response show heightened frequency of exhausted cells, and the effector-memory populations display intrinsic deficits in proliferative and functional capacity.

In an additional or alternate embodiment, a known single cell RNA sequencing study of pre-infusion autologous CD19 CAR-T products (bearing the 4-1BB costimulatory domain, analogous to Kymriah®) from two patients with acute lymphoblastic leukemia, one responder and one non-responder, can be used. Cell type labels can be assigned by mapping expression profiles of the 4879 individual cells to T-cell sub-populations sorted from healthy donors (FIG. 37A). Cells from the two products form clearly distinct clusters in UMAP space. Consistent with the bulk sequencing data, frequencies of both CD8+ and CD4+ central memory cells (the most primitive annotated cell type) can be enriched in the CR compared to the NR patient (FIG. 37B). The NR patient showed a significant enrichment for CD8+ EMRA cells (effector memory cells which re-express CD45RA). This is a differentiated sub-population with markedly reduced proliferative capacity, consistent with model-predicted reduced turnover rate of both memory and effector cells in the NR class.

Continuing with an alternate embodiment, to interrogate cell-intrinsic differences underlying response, differential gene expression analysis was performed on matched T cell populations between the CR vs NR patient, followed by pathway enrichment for select exhaustion, proliferation, and inflammatory pathway signatures (FIG. 37C). Consistent with bulk sequencing data and model predictions, cell populations from the CR patient appeared significantly more non-exhausted, and conversely cells from the NR patient exhausted. Cell populations from the CR patient were also consistently enriched for a T cell proliferation signature. While differences in interferon signaling were not significant, the inflammatory signaling pathways TNFa and NFκB were heightened in the CR cells. Differences between the CD8+ EM vs. EMRA sub-populations can also be compared. The CD45RA+ cells showed increased exhaustion, reduced proliferation, and reduced inflammatory signaling (i.e., cytotoxic potency).

In some implementations, to deconvolute the role of cell frequency vs. function in mediating response, two recently published clinical studies were leveraged containing single cell RNA sequencing data of pre-infusion, autologous CD19 CAR-T products matched with clinical outcomes. Bai et al. 2021 report data for 12 patients with acute lymphoblastic leukemia (ALL) treated with a CD19 CAR-T product analogous to Kymriah®; five complete responders (CR), two non-responders (NR) and five patients who relapsed (RL). Haradhvala et al. report data for 32 patients with large B-cell lymphoma (LBCL) treated with either Kymriah® (n=13) or Yescarta R (n=19). Of the Kymriah®-treated group, there were six CR and seven NR, and for the Yescarta R-treated group, eleven CR, one PR, and seven NR.

Examination of UMAP projections of the three datasets (Kymriah® in ALL. Kymriah® in LBCL and Yescarta® in LBCL) reveal some separation of response categories in transcriptome space. particularly in ALL (FIG. 40A,D,G). To assess whether response separation is attributable to differences in T cell composition. cell type labels were assigned by mapping expression profiles of the individual cells to annotated tumor-infiltrating lymphocyte populations via ProjecTILs. The majority of CD8+ cells in all three datasets are classified as T effector-memory (Tem) or T exhausted (Tex), but there are little to no consistent differences in composition by response category (FIG. 44A-C). For example, the frequency of cells annotated as exhausted is higher in the NR/RL categories as compared to CR in the ALL data (p<0.05, mean 4.4% vs. 8.7% respectively; FIG. 40B, E, H). However. this pattern does not always hold for the LBCL data, and the modest effect size can be insufficient to account for the disparity in clinical outcomes. The CITESeq antibody tag data provided by Bai et al. 2022 was used to assign early memory (Tmem: CD8+CD45RO-CD27+) and exhausted (CD8+PD1+) cell annotations by immunophenotype, reported to be predictive of response in CLL (Fraietta et al.) While exhausted cell annotations by ProjecTILs and immunophenotype were concordant (6.7% vs. 5.9% of total cells), cell frequencies did not differ substantially by response category in ALL (FIG. 44D,E).

To probe cell-intrinsic function. cells were annotated using a CAR-T dysfunction™ signature. characteristic of functionally exhausted CAR-T cells with reduced proliferative and cytotoxic capacity (Good et al.) Visually, the dysfunction signature is dispersed throughout response categories and not restricted to exhausted regions (FIG. 40G, H I). Interrogating cell-intrinsic functional differences at a deeper resolution. differential gene expression analysis was performed on T sub-cell populations (annotated both by transcriptome and immunophenotype), followed by pathway enrichment for select gene signatures (FIG. 40J). As a control, differences were first assessed between cells annotated as exhausted vs. non-exhausted. Exhausted cells are consistently enriched in the CAR-T Dysfunction signature across datasets. while the ‘exhausted T cell’ and ‘P53 signaling’ signatures appear specific to the ALL-exhausted cells. Conversely, non-exhausted cells show disparate enrichment for the ‘early memory T cell’ signature, as well as cytokine production and inflammatory response signatures, hallmarks of T cell functional potency.

Comparing cell populations from the CR vs. NR/PR/RL categories reveals a consistent pattern across datasets. Focusing either on effector-memory or early memory (CD8+CD45RA-CD27+) subsets, the NR/PR/RL groups display characteristic features of exhaustion. In particular, the CAR-T Dysfunction signature is consistently heightened. The CR cell populations conversely show increased expression of early memory and/or T cell functional signatures (cytokine production and inflammatory response). That is, memory and effector cell populations from CAR-T products resulting in CR appear more functional or ‘memory-like’, while the same cell populations from NR/PR/RL categories appear more exhausted. The single cell data thus confirms inferences from the model in separate indications (ALL and LBCL): CAR-T infusion products associated with non-durable response display deficits in proliferative and functional capacity intrinsic to memory and effector cell populations.

### Cell-Intrinsic Attributes Predictive of CAR-T Response can be Inferred from Pre-Infusion Product Transcriptomes

If CAR-T response is product-rather than host-intrinsic, it can be reasoned that the differences in pre-infusion product transcriptomes may be predictive of response. Moreover, comparing response classifiers based on cell-intrinsic function (transcriptome) vs. cell composition (T cell phenotype) could help elucidate which product-intrinsic feature is more clinically relevant. While it may sometimes be the case that none of the individual gene signatures assessed would accurately classify response, bulk RNAseq data can be used to develop a multivariate transcriptome classifier. For example, in an implementation. starting with the 30 pathways that were differentially expressed between the CR vs. NR groups (FDR-adjusted p-value <0.05), a logistic regression-based classifier using a genetic algorithm for feature selection can be used (see, e.g., FIG. 13).

The resultant model was able to predictively distinguish CAR-T products from CR vs. NR patients, with an average accuracy of 83% based on a train/test split of 60:40(FIG. 4A, FIG. 41A). This result was compared to two control scenarios: a naïve classifier (which randomly calls R/NR based on sample frequencies in the dataset), and one where input features are selected at random from the remnant compendium of pathways (FDR-adjusted p-value >0.05). The trained model performed significantly better than each of these control cases (p<10⁻¹⁵ for each). The PR samples (not used in training) were called as mid-way between CR and NR, which could be real biology, or simply failure of the classifier on these distinct samples (FIG. 13).

To confirm that these findings were replicable, the discussed framework was applied to the single cell data from Bai. Of the 30 pathways identified as significantly different in CART product transcriptomes between CR vs. NR in CLL, 6 are coordinately differentially enriched between CR vs. NR/RL patients in ALL: E2F Targets (Hallmark). Estrogen Response Late (Hallmark), Myc Targets VI (Hallmark), Cell Cycle (Reactome). Cell Cycle Mitotic (Reactome), and DNA Replication (Reactome) (See Supplemental Dataset A). This degree of overlap is highly significant (p=3×10⁻⁸, binomial test), indicating shared biological mechanisms associated with robust efficacy between these diseases. The same 30 pathways can thus be used as input features to the logistic regression classifier (see, e.g., FIGS. 7C and 7D), using pseudo-bulked ssGSEA scores. Again, the resulting accuracy was compared to a random pathway control case and a naïve classifier. The trained classifier significantly out-performs these cases (p<10⁻⁸ in both) with an average accuracy of 75%, demonstrating cross-dataset predictive accuracy. Note, however, that more or less than 30 pathways can be used as input features in other implementations.

In summary, this shows that response is largely predetermined by the cellular composition of the CAR-T product, and response can be accurately predicted from pre-infusion transcriptomes. For example, in an embodiment, response to Kymriah® in two different indications (CLL and ALL) is at least partially predetermined by the cellular composition of the CAR-T product, as response can be accurately predicted from pre-infusion transcriptomes, and the transcriptional features predictive of response are shared across the two disease indications.

In an additional or alternate implementation, starting with the 857 pathways that were differentially expressed between the CR vs. NR groups, an alternate logistic regression-based classifier using a genetic algorithm for feature selection can be used (see e.g., FIG. 7A, FIG. 7B, FIG. 39).

Continuing with the additional or alternate implementation, the resultant model was able to predictively distinguish CAR-T products from CR vs. NR patients, with an average cross-validated accuracy of 94% (FIG. 38A). As the classifier can be trained on CR vs. NR groups, how the model would classify PR and PRID samples was queried (FIG. 38B). These were inconsistently annotated as essentially mid-way between R vs. NR classes, recapitulating the clinical annotation.

In some implementations, classifiers were trained and assessed using the early memory (CD8+CD45RO-CD27+) and exhausted (CD8+PD1+LAG3+) cell frequencies as reported in Fraietta et al. (FIG. 44D). The resulting accuracies (80% and 83%, respectively) are significantly better than chance, but less so than that achieved using functional transcriptomes (p<¹⁰⁻¹⁵ and p=6×10⁻¹¹, respectively). The gene signature panel thus reveals clinical functionality to an extent not apparent from immunotyping, implying that transcriptomes yield more value as CAR-T product characterization assays than current best practice flow cytometry panels.

To assess whether these findings translated across datasets and indications, the same workflow was applied to pseudo-bulked single cell data from Bai et al. 2022 (Kymriah® in ALL) and Haradhvala et al. (Kymriah® and Yescarta® in LBCL) For the Bai et al. 2022data (Kymriah® in ALL) the accuracy of classifying CR vs. NR/RL groups using the 28 gene signature panel was compared to a bivariate classifier trained using the early memory (CD8+CD45RO-CD27+) and exhausted (CD8+PD1+) immunophenotype frequencies calculated from CITEseq antibody tags (FIG. 44D). Median accuracy of the transcriptome classifier was 80%, less (as expected) than before, but better than that achieved by T cell immunophenotyping (47%, p<10⁻¹⁵; FIG. 41B). Similarly. predictive accuracy was assessed using the LBCL data from Haradhvala et al. separately for Kymriah® and Yescarta®. As no immunophenotype data was provided, the transcriptome classifier was compared to bivariate classifiers based on estimated T effector-memory (Tem) and exhausted cell (Tex) frequencies from ProjecTILs (Andreatta et al.) annotations (FIG. 44B, C). Median predictive accuracy of the transcriptome classifier was 80% and 71% for Kymriah® and Yescarta® respectively, outperforming T cell phenotype-based classification in both cases (60% and 67%, p<10⁻¹⁵, FIGS. 41C and D). As an additional control. the classifier was seeded with ‘random’ pathways by sampling from the compendium of gene signatures which were not differentially expressed between CR vs. NR groups in the CLL data (FDR-adjusted p-value >0.05, see Example 1 and FIG. 45). The resulting accuracies were either slightly better or indistinguishable from chance (the ‘null’ model), and all significantly less accurate than predictions arising from the 28 gene signature panel.

To condense the inner workings of the transcriptome-classifier into interpretable patterns, a CAR-T response scorecard was created (FIG. 41E). This summarizes GSEA on the 28 select pathways and frequency of inclusion in the 2.500 trained models across each of the four datasets. There is variance in the directionality and statistical significance of the signatures between datasets, as would be expected. These represent different diseases. CAR-T products and platforms, and that the data was generated by independent groups. However, the overlap is greater than would be expected by chance (p<10⁻⁵ for all, see Example 1). For example, the Yescarta R-LBCL scorecard is visually distinct from the three Kymriah®-scorecards and the resulting model predictions are corresponding less accurate. This suggests distinct vet overlapping biology underlying response between the two products.

In summary, response to two separate CD19 CAR-T therapy products (Kymriah® and Yescarta®) in three indications (CLL, ALL and LBCL) is at least partially predetermined by functional attributes of the CAR-T infusion product. These functional attributes are shared across the four datasets to varying extents, revealed through gene signatures, and not fully apparent from T cell immunophenotyping.

### EXAMPLE 3

### Explaining Inter-Patient Variability in Kymriah® Pharmacokinetics

The pharmacplometocs pf Kymriah® and other CAR-T products tested in clinical trials show high inter-patient variability (e.g., with AUCs spanning three orders of magnitude) Whether the mechanism-based model described herein is explanatory of this variability was tested. Specifically, whether a mixture of the three patient archetypes (CR/PR/NR), combined with reported variation in administered dose and initial tumor burden are sufficient to quantitatively account for the observed variance in exposure.

Simulations of the CR/PR/NR pharmacokinetic profiles were overlaid with that of Kymriah®. While these were different patient populations (CLL vs. B-ALL). the pharmacokinetics are conserved between these two indications. Visually, the CR/PR/NR profiles correspond roughly to the top-quartile, median, and bottom 5% of exposure (FIG. 5A). Thus, the CR/PR/NR population archetypes cover much of the pharmacokinetic variation, but do not fully account for individual patient variability as they were fit to population means.

The effect of variability in dose and tumor burden was assessed using a virtual population approach. Virtual populations (n=1000) were defined by Monte Carlo sampling across the parameter sets, while randomizing dose and tumor burden within reported ranges, either alone or in combination by log-uniform sampling.

The simulated exposures (AUC) for these virtual populations span the inter-individual variability of Kymriah® (10¹10⁴ cells·day/μL; FIG. 5B). Variance in either dose or tumor burden is sufficient to cover, and roughly match the reported variance of exposure within the CR/PR/NR populations. That is, while the model was fit to population mean data assuming fixed tumor burden and dose, relaxing either of these inputs is sufficient to account for reported variance. Similar results are produced by examining the Cmax (FIG. 5C). Grid simulations were used to assess how tumor burden and dose drive exposure and tumor response (FIG. 14), revealing a non-linear relationship that may contribute to the clinical variance. Tumor AUC was found to increase with initial tumor burden and decreases with initial CAR-T dose (FIG. 14). Cmax exhibits a more complex relationship, increasing with initial CAR-T dose but peaking for intermediate tumor burdens. This non-linear interaction between tumor burden and CAR-T dose may contribute to the clinically observed variability.

### Predicted Covariates of Response: Cmax and Tumor Burden

Whether the virtual populations could predict a priori the reported statistical relationships between cell expansion and tumor burden to clinical response was examined. Analysis of response covariates to YescartaR in large cell B-cell lymphoma (LCBCL) identified the ratio of CAR-T expansion to initial tumor burden (i.e., Cmax/B0) as the strongest correlate of durable response. The same result was reported for overall survival in B-ALL. indicating this is a conserved feature across indications. The median pharmacokinetics and population variance of Yescarta® are similar to Kymriah® (FIG. 35).

Focusing on the virtual CR population, a response was defined by the B-cell AUC, set to 10⁴ cells*day/μL (the minimum observed for the virtual PR population). A logistic regression model linking response to initial tumor burden (B0), Cmax. or the ratio as predictors (FIG. 5D-F) was used. The equivalent logistic curves from Yescarta® were digitized and overlaid by normalizing the x-axes. The results are qualitatively consistent with the clinical data. in that these covariates are predictive of response.

To assess whether these predictions emanate directly from the model structure or necessitate model training, a ‘control’ virtual population can be defined by random sampling of parameter space (N=1000). This control population did not reproduce the same findings. emphasizing the need for appropriate training data to make accurate predictions.

### Dose-Response Implications: Multiple Myeloma Patients Treated with Abecma® (BCMA-CART)

To better understand the relationship between dose, Cmax and tumor response, the modelling framework can be applied to a phase I/II dose escalation study of Abecma R (BB2121. Idecabtagene Vicleucel), a BCMA-targeted CAR-T approved for the treatment of multiple myeloma. Particle swarm optimization can be used to estimate model parameters characterizing the pharmacokinetic and tumor dynamics (FIG. 6A, B). While parameters are non-identifiable (see Table 3 for parameters), both were captured with good accuracy (FIG. 15), and simulations recapitulate the relationship between Cmax/B0 and tumor response identified in FIG. 5F for Kymriah® and Yescarta® (FIG. 36).

TABLE 3
Five parameter sets estimated for Abecma ®.

| Parameter | Set 1 | Set 2 | Set 3 | Set 4 | Set 5 |
|---|---|---|---|---|---|
| B50 | 9.99E+09 | 2.22E+09 | 9.99E+09 | 7.39E+08 | 3.76E+09 |
| μ_{B} | 0.009589 | 0.005016 | 0.005002 | 0.005001 | 0.02384 |
| kₖᵢₗₗ | 0.443294 | 0.292815 | 0.528402 | 0.251047 | 0.26201 |
| fₗₒₛₛ | 1 | 1.000027 | 1 | 1.004481 | 1.002751 |
| TK50 | 5.06E+08 | 9.32E+08 | 9.6E+08 | 2.65E+08 | 3.14E+08 |
| kt | 0.484273 | 0.74767 | 0.468533 | 0.63864 | 0.571639 |
| k_{B1} | 0.336043 | 0.001948 | 0.364665 | 0.981316 | 0.568101 |
| k_{B2} | 1 | 0.365705 | 0.999394 | 0.992798 | 0.999835 |
| μ_{M} | 0.001 | 0.115701 | 0.001011 | 0.001028 | 0.001067 |
| km | 2.994876 | 1.791614 | 3 | 0.718047 | 1.895636 |
| fₘₐₓ | 0.630801 | 0.98522 | 0.696398 | 0.54641 | 0.938307 |
| ke | 0.370319 | 0.318038 | 0.35794 | 0.209473 | 0.434523 |
| μ_{Ε} | 0.728744 | 0.79872 | 0.628718 | 0.400997 | 0.583925 |
| N | 5.999439 | 5.999828 | 5.999855 | 5.998935 | 5.996735 |
| kₑₓ | 0.002029 | 0.001 | 0.003012 | 0.309727 | 0.514441 |
| d_{M} | 0.163137 | 0.89411 | 0.159431 | 0.057425 | 0.025341 |
| d_{E1} | 0.001032 | 0.001027 | 0.001053 | 0.003952 | 0.001 |
| d_{E2} | 0.001441 | 0.085212 | 0.00121 | 0.001 | 0.05111 |
| d_{X} | 0.02039 | 0.019565 | 0.020464 | 0.200266 | 0.162584 |
| Bₘₐₓ | 9.99E+10 | 9.97E+10 | 4.96E+10 | 7.14E+10 | 9.76E+10 |
| fraction_{TM} | 2.929435 | 2.99011 | 1.920162 | 0.2 | 2.99762 |
| fraction_{TE1} | 1 | 0.101232 | 0.972766 | 0.038335 | 0.001001 |
| fraction_{TE2} | 2.969084 | 1.968508 | 2.955973 | 2.915625 | 2.093411 |
| fraction_{TX} | 1.025226 | 1 | 1.044959 | 1.000313 | 1.002828 |
| kx | 9.998224 | 9.96492 | 9.984916 | 9.899459 | 9.985704 |
| r_{M} | 30.03598 | 30 | 30.05013 | 30.15663 | 30.06973 |
| kr | 5.000042 | 5 | 5.010552 | 5 | 5.000211 |

The simulations yield insight into the effects of CAR-T dose on T cell population dynamics (FIG. 6C-E). The lowest dose (50 million cells) was incapable of tumor reduction and resulted in a predominance of exhausted T cells and gradual loss of memory cells. The highest dose, for which the greatest degree of tumor reduction was observed, produced the opposite response, with minimal exhaustion and a high fraction of memory cells. This is analogous to changes in T cell composition following acute vs. chronic infection and provides mechanistic underpinning to the covariates identified above. That is, at an insufficient the Cmax: tumor burden ratio, either due to low dose or expansion capacity. the infused CAR-T population will exhaust before clearing tumor.

To assess the predictivity of the model, simulations were compared against data from the phase 2 study, wherein patients were treated at doses of 150, 300 and 450 million cells, and tumor dynamics (BCMA levels) were monitored out to a year (FIG. 6F, G). While both the PK and tumor dynamics are moderately under-predicted, the profiles are captured with reasonable accuracy. That is, the phase 2 data (150-450 million cell doses) fall between the simulated 150 and 450 million cell doses with similar dynamics. This is notable for the tumor dynamics, given that the model was trained on data going out to 2 months, while predictions are extrapolated out to a year.

Some known clinical studies have noted that robust cell expansion following CAR-T infusion is a prerequisite for clinical efficacy. An inability to predictively control the pharmacology of CAR-Ts thus may limit their clinical utility. Mechanism-based mathematical models present a path forward. When trained using appropriate datasets, such models enable both the inference of underlying biological principles governing response, the ability to generate quantitative predictions, and ultimately guide therapeutic design. It was hypothesized that the principles governing T cell dynamics during infection also govern the pharmacology of CAR-Ts and tested this using a conceptually simple mathematical model of T cell regulatory control. based on an analogy to a toggle switch. The model was trained using available clinical pharmacokinetic and tumor dynamic data, yielding biological insights and clinical predictions, some of which have been confirmed.

First, CAR-T expansion. persistence and anti-tumor response can be driven by cell-intrinsic rates of turnover of memory T cell populations and cytotoxic potency of effectors. Using bulk gene expression data, it was found that enrichment of memory cell signatures, heightened proliferative and inflammatory signaling and lack of exhaustion markers in pre-infusion CAR-T products correlates with response. Single cell sequencing data from two additional disease indications and an additional CD19 CART product confirmed these differences between CR and NR archetypes are intrinsic to memory cell function rather than frequency in the infusion products. CAR-T products resulting in non-durable response show deficits in proliferative and functional capacity characteristic of T cell exhaustion and terminal differentiation, even within immunophenotypically indistinguishable memory and effector cell populations. These functional differences were inferred from models and/or methods described herein and confirmed via expression of a ‘CAR-T dysfunction’ gene signature. CAR-T expansion following infusion (e.g., Cmax) may represent an in vivo readout of memory T cell proliferative capacity.

Response categories may be accurately predicted using pre-infusion product transcriptomes in three indications (CLL, ALL and LBCL) and two CD19-targeted products (Kymriah® and Yescarta®). Moreover, transcriptome profiles reveal functional attributes not apparent from standard immunophenotyping. and these attributes are shared to varying extents among the datasets examined. In some implementations, the memory/exhaustion phenotypes identified as predictive of response in CLL did not translate to ALL, while the gene signature panel did. Moreover, if pre-infusion product transcriptomes are predictive of response, this could imply that these pharmacological archetypes are intrinsic to the infusion product, and thus CAR-T efficacy could be improved through product design.

A simple, easily implemented molecular signature for efficacious (CR-like) CAR-T products could be desirable for guiding optimization studies. The CAR-T response scorecard (FIG. 41E) reveals transcriptional features which are shared to varying extents between the four datasets. While there are statistically significant similarities, disparate molecular mechanisms appear to coordinately mediate clinical outcomes between the three datasets, and particularly between the two products (Yescarta® vs. Kymriah®). In some implementations, this scorecard can serve as a useful tool for CAR-T product optimization. The pathways selected, however, are derived from the first dataset examined (Kymriah®) in CLL). It is thus a visual representation of the workflow, rather than a comprehensive map of features shared consistently across datasets. Additionally, the different shades represent group-level differential pathway enrichment, while the classifiers were trained on single-sample GSEA scores. This compression loses information about the variance within sample groups. which may be important for multivariate classification. The algorithms and methods may thus select signatures which do not vary significantly at the group-level (i.e., intermediate score (e.g., between range 4502 and 4504 at FIG. 41E), high frequency (e.g., at least 70 at FIG. 41E)) but nonetheless contain information. Moreover, many of the signatures make sense biologically (e.g., JAK/STAT signaling, exhausted T cell), while others less so (e.g., EMT, xenobioti—many of the signatures are manually curated with inconsistent degrees of validation, and gene lists will overlap between biological processes.

Pharmacologic archetype. combined with variability in CAR-T cell dose and initial tumor burden accounts for the inter-patient variability in exposure observed in clinical trials of Kymriah®. The ratio of CAR-T expansion (Cmax) to initial tumor burden (B0) quantifies whether the cell product infused is capable of clearing tumor, a de novo prediction from the model observed in known studies of Yescarta®. Mechanistically. cell doses insufficient to clear tumor result in exhaustion of the CAR-Ts, while sufficient doses lead to regeneration of memory populations.

Controlling the clinical variability in cell dose and initial tumor burden may be desirable. Cell dose has been previously defined by whatever comes out of the manufacturing process, and initial tumor burden the remanent cancer cells following lymphodepleting chemotherapy, both of which are highly variable between patients. Given consistent quality CAR-T products (e.g., those displaying a CR-class transcriptional signature), model simulations can be used to define patient-specific doses based on tumor burden (B-cell counts) to achieve an optimal balance between maximizing tumor reduction and minimizing Cmax-associated toxicity (FIG. 14), in some implementations. FIG. 8 provides an overview of an example workflow using model simulations to optimize treatment.

Note that. while variability in CAR-T dose and tumor burden may be sufficient to explain the observed variance in exposure, additional host-intrinsic factors may be involved (e.g., response to lymphodepletion).

The CR vs. NR archetype may be a product-intrinsic property. In some instances, product-intrinsic may mean that clinical response is sufficiently predictable by properties of the infusion product. These properties (e.g., memory vs. exhaustion phenotype) may in turn be pre-determined by the patient's immunological state—a host-intrinsic property. Note that some of the model parameters may integrate some aspects of more than one property. Cytotoxic potency (TK50), for example, appears to be a cell intrinsic parameter. However, this lumps together multiple cellular processes: CAR and antigen expression, CAR-antigen binding kinetics, intracellular signal transduction, and engagement of cytotoxic machinery. These processes are in turn regulated by systemic cytokines and cell-cell interactions. A similar case could be made for other model parameters. Thus, while variability in CAR-T dose and tumor burden are sufficient to explain the observed variance in exposure, the inclusion of additional host-intrinsic factors may extend the model's utility. Tumor-intrinsic signaling and response to lymphodepletion are two examples. Both have been shown to mediate CART expansion and tumor response, as cytokine-mediated interactions between CAR-Ts, host T cells and tumors likely mediate cell-intrinsic differences.

The described model formulation has at least a few differences compared to known predator-prey structure to address the four requisite properties and incorporate fundamental T cell biology. Borrowing from the stem cell field, each T memory (T_{M}) cell division was encoded as a fate choice between self-renewal and differentiation, driven by tumor antigen (B_{A}). CAR-T differentiation and expansion thus occur at the expanse of depleting the pool of memory cells. In some implementations, effector cells (T_{g}) do not self-renew; but rather undergo a fixed number (N) of divisions. This can address the unlimited CAR-T expansion capacity found in some known predator-prey models. Accounting of memory cell self-renewal vs. differentiation also provides a mechanism by which chronic antigen stimulation (or alternatively, insufficient CAR-T dose relative to tumor size) drives exhaustion. If tumor cells cannot be cleared sufficiently to reduce systemic antigen burden below a defined threshold (B50), Tm cells will continually differentiate until the pool of long-term memory cells is depleted.

An exhausted T cell state is also included. An exhausted T call state can, in some implementations, capture the divergence between CAR-T pharmacokinetics and cytotoxic function, particularly in partial and non-responding patients (explored in detail below).

### EXAMPLE 5

### Model Structural Assessment

To systematically assess the model topology, a series of variants with alternate T cell population structures were defined, for example:
1. T_{E} population only (effector state)
2. T_{M} and T_{E} (memory and effector states), no Tₓ
3. T_{E} and Tx (effector and exhausted states)
4. T_{M}, T_{g}, and Tₓ states, but without effector to memory differentiation (r_{M}=0)
5. Inclusion of additional naïve (T_{N}) state to the original model.

FIG. 28 illustrates these model structures.

Note the original (complete) describes 4 sub-populations regulated by antigen exposure via the ODEs, for example:

$$\frac{d {T}_{M}}{d t} = 2 \cdot {μ}_{M} \cdot {f}_{max} \cdot ( 1 - \frac{{B}_{A}^{k m}}{B 5 {0}^{k m} + {B}_{A}^{k m}} ) \cdot {T}_{M} + {r}_{M} \cdot ( 1 - \frac{{B}_{A}^{k r}}{B 5 {0}^{k r} + {B}_{A}^{k r}} ) \cdot {T}_{E 2} - {d}_{M} \cdot {T}_{M} ,$$

$$\frac{d {T}_{E 1}}{d t} = 2 \cdot {μ}_{M} \cdot ( 1 - {f}_{max} \cdot ( 1 - \frac{{B}_{A}^{k m}}{B 5 {0}^{k m} + {B}_{A}^{k m}} ) ) \cdot {T}_{M} - {μ}_{E} \cdot ( \frac{{B}_{A}^{k e}}{B 5 {0}^{k e} + {B}_{A}^{k e}} ) \cdot {T}_{E 1} - {d}_{E 1} \cdot {T}_{E 1}$$

$$\frac{d {T}_{E 2}}{d t} = {μ}_{E} \cdot {2}^{N} ( \frac{{B}_{A}^{k m}}{B 5 {0}^{k m} + {B}_{A}^{k m}} ) \cdot {T}_{E 1} - {k}_{e x} ( \frac{{B}_{A}^{k x}}{B 5 {0}^{k x} + {B}_{A}^{k x}} ) \cdot {T}_{E 2} - {r}_{M} \cdot ( 1 - \frac{{B}_{A}^{k r}}{B 5 {0}^{k r} + {B}_{A}^{k r}} ) \cdot {T}_{E 2} - {d}_{E 2} \cdot {T}_{E 2}$$

$$\frac{d {T}_{X}}{d t} = {k}_{e x} ( \frac{{B}_{A}^{k x}}{B 5 {0}^{k x} + {B}_{A}^{k x}} ) \cdot {T}_{E 2} - {d}_{X} \cdot {T}_{X} .$$

For model variant 1, the single effector compartment is described, wherein proliferation/self-renewal is driven by antigen, for example:

$$\frac{d {T}_{E 2}}{d t} = {μ}_{E} ( \frac{{B}_{A}^{k m}}{B 5 {0}^{k m} + {B}_{A}^{k m}} ) \cdot {T}_{E 2} - {d}_{E 2} \cdot {T}_{E 2} ,$$

For model variant 2, the full model is employed, but sets kₑₓ=0 such that no exhausted T cells are generated.

For model variant 3, a version of variant 1 is employed, wherein effectors both proliferate/self-renew and transit to exhausted cells in an antigen-dependent manner.

$$\frac{d {T}_{E 2}}{d t} = {μ}_{E} ( \frac{{B}_{A}^{k m}}{B 5 {0}^{k m} + {B}_{A}^{k m}} ) \cdot {T}_{E 2} - {k}_{e x} ( \frac{{B}_{A}^{k x}}{B 5 {0}^{k x} + {B}_{A}^{k x}} ) \cdot {T}_{E 2} - {d}_{E 2} \cdot {T}_{E 2}$$

$$\frac{d {T}_{X}}{d t} = {k}_{e x} ( \frac{{B}_{A}^{k x}}{B 5 {0}^{k x} + {B}_{A}^{k x}} ) \cdot {T}_{E 2} - {d}_{X} \cdot {T}_{X} .$$

For model variant 4, the oroiginal set of model equations is emplohed, but sets T_{M}=0 such that memory cells cannot arise from effectors. This variant thus assesses, in some implementations, whether a teleological de-differentiation reaction is necessary, given the limited number of T cell states considered for parsimony.

For model variant 5, a naïve T cell compartment (T_{N}) preceding the memory compartment is included, as per canonical T cell differentiation hierarchy. These cells proliferate and differentiate to memory T_{M} cells in an antigen-dependent manner, via the equation, for example:

$$\frac{d {T}_{N}}{d t} = 2 \cdot {μ}_{N} \cdot {f}_{N} \cdot ( 1 - \frac{{B}_{A}^{k n}}{B 5 {0}^{k n} + {B}_{A}^{k n}} ) \cdot {T}_{N} - {d}_{N} \cdot {T}_{N}$$

TN cells differentiate into the memory cell compartment, such that the TM balance equation is now, for example:

$$\frac{d {T}_{M}}{d t} = 2 \cdot {μ}_{N} ( 1 - {f}_{N} \cdot ( \frac{{B}_{A}^{k n}}{B 5 {0}^{k n} + {B}_{A}^{k n}} ) ) \cdot {T}_{N} + 2 \cdot {μ}_{M} \cdot {f}_{max} \cdot ( 1 - \frac{{B}_{A}^{k m}}{B 5 {0}^{k m} + {B}_{A}^{k m}} ) \cdot {T}_{M} + {r}_{M} \cdot ( 1 - \frac{{B}_{A}^{k r}}{B 5 {0}^{k r} + {B}_{A}^{k r}} ) \cdot {T}_{E 2} - {d}_{M} \cdot {T}_{M}$$

This introduces five additional free parameters into the model:
μ_{N}: the naïve T cell proliferation rate
f_{N}: the naïve T cell probability of self-renewal
k_{N}: the Hill exponent linking antigen exposure to naïve T cell proliferation
d_{N}: the naïve T cell death rate
fraction_T_{N}: the fraction of CAR-T cell dose in the naïve T cell compartment

The resulting model fits to the CR/PR/NR populations from Fraietta et al. for the five structural variants as compared to the full model are shown in FIG. 29. Note that all model variants are similar with respect to their ability to describe the tumor dynamics but differ in the ability to capture the pharmacokinetics.

Based on the MSE, the original model and variant 5 (inclusion of the T_{N}compartment) best describe the data.

Variant 2, lacking an exhausted state but containing T_{M} and T_{E} (and the reversable transitions) follows, though this version does not adequately capture the pharmacokinetics of the NR population. Variant 4, including all three T cell populations but lacking the reverse T_{E} to T_{M} transition captures the PK data reasonably well, but fails to capture the peak expansion of the CR and PR populations. Version 1 and 3, lacking a TM compartment, both fail to describe, even qualitatively, any of the population pharmacokinetics.

The Akaike Information Criterion was used to rank the models based on fitting error (MSE) vs. complexity (number of parameters):

$$AIC = n \cdot ln [ M S E ] + \frac{2 \cdot k \cdot n}{n - k - 1}$$

Wherein n=number of measurements, k=free parameters and MSE=mean squared error. The results are summarized in Table 4 below. Note that the AIC was originally developed to rank multivariate linear regression models rather than non-linear ODEs and therefore prioritizes limiting free parameters over goodness-of-fit. The results are thus informative, rather than discriminatory, and need to be balanced with more subjective fitting assessments.

TABLE 4
AIC ranking of original and variant models

| Model variant | Free parameters (k) | MSE | AIC |
|---|---|---|---|
| 0 | 27 | 0.148 | 56.94 |
| 1 | 12 | 1.477 | 51.31 |
| 2 | 23 | 0.194 | 24.82 |
| 3 | 18 | 1.398 | 77.38 |
| 4 | 26 | 0.241 | 66.01 |
| 5 | 32 | 0.109 | 140.4 |

Based on the MSE, variant 5 (inclusion of the T_{N} cell compartment) is the most accurate, outperforming the original model (variant ₀). Examination of the PK curves in FIG. 28 reveals this improvement is due to capturing the last time point (12 month) of the NR profile, which increases from the previous (6 month). This may be an artefact of the data (population average) rather than a real phenomenon, implying the model is overfitting. Note that model variant 5 contains five additional parameters as compared to the original model, and the resulting AIC more than doubles, implying this additional complexity adds little value.

Based on relative AIC, model variants 1 and 2 both outperform the original (full) model. Despite poorer fits (MSE), the reduction in free parameters outweigh this in the AIC calculation. However, based on an assessment of the curves, variants 1 and 2 clearly perform worse than the original, as they are incapable of capturing the NR PK profile (variant 2), or any of the PK profiles (variant 1). Thus, consideration of the fitting error, model complexity, and subjective assessment of the PK curves, some may say that the original model outperforms the structural variants.

In some cases, MSE considers the data points to be of equivalent value, while subjectively some datapoints and readouts may be more-or-less significant. Given the tumor dynamic profiles look quite similar across variants, tumor fits may not always be important model selection criterion. In some instances, CAR-T exposure metrics (Cmax and AUC) can be used to predict clinical efficacy. For example and as shown below, the model variants can be evaluated by MSE of the Cmax (log 10-cells) and AUC (log10-cells.day) in addition to the MSE of the data and the sample-size corrected AIC (Table 5, bold indicates top ranked model by metric).

TABLE 5
MSE ranking of original and variant models

| Variant | Parameters (k) | MSE (all data) | AICc (all data) | MSE (CART Cmax) | MSE (CART AUC) |
|---|---|---|---|---|---|
| 0 | 27 | 0.148 | 56.94 | 0.023914 | 701.89 |
| 1 | 12 | 1.477 | 51.31 | 0.238173 | 2633451.7 |
| 2 | 23 | 0.194 | 24.82 | 0.12383 | 1777.4 |
| 3 | 18 | 1.398 | 77.38 | 0.283941 | 1969201.3 |
| 4 | 26 | 0.241 | 66.01 | 0.458429 | 2322.9 |
| 5 | 32 | 0.052 | 140.4 | 0.059707 | 850.18 |

Based on MSE of the data, variant 5 (inclusion of the T_{N} cell compartment) is the most accurate, outperforming the original model (variant 0). Examination of the PK curves in FIG. 29 reveals this improvement is due to capturing the last time point (12 month) of the NR profile, which increases from the previous (6 month). This may be an artefact of the data (population average) rather than a real phenomenon, implying the model is overfitting. Note that model variant 5 contains five additional parameters as compared to the original model, and the resulting AIC more than doubles from 57 to 140, indicating this additional complexity adds little value. To assess generalizability of the model, two pre-clinical datasets with PK and tumor dynamic dose-response data were fit to: CD19-CAR-T treated of NALM xenografts (FIG. 30), and BCMA-CAR-T treated MM1.S xenografts (FIG. 31). In both cases the model described the data with good accuracy.

FIG. 16A shows a flowchart of a method 1600 for training a machine learning classifier (e.g., machine learning classifier 2304 at FIG. 23), according to an embodiment. In some implementations, method 1600 can be performed by a processor (e.g., processor 2301). For example, instructions to cause the processor 2301 to execute the method 1600 can be stored in memory 2302 of FIG. 23. In some implementations, by performing steps such as filtering gene signatures and/or grouping filtered gene signatures, a feature vector can be defined quicker and/or with reduced computational burden compared to known methods. In turn, a machine learning classifier can be trained using the feature vector quicker and/or with reduced computational burden.

At 1601, gene expression data for a plurality of cells are received (e.g., from memory 2302 of compute device 2300; from a compute device communicably coupled to compute device 2300 via a network; etc.). In some implementations, the cells are immune cells. In some implementations, the cells are immune cells, and the immune cells are T cells expressing a chimeric antigen receptor (CAR-T cells). In some implementations, the cells are immune cells. and the immune cells are T cells expressing a chimeric antigen receptor (CAR-T cells), and the immune cells include at least one of autologous cells or allogeneic cells.

At 1602, differential gene expression analysis is conducted based on the gene expression data to generate differential gene expression data (e.g., differentially expressed genes). At 1603, per-sample gene signature enrichment is estimated for a plurality of identified biological pathways based on the differential gene expression data. At 1604, gene signatures having a differential enrichment pattern are filtered based on a pre-determined threshold between groups of responders and non-responders to a treatment. In some implementations, filtering at 1604 is based on statistically significant differences between the groups of responders and non-responders. At 1605, filtered gene signatures are grouped into a plurality of groups based on pairwise correlations between gene signature enrichment scores. At 1606, randomly selecting a gene signature from each group from the plurality of groups to define a set of gene signatures is iteratively performed a predefined number of times to define a plurality of sets of gene signatures. At 1607, the plurality of sets of gene signatures are provided as an input to a feature selection algorithm to define a feature vector including a reduced set of gene signatures identified from the plurality of sets of gene signatures. In some implementations. the feature selection algorithm is a genetic algorithm. At 1608, a machine learning classifier (e.g., machine learning classifier 2304 of FIG. 23) is trained using the feature vector. In some implementations, the machine learning classifier is at least one of a logistic regression-based classifier, a multinomial logistic regression, a decision tree, a perceptron, support vector machines. a K-nearest neighbor, a Naïve Bayes classifier, or a random forest.

FIG. 16B shows a flowchart of a method 1650 for training a machine learning classifier (e.g., machine learning classifier 2304 at FIG. 23), according to an embodiment. In some implementations, method 1650 can be performed by a processor (e.g., processor 2301). For example, instructions to cause the processor 2301 to execute the method 1650 can be stored in memory 2302 of FIG. 23. In some implementations, by performing steps such selecting the those identified biological pathways that are statistically significant, a feature vector can be defined quicker and/or with reduced computational burden compared to known methods. In turn, a machine learning classifier can be trained using the feature vector quicker and/or with reduced computational burden.

At 1651, gene expression data for a plurality of cells are received (e.g., from memory 2302 of compute device 2300; from a compute device communicably coupled to compute device 2300 via a network; etc.). In some implementations, the cells are immune cells. In some implementations, the cells are immune cells, and the immune cells are T cells expressing a chimeric antigen receptor (CAR-T cells). In some implementations, the cells are immune cells, and the immune cells are T cells expressing a chimeric antigen receptor (CAR-T cells), and the immune cells include at least one of autologous cells or allogeneic cells.

At 1652, differential gene expression analysis is conducted based on the gene expression data to generate differential gene expression data (e.g., differentially expressed genes). At 1653, per-sample gene signature enrichment is estimated for a plurality of identified biological pathways based on the differential gene expression data. At 1654, a set of identified biological pathways from the plurality of identified biological pathways that are statistically significant (e.g., top 30 most statistically significant) are defined based on the per-sample gene signature enrichment. At 1655, the set of identified biological pathways are provided as an input to a feature selection algorithm to define a feature vector. At 1608, a machine learning classifier (e.g., machine learning classifier 2304 of FIG. 23) is trained using the feature vector. In some implementations.,the machine learning classifier is at least one of a logistic regression-based classifier, a multinomial logistic regression, a decision tree, a perceptron. support vector machines, a K-nearest neighbor, a Naïve Bayes classifier, or a random forest.

FIG. 17 shows a flowchart of a method 1700 for predicting a clinical outcome of a patient in response to a cell therapy treatment, according to an embodiment. In some implementations, method 1700 can include using a machine learning classifier trained using method 1600. In some implementations, method 1700 can be performed by a processor (e.g., processor 2301). For example, instructions to cause the processor 2301 to execute the method 1700 can be stored in memory 2302 of FIG. 23. In some implementations, a predicted clinical outcome can be generated quicker and/or with reduced computational burden compared to known methods using method 1700. Furthermore, the predicted clinical outcome can be used to determine remedial actions (e.g., administering a treatment, refraining from administering a treatment, etc.) that can be performed by (or prevented from being performed by), for example, a compute device and/or a human.

At 1701, gene expression data for a plurality of cells to be administered to the patient is received. In some implementations, the cells are immune cells. In some implementations, the cells are immune cells, and the immune cells are T cells expressing a chimeric antigen receptor (CAR-T cells). In some implementations, the cells are immune cells, and the immune cells are T cells expressing a chimeric antigen receptor (CAR-T cells), and the immune cells are selected from a group consisting of autologous cells or allogeneic cells. At 1702, per-sample gene signature enrichment for a plurality of identified biological pathways is estimated. At 1703, gene signatures which are differentially enriched are filtered between responder vs non-responder to the cell therapy. In some implementations, filtering at 1703 is based on statistically significant differences between the groups of responders and non-responders. At 1704, filtered gene signatures are provided as an input to a machine learning classifier. In some implementations, the machine learning classifier is at least one of a logistic regression-based classifier, a multinomial logistic regression, a decision tree. a perceptron. support vector machines, a K-nearest neighbor, a Naïve Bayes classifier, or a random forest. In some implementations, the machine learning classifier is trained according to method 1600. At 1705, a predicted clinical outcome is generated. In some implementations, the predicted clinical outcome includes at least one of (i) a complete response, non-response or partial response, (ii), tumor size or burden reduction by a predetermined threshold, or (iii) cytokine release syndrome (CRS) toxicity.

In some implementations, method 1700 further includes a step of comparing predicted clinical outcomes for different cell populations, cell features and/or parameters for cell growth and/or manufacture and identifying those associated with a more favorable clinical outcome.

FIG. 18 shows a flowchart of a method 1800 for generating a predicted clinical outcome, according to an embodiment. In some implementations, method 1800 can be performed by a processor (e.g., processor 2301). For example, instructions to cause the processor 2301 to execute the method 1800 can be stored in memory 2302 of FIG. 23. In some implementations, a predicted clinical outcome can be generated quicker and/or with reduced computational burden compared to known methods using method 1800. Furthermore, the predicted clinical outcome can be used to determine remedial actions (e.g., administering a treatment, refraining from administering a treatment, etc.) that can be performed by (or prevented from being performed by), for example, a compute device and/or a human.

At 1801, a cell population is produced. In some implementations, the cells are immune cells. In some implementations, the cells are immune cells, and the immune cells are T cells expressing a chimeric antigen receptor (CAR-T cells). In some implementations, the cells are immune cells, and the immune cells are T cells expressing a chimeric antigen receptor (CAR-T cells), and the immune cells are selected from a group consisting of autologous cells or allogeneic cells. At 1802, gene expression data for the cell population is received. At 1803, the gene expression data is analyzed to identify a set of gene signatures. At 1804, the set of gene signatures is provided as input to a machine learning classifier (e.g., machine learning classifier 2304 of FIG. 23). At 1805, a predicted clinical outcome is generated (e.g., by the machine learning classifier). In some implementations, the machine learning classifier is at least one of a logistic regression-based classifier, a multinomial logistic regression. a decision tree, a perceptron, support vector machines, a K-nearest neighbor, a Naïve Bayes classifier, or a random forest. In some implementations, the machine learning classifier trained according to method 1600. In some implementations, the predicted clinical outcome includes at least one of (i) a complete response, non-response or partial response, (ii) tumor size or burden reduction by a predetermined threshold, or (iii) cytokine release syndrome (CRS) toxicity.

In some implementations, method 1800 further includes a step of comparing predicted clinical outcomes for different cell populations, cell features and/or parameters for cell growth and/or manufacture and identifying those associated with a more favorable clinical outcome.

In some implementations, method 1800 further includes generating a reduced set of gene signatures from the identified set of gene signatures. In some implementations, analyzing the gene expression data includes estimating per-sample gene signature enrichment for a plurality of identified biological pathways based on the differential gene expression data. In some implementations, analyzing the gene expression data includes filtering gene signatures having differential enrichment pattern based on a pre-determined threshold between groups of responders and non-responders to a treatment. In some implementations, analyzing the gene expression data includes grouping filtered gene signatures into a plurality of groups based on pairwise correlations between gene signature enrichment scores. In some implementations. analyzing the gene expression data includes randomly selecting a gene signature from each group from the plurality of groups to define a set of gene signatures from the plurality of sets of gene signatures iteratively performing a predefined number of times to define a plurality of sets of gene signatures. In some implementations, analyzing the gene expression data includes providing the plurality of sets of gene signatures as an input to a feature selection algorithm to define a feature vector including a reduced set of gene signatures identified from the plurality of sets of gene signatures.

FIG. 19 shows a flowchart of a method 1900 for predicting a clinical outcome of a patient in response to a cell therapy, according to an embodiment. In some implementations. method 1900 can be performed by a processor (e.g., processor 2301). For example. instructions to cause the processor 2301 to execute the method 1900 can be stored in memory 2302 of FIG. 23. In some implementations, the predicted clinical outcome can be used to determine if and/or how a cell therapy should be administered. Such insight can be useful for, for example, refraining from administering a cell therapy to a patient that would likely not lead to a desirable outcome, or causing a cell therapy to be administered to a patient that would likely lead to a desirable outcome. Furthermore, the use of a mechanism-based dynamical model can enable predictions (e.g., pharmacokinetic response, pharmacodynamic response, clinical outcome, etc.) to be arrived at much faster than a human (e.g., given the sheer amount of data to be considered).

At 1901, a tumor burden of a patient and one or more characteristics of a cell therapy are measured. In some implementations, the one or more characteristics of the cell therapy include an initial cell dose and/or cell sub-population characteristics. In some implementations, the cell therapy includes an immune cell therapy. In some implementations. the cell therapy includes an immune cell therapy, the immune cell therapy including a CAR-T cell therapy. In some implementations, the cell therapy includes an immune cell therap, the immune cell therapy including a CAR-T cell therapy, and the immune cell therapy including at least one of an autologous immune cell therapy or an allogeneic immune cell therapy. In some implementations, the tumor burden is determined by at least one of complete blood counts, flow cytometry, radiographic (PET/CT) scans, measurement of circulating tumor-DNA, or indication-specific circulating tumor biomarkers. At 1902, the measured tumor burden and the one or more characteristics of the cell therapy are provided as inputs to a mechanism-based dynamical model (e.g., dynamical model 2305 of FIG. 23). At 1903, using the mechanism-based dynamical model, a pharmacokinetic and/or pharmacodynamic response of the cell therapy in the patient is predicted. In some implementations, the mechanism-based dynamical model predicts a pharmacokinetic and/or pharmacodynamic response of the cell therapy in the patient. At 1904, the clinical outcome of the patient in response to the cell therapy is predicted.

FIG. 20 shows a flowchart of a method 2000 for administering a cell therapy to a patient. according to an embodiment. In some implementations, method 2000 can be performed by a processor (e.g., processor 2301). For example, instructions to cause the processor 2301 to execute the method 2000 can be stored in memory 2302 of FIG. 23. In some implementations, the predicted clinical outcome can be used to determine if and/or how a cell therapy should be administered. Such insight can be useful for, for example, refraining from administering a cell therapy to a patient that would likely not lead to a desirable outcome, causing a cell therapy to be administered to a patient that would likely lead to a desirable outcome, and/or determining an appropriate dosage amount to be administered for a given patient. Furthermore, the use of a mechanism-based dynamical model can enable predictions (e.g., clinical outcome, dosage amount, etc.) to be arrived at much faster than a human (e.g., given the sheer amount of data to be considered).

At 2001, a tumor burden of a patient and one or more characteristics of the cell therapy are measured. In some implementations, the one or more characteristics of the cell therapy include an initial cell dose and/or cell sub-population characteristics. In some implementations. the cell therapy includes an immune cell therapy. In some implementations, the cell therapy includes an immune cell therapy, the immune cell therapy including a CAR-T cell therapy. In some implementations, the cell therapy includes an immune cell therapy, the immune cell therapy including a CAR-T cell therapy, and the immune cell therapy including at least one of an autologous immune cell therapy or an allogeneic immune cell therapy. In some implementations, the tumor burden is determined by at least one of complete blood counts, flow cytometry, radiographic (PET/CT) scans, measurement of circulating tumor DNA, or indication-specific circulating tumor biomarkers.

At 2002. the measured tumor burden and the one or more characteristics of the cell therapy are provided as inputs to a mechanism-based dynamical model (e.g., dynamical model 2305 of FIG. 23). In some implementations, the one or more characteristics of the cell therapy include an initial cell dose and/or cell sub-population characteristics. In some implementations, the mechanism-based dynamical model predicts a pharmacokinetic and/or pharmacodynamic response of the cell therapy in the patient. At 2003, using the mechanism-based dynamical model, the clinical outcome of the patient is predicted. At 2004, the cell therapy is administered to the patient, wherein the dosage administered to the patient is determined by the mechanism-based dynamical model. In some implementations, the cell therapy is caused to be administered to the patient by the processor sending a signal indicating that the cell therapy is to be administered to the patient (e.g., a display, a speaker, a different compute device, etc.). In some implementations. in turn. the cell therapy can be administered to the patient (e.g., by a medial professional, by a robot, etc.). In some implementations, the dosage administered to the patient is determined by the mechanism-based dynamical model based on at least the tumor burden of the patient and the Cmax of the cell therapy.

FIG. 21 shows a flowchart of a method 2100 for producing a cell therapy product. according to an embodiment. In some implementations, method 2100 can be performed by a processor (e.g., processor 2301). For example, instructions to cause the processor 2301 to execute the method 2000 can be stored in memory 2302 of FIG. 23. In some implementations, by performing method 2100, the patient-specific cell dosage can be used to prepare a cell therapy product at the patient-specific cell dosage. In turn, the patient can be administered with the cell therapy product with the cell dosage specified for that patient (e.g., rather than not getting enough, or rather than getting too much).

At 2101, a population of cells having at least one sub-population is provided. In some implementations, the one or more characteristics of the cell therapy include an initial cell dose and/or cell sub-population characteristics. In some implementations, the cell therapy includes an immune cell therapy. In some implementations, the cell therapy includes an immune cell therapy, the immune cell therapy comprising a CAR-T cell therapy. In some implementations, the cell therapy includes an immune cell therapy, the immune cell therapy including a CAR-T cell therapy, and the immune cell therapy including at least one of an autologous immune cell therapy or an allogeneic immune cell therapy.

At 2102, a tumor burden of a patient is measured. In some implementations, the tumor burden is determined by at least one of complete blood counts, flow cytometry, radiographic (PET/CT) scans, measurement of circulating tumor-DNA, or indication-specific circulating tumor biomarkers. At 2103, using a mechanism-based dynamical model (e.g., dynamical model 2305 of FIG. 23), a predicted expansion capacity (Cmax) of the cell therapy product is determined. At 2104, using the mechanism-based dynamical model, a patient-specific cell dosage of the cell therapy product is determined. In some implementations, the patient-specific dosage is determined based on tumor burden and predicted Cmax, such that Cmax/tumor burden ratios are improved (e.g., optimized). In some implementations, the Cmax and patient-specific cell dosage are dependent upon the sub-population of the cell population and the tumor burden of the patient. In some implementations, a sub-population of cells enriched for memory T cells having high proliferation rates, high cytotoxic potency and/or lack of exhaustion are associated with a more favorable clinical outcome.

In some implementations, method 2100 further includes causing preparation of a single dose form containing the determined patient-specific cell dosage. For example, the processor can send a signal to cause the single dose form to be prepared (e.g., by a compute device, by a medical professional, etc.). In some implementations, a patient-specific cell dosage form is produced according to method 2100.

FIG. 22 shows a flowchart of a method 2200 for determining a patient-specific dosage of cell therapy to be administered, according to an embodiment. In some implementations, method 2200 can be performed by a processor (e.g., processor 2301). For example. instructions to cause the processor 2301 to execute the method 2000 can be stored in memory 2302 of FIG. 23. In some implementations, a patient-specific cell dosage form including cells at a dosage determined according to method 2200 is produced. In some implementations, by performing method 2200, the patient-specific cell dosage can be used to prepare a cell therapy product at the patient-specific cell dosage. In turn, the patient can be administered with the cell therapy product with the cell dosage specified for that patient (e.g., rather than not getting enough, or rather than getting too much).

At 2201, a cell population is provided. In some implementations, the one or more characteristics of the cell therapy include an initial cell dose and/or cell sub-population characteristics. In some implementations, the cell therapy includes an immune cell therapy. In some implementations, the cell therapy includes an immune cell therapy, the immune cell therapy including a CAR-T cell therapy. In some implementations, the cell therapy includes an immune cell therapy, the immune cell therapy including a CAR-T cell therapy, and the immune cell therapy including at least one of an autologous immune cell therapy or an allogeneic immune cell therapy.

At 2202, gene expression data for the cell population is received. At 2203, the gene expression data is analyzed to identify a set of gene signatures. At 2204, the set of gene signatures are provided as an input to a machine learning classifier (e.g., machine learning classifier 2304 of FIG. 23) to generate a predicted expansion capacity (Cmax) of the cell population. In some implementations, the machine learning classifier is trained according to method 1600. At 2205, the Cmax of the cell population and a tumor burden of a patient is provided as input to a mechanism-based dynamical model (e.g., dynamical model 2305 of FIG. 23). In some implementations, the tumor burden is determined by at least one of complete blood counts, flow cytometry, radiographic (PET/CT) scans, measurement of circulating tumor-DNA, or indication-specific circulating tumor biomarkers. At 2206, using the mechanism-based dynamical model, a patient-specific cell dosage of the cell therapy product is determined. In some implementations, determining a patient-specific dosage is determined based on tumor burden and predicted Cmax, such that Cmax/tumor burden ratios are improved (e.g., optimized).

FIG. 23 is a schematic block diagram of a compute device 2300, according to an embodiment. The compute device 2300 can be or include a hardware-based computing device and/or a multimedia device, such as, for example, a computer, a desktop. a laptop, a smartphone, and/or the like. The compute device 2300 includes a memory 2302, a communication interface 2303, and a processor 2301.

The memory 2302 of the compute device 2300 can be, for example, a memory buffer. a random-access memory (RAM), a read-only memory (ROM), a hard drive, a flash drive, and/or the like. The memory 2302 can store, for example, code (e.g., programs written in C, C++, Python. etc.) that includes instructions to cause the processor 2301 to perform one or more processes methods, and or functions (e.g., method 1600, 1700, 1800, 1900, 2000, 2100, and/or 2200).

The memory 2302 can include a machine learning classifier 2304 and/or a dynamical model 2305. The machine learning classifier 2304 and/or dynamical model 2305 can be, for example, a machine learning model, an artificial intelligence model, an analytical model, and/or a mathematical model. In some implementations, the machine learning classifier 2304 can be trained (e.g., at compute device 2300 and/or a different compute device) using method 1600. In some implementations, the machine learning classifier 2304 can perform one or more steps discussed in methods 1600, 1700, 1800, and/or 2200. In some implementations, the dynamical model 2305 can perform one or more steps discussed in methods 1900, 2000, 2100, and/or 2200. In some implementations, the machine learning classifier 2304 and/or dynamical model 2305 are stored in a different memory (i.e., not memory 2302) included in a different compute device (i.e., not compute device 2300) communicable coupled to compute device 2300 via a network (not shown in FIG. 23).

The communication interface 2303 of the compute device 2300 can be a hardware component of the compute device 2300 to facilitate data communication between the compute device 2300 and external devices (e.g., a network, a compute device, and/or a server, not shown). The communication interface 2303 can be operatively coupled to and used by the processor 2301 and/or the memory 2302. The communication interface 2303 can be, for example, a network interface card (NIC), a Wi-FiR module, a Bluetooth® module, an optical communication module, and/or any other suitable wired and/or wireless communication interface.

The processor 2301 can be, for example, a hardware based integrated circuit (IC) or any other suitable processing device configured to run or execute a set of instructions or a set of codes. For example, the processor 2301 can include a general-purpose processor, a central processing unit (CPU), an application specific integrated circuit (ASIC), a graphics processing unit (GPU), and/or the like. The processor 2301 is operatively coupled to the memory 2302 through a system bus (for example, address bus, data bus, and/or control bus; not shown). The processor 2301 can be configured to perform method 1600, 1700, 1800, 1900, 2000, 2100, and/or 2200.

In order to address various issues and advance the art, the entirety of this application (including the Cover Page, Title, Headings, Background, Summary, Brief Description of the Drawings, Detailed Description, Claims, Abstract, Figures, Appendices, and otherwise) shows, by way of illustration, various embodiments in which the claimed innovations can be practiced. The advantages and features of the application are of a representative sample of embodiments only and are not exhaustive and/or exclusive. They are presented to assist in understanding and teach the claimed principles.

Examples of computer code include, but are not limited to, micro-code or micro-instructions, machine instructions, such as produced by a compiler, code used to produce a web service, and files containing higher-level instructions that are executed by a computer using an interpreter. For example, embodiments can be implemented using Python, Java, JavaScript, C++, and/or other programming languages, packages, and software development tools.

The drawings primarily are for illustrative purposes and are not intended to limit the scope of the subject matter described herein. The drawings are not necessarily to scale; in some instances, various aspects of the subject matter disclosed herein can be shown exaggerated or enlarged in the drawings to facilitate an understanding of different features. In the drawings, like reference characters generally refer to like features (e.g., functionally similar and/or structurally similar elements).

The acts performed as part of a disclosed method(s) can be ordered in any suitable way. Accordingly, embodiments can be constructed in which processes or steps are executed in an order different than illustrated, which can include performing some steps or processes simultaneously, even though shown as sequential acts in illustrative embodiments. Put differently, it is to be understood that such features may not necessarily be limited to a particular order of execution, but rather, any number of threads, processes, services, servers, and/or the like that may execute serially, asynchronously, concurrently, in parallel, simultaneously, synchronously, and/or the like in a manner consistent with the disclosure. As such, some of these features may be mutually contradictory, in that they cannot be simultaneously present in a single embodiment. Similarly, some features are applicable to one aspect of the innovations, and inapplicable to others.

The phrase “and/or.” as used herein in the specification and in the embodiments, should be understood to mean “either or both” of the elements so conjoined. i.e., elements that are conjunctively present in some cases and disjunctively present in other cases. Multiple elements listed with “and/or” should be construed in the same fashion, i.e., “one or more” of the elements so conjoined. Other elements can optionally be present other than the elements specifically identified by the “and/or” clause, whether related or unrelated to those elements specifically identified. Thus, as a non-limiting example, a reference to “A and/or B”, when used in conjunction with open-ended language such as “comprising” can refer. in one embodiment, to A only (optionally including elements other than B); in another embodiment, to B only (optionally including elements other than A); in yet another embodimen, to both A and B (optionally including other elements); etc.

As used herein in the specification and in the embodiments, “or” should be understood to have the same meaning as “and/or” as defined above. For example, when separating items in a list, “or” or “and/or” shall be interpreted as being inclusive, i.e., the inclusion of at least one, but also including more than one, of a number or list of element, and, optionally, additional unlisted items. Only terms clearly indicated to the contrary, such as “only one of” or “exactly one of,” or, when used in the embodiments, “consisting of,” will refer to the inclusion of exactly one element of a number or list of elements. In general, the term “or” as used herein shall only be interpreted as indicating exclusive alternatives (i.e., “one or the other but not both”) when preceded by terms of exclusivity, such as “either,” “one of,” “only one of,” or “exactly one of.” “Consisting essentially of,” when used in the embodiments, shall have its ordinary meaning as used in the field of patent law.

Some embodiments and/or methods described herein can be performed by software (executed on hardware), hardware, or a combination thereof. Hardware modules may include, for example, a processor, a field programmable gate array (FPGA), and/or an application specific integrated circuit (ASIC). Software modules (executed on hardware) can include instructions stored in a memory that is operably coupled to a processor, and can be expressed in a variety of software languages (e.g., computer code), including C, C++, Java™, Ruby, Visual Basic™, and/or other object-oriented, procedural, or other programming language and development tools. Examples of computer code include, but are not limited to, micro-code or micro-instructions, machine instructions, such as produced by a compiler, code used to produce a web service, and files containing higher-level instructions that are executed by a computer using an interpreter. For example, embodiments may be implemented using imperative programming languages (e.g., C, Fortran, etc.), functional programming languages (Haskell, Erlang, etc.), logical programming languages (e.g., Prolog), object-oriented programming languages (e.g., Java, C++, etc.) or other suitable programming languages and/or development tools. Additional examples of computer code include, but are not limited to, control signals, encrypted code, and compressed code.

We claim:

1. An apparatus, comprising:
a memory; and a hardware processor operatively coupled to the memory, the hardware processor configured to:
receive gene expression data for a plurality of cells;
conduct differential gene expression analysis based on the gene expression data to generate differential gene expression data;
estimate per-sample gene signature enrichment for a plurality of identified biological pathways based on the differential gene expression data;
filter gene signatures having a differential enrichment pattern based on a pre-determined threshold between groups of responders and non-responders to a treatment;
group filtered gene signatures into a plurality of groups based on pairwise correlations between gene signature enrichment scores;
iteratively perform a predefined number of times to define a plurality of sets of gene signatures, randomly selecting a gene signature from each group from the plurality of groups to define a set of gene signatures from the plurality of sets of gene signatures;
provide the plurality of sets of gene signatures as an input to a feature selection algorithm to define a feature vector including a reduced set of gene signatures identified from the plurality of sets of gene signatures; and
train a machine learning classifier using the feature vector.

2. The apparatus of claim 1, wherein the machine learning classifier is at least one of a logistic regression, a multinomial logistic regression, a decision tree, a perceptron, a support vector machine, a K-nearest neighbor, Naïve Bayes classifier, or a random forest.

3. The apparatus of claim 1, where the feature selection algorithm is a genetic algorithm.

4. The apparatus of claim 1, wherein the step of filtering gene signatures is based on statistically significant differences between the groups of responders and non-responders.

5. The apparatus of claim 1, wherein the plurality of cells are immune cells.

6. The apparatus of claim 5, wherein the immune cells are T cells expressing a chimeric antigen receptor (CAR-T cells).

7. The apparatus of claim 6, wherein the immune cells include at least one of autologous cells or allogeneic cells.

8. A method for predicting a clinical outcome of a patient in response to a cell therapy treatment, comprising:
receiving gene expression data for a plurality of cells to be administered to the patient;
estimating per-sample gene signature enrichment for a plurality of identified biological pathways;
filtering gene signatures which are differentially enriched between responder vs non-responder to the cell therapy;
providing filtered gene signatures as an input to a machine learning classifier; and
generating a predicted clinical outcome.

9. The method of claim 8, wherein the machine learning classifier is a logistic regression-based classifier.

10. The method of claim 8, wherein the step of filtering gene signatures is based on statistically significant differences between the groups of responders and non-responders.

11. The method of claim 8, wherein the cells are immune cells.

12. The method of claim 11, wherein the immune cells are T cells expressing a chimeric antigen receptor (CAR-T cells).

13. The method of claim 12, wherein the immune cells include at least one of autologous cells or allogeneic cells.

14. The method of claim 8, wherein the machine learning classifier is trained using a feature vector that includes a reduced set of gene signatures identified from a plurality of sets of gene signatures, the plurality of sets of gene signatures defined by repeatedly randomly selecting a gene signature from each group from a plurality of groups to define a set of gene signatures from the plurality of sets of gene signatures, filtered gene signatures grouped into the plurality of groups based on pairwise correlation between gene signature enrichment scores.

15. The method of claim 8, wherein the method further comprises a step of comparing predicted clinical outcomes for different cell populations, cell features or parameters for cell growth or manufacture and identifying those associated with a more favorable clinical outcome.

16. The method of claim 8, wherein the predicted clinical outcome includes at least one of (i) a complete response, non-response or partial response, (ii) tumor size or burden reduction by a predetermined threshold, or (iii) cytokine release syndrome (CRS) or other toxicity.

17. The method of claim 8, wherein the providing includes providing the filtered gene signatures as the input to the machine learning classifier to generate a predicted expansion capacity (Cmax) of the plurality of cells, the method further comprising:
providing, as input to a mechanism-based dynamical model, the Cmax of the plurality of cells and a tumor burden of the patient;
determining, using the mechanism-based dynamical model, a patient-specific cell dosage of the plurality of cells; and
administering, in response to generating the predicted clinical outcome, the patient-specific cell dosage of the plurality of cells to the patient.

18. A method, comprising:
receiving gene expression data for a plurality of cells;
conducing differential gene expression analysis based on the gene expression data to generate differential gene expression data;
estimating per-sample gene signature enrichment for a plurality of identified biological pathways based on the differential gene expression data;
defining, based on the per-sample gene signature enrichment, a set of identified biological pathways from the plurality of identified biological pathways that are statistically significant;
providing the set of identified biological pathways as an input to a feature selection algorithm to define a feature vector; and
training a machine learning classifier using the feature vector.

19. The method of claim 18, wherein the plurality of cells are T cells expressing a chimeric antigen receptor (CAR-T cells).

20. The method of claim 18, wherein the plurality of cells includes at least one of autologous cells or allogeneic cells.
